(19) United States
(12) Patent Application Publication
(10) Pub. No.: US 2024/0181034 A1
(43) Pub. Date: Jun. 6, 2024

(54) HUMAN METAPNEUMO VIRUS VACCINE

(71) Applicant: Valneva SE, Saint-Herblain (FR)

(72) Inventors: Urban Lundberg, Pressbaum (AU); Andreas Meinke, Pressbaum (AU); Fabien Perugi, Nantes (FR)

(73) Assignee: Valneva SE, Saint-Herblain (FR)

(21) Appl. No.: 18/285,416

(22) PCT Filed: Apr. 8, 2022
(86) PCT No.: PCT/EP2022/059492

§ 371 Date: Oct. 3, 2023

(30) Foreign Application Priority Data
Apr. 9, 2021 (EP) 21167609.3

(51) Int. Cl. A61K 39/12; A61K 39/00; A61P 31/14; C12N 7/00

(52) U.S. Cl. CPC A61K 39/12; A61P 31/14; C12N 7/00; A61K 2039/55505; A61K 2039/55561; A61K 2039/55566; A61K 2039/55572; A61K 2039/575; C12N 2760/18322; C12N 2760/18334

(57) ABSTRACT

The present invention relates to a vaccine composition for preventing and/or treating a respiratory system infection such as a human metapneumovirus infection of the respiratory system. This vaccine composition comprises one, two or more modified human metapneumovirus (hMPV) F proteins or variants thereof provided in a pre-fusion -fusion conformation form.

### FIELD OF THE INVENTION

The present invention relates to a vaccine composition for preventing and/or treating a respiratory system infection such as a human metapneumovirus infection of the respiratory system. This vaccine composition comprises i) one or II) two or more modified human metapneumovirus (hMPV) F proteins or variants thereof provided in a pre-fusion conformation form.

### BACKGROUND OF THE INVENTION

Currently no vaccine or specific antiviral drug is available to prevent or treat hMPV infections in subjects such as humans or animals. Infants, some categories of young children below 5 years, elderly above 65 years and immunocompromised patients are particularly at risk to develop severe bronchiolitis or pneumonia due to a hMPV infection. However, vaccine development is challenging because for example the neutralizing antibody response induced by a natural hMPV infection is unfortunately not long lasting, declines over time and wherein the specific memory B cell response is weak (Falsey, A.R.: Hennessey, P.A.; Formica, M.A.; Criddle, M.M.: Biear, J.M.: Walsh, E.E. Humoral immunity to human metapneumovirus infection in adults. Vaccine 2009. 28. 1477-1480).

Phylogenic analysis of genomic sequences from various hMPV strains and clinical isolates revealed two main genotypes (lineages), namely A and B, each divided into subgroups (five sub-lineages or subgroups), A1/A2a/A2b, and B1/B2 (van den Hoogen BG. Herfst S. Sprong L. Cane PA. Forleo-Neto E, Swart RL de, Osterhaus ADME, Fouchier RAM. (2004) Antigenic and genetic variability of human metapneumoviruses. Emerging infectious diseases 10(4): 658-66).

Protection against hMPV is mainly afforded by neutralizing antibodies directed against the fusion (F) glycoprotein (Williams et al. (2007) A Recombinant Human Monoclonal Antibody to Human Metapneumovirus Fusion Protein That Neutralizes Virus In Vitro and Is Effective Therapeutically In Vivo. J Virol 81(15): 8315-8324: Battles et al. (2017) Nat Commun. 16:8(1): 1528). The F protein is immunodominant and quite conserved between hMPV strains. Rare mutations in the F protein do not result in precarious loss of neutralizing epitopes, so that hMPV genotypes and subgroups are quite stable genetically over time (Yang CF. Wang CK. Tollefson SJ. Piyaratna R. Lintao LD. Chu M. Liem A, Mark M, Spaete RR, Crowe JE, Jr. Williams JV. (2009) Genetic diversity and evolution of human metapneumovirus fusion protein over twenty years. Virol J 6:138). Cross-protection between genotypes (A and B) and subgroups (A1, A2a, A2b, B1 and B2) was obtained in some animal models, but data are controversial. For instance, induction of cross-protective immunity upon immunization with the soluble F protein isolated from A or B genotype was demonstrated in hamsters (Nerfst et al. 2007. Journal of General Virology (2007), 88, 2702 2709). Conversely, the in vitro study performed with sera from ferrets infected with one genotype did not neutralize the virus of another genotype (Kahn J.S. (2006) Epidemiology of human metapneumovirus. Clin Microbiol 19(3):546-557). The immunogenic response to different hMPV genotypes in humans is not yet well-understood (Rahman et al. (2018) Epidemiological studies in Bangladesh. J Med Virology 2018:1-6). Therefore, circulation of numerous hMPV variants may create complications for developing a vaccine with a broad coverage.

The hMPV F protein mediates fusion of the viral membrane with the cellular membrane to allow viral ribonucleoprotein entry into the cell cytoplasm and initiation of virus replication (Cox RG. Livesay SB, Johnson M, Ohi MD, Williams JV (2012) The human metapneumovirus fusion protein mediates entry via an interaction with RGD-binding integrins. J Virol 86: 12148 12160). The F protein is a type I integral membrane protein that comprises at its C-terminus a hydrophobic transmembrane (TM) domain anchoring the protein in the viral membrane and a short cytoplasmic tail. The native F protein is synthesized as an inactive single-chain precursor F0, which is activated after cleavage by a cell protease generating two polypeptide chains, F1 and F2 (see FIG. 1). The biologically active hMPV F protein exists in two conformations: the metastable pre-fusion and the highly stable post-fusion form (see FIG. 2). Published crystal structures of the pre-fusion and post-fusion forms (revealed essential differences between two conformations that might have effect on immunogenic and antigenic characteristics of the F protein (Melero JA & Más V. (2015) The Pneumovirinae fusion (F) protein: A common target for vaccines and antivirals. Virus Research 209: 128-135).

Several studies showed that both pre-fusion and post-fusion F protein forms possess antigenic epitopes and are able to elicit neutralizing antibodies (Wen et al. (2012) Structure of the Human Metapneumovirus Fusion Protein with Neutralizing Antibody Identifies a Pneumovirus Antigenic Site. Nat Struct Mol Biol. 19(4): 461 463: Battles et al. (2017) Nat Commun. 16,8(1): 1528; Huang et al. (2019) Antibody Epitopes of Pneumovirus Fusion Proteins. Front Immunol. 10, 2778, review). For instance, Melero's group demonstrated that the recombinant pre-fusion F protein induced neutralizing antibodies and immunogenicity studies in animals (Melero JA & Más V. (2015) The Pneumovirinae fusion (F) protein: A common target for vaccines and antivirals. Virus Research 209:128-135; Michael B Battles, Vicente Más, Eduardo Olmedillas, Olga Cano, Mónica Vázquez, Laura Rodriguez, José A Melero, Jason S Mclellan, Nat Commun. 2017 Nov 16:8(1):1528. doi: 10.1038 s41467-017-01708-9). In another study, it was shown that the recombinant post-fusion F protein was able to deplete hMPV-neutralizing antibodies from seropositive human sera (Más V, Rodriguez L, Olmedillas E, Cano O), Palomo C, Terrón MC, Luque D, Melero JA, Mclellan JS. (2016) Engineering. Structure and Immunogenicity of the Human Metapneumovirus F Protein in the Postfusion Conformation. PLOS pathogens. 12(9)). One more group has disclosed modifications in the F protein leading to stabilization of the pre-fusion conformation and their applicability for vaccine development (see U.S. Pat. No. 1,0420,834).

Previously, we have demonstrated induction of high titer neutralizing antibodies and protection of mice upon immunization with the stabilized pre-fusion form of the hMPV F protein (see WO2020234300 A1). In this study, five F protein candidates formulated as single immunogens have shown promising protective efficacy in the lung colonization/infection model, MNA, FFA and/or RT-qPCR model preferably if the right subgroup candidate A1, preferably the L7F_A1_23 (SEQ ID NO: 11) is selected or a combination of A1 and B1 subgroup of one of the five F protein candidates, preferably the L7F_A1_23 (SEQ ID NO: 11) and the L7F_B1_23 (SEQ ID NO: 12). These improved vaccines in a simple format (monovalent or bivalent only) that are more effective against multiple hMPV strains and clinical isolates are important. To date, no attempts to combine the pre-fusion conformations (or test them in a monovalent format) of the F protein in a vaccine formulation have been described in a clinical trial. In another, the post-fusion sF_A1_MFur is also a preferred candidate in the immunogenic composition of the invention.

### SUMMARY OF THE INVENTION

The present invention provides compositions i) comprising one or more modified recombinant hMPV F proteins or variants thereof provided in the pre-fusion conformations: or ii) comprising the combination of one or more modified recombinant hMPV F proteins or variants thereof provided in the pre-fusion conformations. These modified recombinant proteins are derived from the different hMPV genotypes, A and B, or from the same genotype, but different subgroups, or both, preferred are monovalent vaccines or immunogenic compositions thereof (i.e. in particular the L7F_A1_23 (SEQ ID NO: 11)) and bivalent vaccines or immunogenic compositions thereof (i.e. (i.e. in particular the L7F_A1_23 (SEQ ID NO: 11) and L7F_B1_23 (SEQ ID NO: 12). The present invention further provides protein constructs and expression vectors for producing said modified recombinant proteins. The present invention also provides immunogenic compositions (such as vaccines) able to induce specific immune responses and/or enable to provide protection against a hMPV infection and/or in particular also cross-neutralize and protect other subgroups and/or genotypes of hMPV. Furthermore, use of specific combinations of two or more, preferably two, pre-fusion F proteins allows achieving protection against homologous and heterologous hMPV strains. The present invention also relates to methods of producing disclosed recombinant proteins and immunogenic compositions, as well as methods of using them for treating and/or preventing human or animal subjects with mild, moderate or severe hMPV infections.

The problem underlying the present invention is to develop an immunogenic composition (vaccine) that would potentiate strong and long-lasting immune responses and provide better protection against various hMPV strains and clinical isolates than known immunogenic compositions containing, e.g. a single hMPV F protein existing in the pre-fusion conformation which cross-neutralize and/or cross-protect a subgroup and/or other genotype of the hMPV family (A1, A2a, A2b, B1, B2 subgroups, respectively A and B of hMPV) allowing for a simple antigen design and thus very reasonable production costs (simpler production, simpler quality assessment etc.).

The problem underlying this invention is solved by providing compositions comprising only one or two different (different subgroup and/or genotype) F proteins or variants thereof provided in the pre-fusion conformation forms. Moreover, such a solution also includes two or more F proteins formulated in one composition derived from different hMPV strains that belong to the same or distinct genotypes but still providing a more simple design than adding just all of the different subgroups in the vaccine/immunogenic composition.

In order to solve the problem, a couple of F protein candidates from different hMPV genetic groups and subgroups thereof were produced as modified (i.e. stabilized in the pre-fusion conformation) recombinant proteins and studied in several combinations with each other for immunogenicity and protective efficacy in a mouse challenge model or other functional models. In particular, mice immunized with the combination of pre-fusion F proteins from subgroup A1 and B1 or single pre-fusion F proteins were challenged with the virus of subgroup A2a, A2b and/or B1 and induction of neutralizing antibodies and viral load were tested. Alternatively, mice immunized with the combination of pre-fusion F proteins from subgroup A1 and/or B1 or single pre-fusion F proteins can be challenged with the virus of subgroup A1, A2a, A2b and/or B1. Otherwise, protection of mice immunized with the combination of pre-fusion F proteins or single pre-fusion F proteins from subgroup A1 and/or B1 can be evaluated after challenge with the hMPV subgroup A2a, A2b or B2 or other iterations. As the result, cross-protection between two genotypes A and B and different subgroups is observed.

According to one embodiment, a modified (stabilized) F protein of the composition is present in the pre-fusion conformation. Said pre-fusion F protein consists of a single-chain polypeptide similar to the F ectodomain, but lacking the protease cleavage site and the fusion peptide (FP) between F1 and F2 domains. Instead, the single-chain F protein comprises a heterologous peptide linker between F1 and F2 domains, which contains at least one cysteine residue forming a non-natural disulfide (S—S) bond with another cysteine residue in the F1 domain and thus stabilizing the pre-fusion conformation. Alternatively, the pre-fusion hMPV F protein may comprise two polypeptide chains, i.e. F1 and F2 domains covalently linked by two or more S—S bonds. Such protein may contain mutation(s) stabilizing the pre-fusion conformation.

According to yet another embodiment, a further second F protein of the composition is a modified (stabilized) F protein present also in the pre-fusion conformation. Said pre-fusion F protein consists of a single-chain polypeptide similar to the F ectodomain, but lacking the protease cleavage site and the fusion peptide (FP) between F1 and F2 domains. Instead, the single-chain F protein comprises a heterologous peptide linker between F1 and F2 domains, which contains at least one cysteine residue forming a non-natural disulfide (S—S) bond with another cysteine residue in the F1 domain and thus stabilizing the pre-fusion conformation. Alternatively, the pre-fusion hMPV F protein may comprise two polypeptide chains, i.e. F1 and F2 domains covalently linked by two or more S—S bonds. Such protein may contain mutation(s) stabilizing the pre-fusion conformation. According to another embodiment of the invention, the pre-fusion F protein may comprise an amino acid sequence having at least 80%, 85%, 90%, 95%, 96%, 97%, 98% or 99% sequence identity to the amino acid sequence of the parental F protein. A modified F protein having a high sequence identity with a reference parental F protein is also referred to herein as a variant. Generally, homologs or variants of a protein possess a relatively high degree of sequence identity when aligned using standard methods well known in the art (preferred is a global alignment of the to be investigated sequence when comparing to other sequence, e.g. Needleman-Wunsch algorithm using standard settings). Importantly, a homologous F protein or variant is similarly immunogenic and protective as the parental F protein as e.g. measured in the in vitro assay of this document, e.g. the MNA, FFA or PCR used described elsewhere herein.

Additionally, the pre-fusion F proteins of the present invention are recombinant proteins without transmembrane domain (referred herein also as “TM”) and/or cytoplasmic tails produced in heterologous host cells as homo- or preferably as hetero- or homo-trimers. To facilitate the trimerization process, one or more specific modification(s) or trimerization helper domain(s) may be introduced into the C-terminal part of the F protein.

According to yet another embodiment, one pre-fusion F protein are formulated in a single composition further comprising a pharmaceutically exactable carrier and/or excipient. Beside the F proteins, such composition may comprise one or more additional antigen, for instance, another hMPV protein or another antigen directed to another pathogen causing infection of the respiratory system.

Typically, the composition of the present invention is an immunogenic composition (a vaccine) able to elicit hMPV neutralizing antibodies and a specific T cell response directed against hMPV. Optionally, the immunogenic composition may further comprise an adjuvant for enhancing such immune response and/or shifting the immune response to a desirable Th1-type direction. Generally. an immune response (neutralizing antibody titer) induced by the immunogenic composition of the present invention is sufficient to protect against an hMPV infection. Additionally, the immunogenic composition comprising the F protein or variants thereof in both conformation forms elicits an immune response (neutralizing antibody titer) superior to the immune response (neutralizing antibody titers) elicited by an equal amount of the single F protein present either in the pre-fusion conformation.

Furthermore, the immunogenic compositions of the present invention are able to provide protection against more than one hMPV strain, particularly against strains that belong to different genotypes or different subgroups of one genotype. For instance, the immunogenic composition can provide protection against A1 and/or A2a, A2b subgroup(s), alternatively, against B1 and/or B2 subgroup(s), or against both A and B genotypes. Especially, cross-protection between A and B genotypes is desirable.

According to the present invention, the immunogenic compositions (vaccines) of the present invention are useful for the treatment and/or prevention of human and/or animal subjects against a hMPV infection, but other indications such as treatment and/or prevention of mild, severe, hospitalization or death caused by the hMPV infection are also potential target indications of the compositions of the inventions.

In a further embodiment, the present invention provides a method for generating an immune response with a modified F protein or a variant thereof (including combinations) available in the pre-fusion conformation. Such method comprises administering to the subject a therapeutically effective amount of an immunogenic composition containing the pre-fusion forms of the F protein.

In yet one embodiment, the present invention provides a method for treating and/or preventing subjects against hMPV infection or associated disease. Accordingly, the immunogenic composition (vaccine) is administered to a subject via a parenteral route (e.g. intramuscular, intradermal, or subcutaneous) or a mucosal route (e.g. intranasal, oral). As the result, high titers of anti-F protein neutralizing antibodies are generated that assure protection of the immunized subject against hMPV. In a preferred embodiment, the present vaccine induces protective immune responses against more than one hMPV strain, more preferably, against hMPV strains of the same genotype, most preferably, against both genotypes, A and B. In yet one embodiment, the dosage of the vaccine is sufficient to provide a robust anti-hMPV protection against a hMPV infection. Additionally, the method may comprise a prime-boost immunization with the same or different immunogenic compositions comprising modified F proteins or variants thereof derived from the different hMPV subgroups and/or genotypes. For instance, the prime immunization may be done with the vaccine comprising F proteins of genotype A of the invention, while the boost immunization may be done with the vaccine comprising F proteins of genotype B of the invention. In such a way, even better cross-protection between genotypes A and B can be achieved. Furthermore, the method may comprise only a boost immunization with the same or different immunogenic compositions comprising modified F proteins or variants thereof derived from the different hMPV subgroups and/or genotypes in particularly for elderly or adults (e.g. adults at risks) since most of these populations have already been exposed.

Furthermore, the present invention provides a method for producing the recombinant modified F proteins existing in the stabilized pre-fusion conformations and immunogenic compositions comprising these proteins. The aforementioned method includes the following steps: i) expressing the recombinant modified F proteins from the corresponding nucleic acid molecules inserted in expression vectors in host cells, ii) purifying said recombinant F proteins: and iii) combining said purified recombinant proteins with the pharmaceutically acceptable carrier and/or excipient, and optionally with an adjuvant in a pharmaceutical composition or vaccine.

More in particular the following embodiments are provided:
1. An immunogenic composition consisting essentially of a stabilized pre-fusion conformation form of the human metapneumovirus (hMPV) F protein or fragment thereof as the only hMPV antigen and optionally one or more adjuvants and/or at least one pharmaceutically exactable carrier or excipient: wherein said hMPV protein is derived from one subgroup of genotype A or B, and wherein said immunogenic composition cross-neutralizes the hMPV from another subgroup and/or genotype.
2. The composition of embodiment 1, wherein the stabilized pre-fusion conformation form of the human metapneumovirus (hMPV) F protein or fragment thereof is of the A1 subgroup.
3. The composition of embodiment 1-2, wherein the composition consists essentially of i) a stabilized pre-fusion conformation form of the human metapneumovirus (hMPV) F protein or fragment thereof of the A genotype and ii) a stabilized pre-fusion conformation form of the human metapneumovirus (hMPV) F protein or fragment thereof of the B genotype: and optionally one or more adjuvants and/or at least one pharmaceutically exactable carrier or excipient: wherein said immunogenic composition cross-neutralizes the other subgroup and/or other.
4. The composition of embodiment 3, wherein the stabilized pre-fusion conformation form of the human metapneumovirus (hMPV) F protein or fragment thereof of the A genotype is of the A1 subgroup and wherein the stabilized pre-fusion conformation form of the human metapneumovirus (hMPV) F protein or fragment thereof of the B genotype is of the B1 subgroup.
5. The immunogenic composition of any preceding embodiment, wherein the pre-fusion F protein is the recombinant protein.
6. The immunogenic composition of any preceding embodiment, wherein the pre-fusion F protein lacks the cytoplasmic tail and/or transmembrane domain.
7. The immunogenic composition of any preceding embodiment, wherein the pre-fusion F protein has an amino acid sequence, which is a modified amino acid sequence of the native F protein derived from the hMPV strain or clinical isolate.
8. The immunogenic composition of embodiment 9, wherein the native F protein sequence is selected from the group consisting of the amino acid sequences of SEQ ID NO: 1 to 10 that are derived from the hMPV strains NL/1/00, NL/17/00, TN/94-49, NCL174, CAN97-83, NL/1/9, NDL00-1, C1-334, CAN97-82 and TN/89-515.
9. The immunogenic composition of any preceding embodiment, wherein the pre-fusion F protein comprises at least one mutation (substitution or deletion), preferably up to 10 mutations, relative to the native F protein sequence of SEQ ID NO: 1 to 10.
10. The immunogenic composition of any preceding embodiment, wherein the pre-fusion F protein comprises one or more amino acid substitution(s) to cysteine, which introduce one or more non-native disulfide bond(s) that stabilize the pre-fusion conformation.
11. The immunogenic composition of embodiment 10, wherein the cysteine substitution is introduced at
any one of positions 103-120 and any one of positions 335-345;
any one of positions 107-118 and any one of positions 335-342:
any one of positions 117-129 and any one of positions 256-261;
any one of positions 87-102 and any one of positions 117-127;
any one of positions 102-113 and any one of positions 117-127:
any one of positions 102-113 and any one of positions 87-102;
any one of positions 337-341 and any one of positions 421-426;
any one of positions 112-120 and any one of positions 424-432:
any one of positions 150-156 and any one of positions 392-400;
any one of positions 112-120 and any one of positions 370-377:
any one of positions 365-375 and any one of positions 455-465:
any one of positions 365-375 and any one of positions 105-115; or
any one of positions 60-70 and any one of positions 175-185,
wherein the positions correspond to the amino acids of the native F protein sequence of SEQ ID NO: 1 to 10 and 49.
12. The immunogenic composition of any preceding embodiment, wherein the pre-fusion F protein consists of a single polypeptide chain stabilized by at least one non-natural disulfide bond.
13. The immunogenic composition of embodiment 12, wherein the single-chain pre-fusion F protein lacks a protease cleavage site between F1 and F2 domains relative to the native F protein.
14. The immunogenic composition of embodiment 12 and 13, wherein the single-chain pre-fusion F protein comprises a substitution of arginine at position 102 relative to the amino acid positions of the native F protein for another amino acid, preferably glycine.
15. The immunogenic composition of embodiments 12 to 14, wherein the amino acid residues at positions 103-118 of the native F protein are replaced with a heterologous linker consisting of 1 to 5 amino acid residues including cysteine residue, wherein said cysteine residue forms a disulfide bond with a cysteine residue in the F1 domain.
16. The immunogenic composition of embodiment 15, wherein the heterologous linker comprises at least one alanine, glycine or valine residue, preferably the linker has the sequence CGAGA or CGAGV.
17. The immunogenic composition of embodiments 12 to 16, wherein the pre-fusion F protein comprises one or more substitution(s) at positions corresponding to positions 49, 51, 67, 80, 137, 147, 159, 160, 161, 166, 177, 258, 266, 480 and/or 481 of the native hMPV F protein.
18. The immunogenic composition of embodiment 17, wherein the substitution is selected from the group consisting of T49M, E80N, I137W, A147V, A159V, T160F, A161M, I67L, I177L, F258I, S266D, I480C and/or L481C.
19. The immunogenic composition of embodiments 12 to 18, wherein the single-chain pre-fusion F protein comprises one of the following substitution combinations:
N97Q, R102G and G294E;
N97Q, R102G, T160F, I177L and G294E:
N97Q, R102G, T49M, I67L, A161M, E80N, F2581 and G294E;
N97Q, R102G, T49M, I67L, A161M, E51C, K166C, S266D, G294E, I480C and L481C: or
N97Q, R102G, T49M, A161M, I137W, A159V, A147V, I177L and G294E.
20. The immunogenic composition of any of embodiments 12 to 19, wherein the pre-fusion F protein comprises or consists of the amino acid sequence of SEQ ID NO: 11 (L7F_A1_23)
21. The immunogenic composition of any of embodiments 12 to 19, wherein the pre-fusion F protein comprises or consists of the amino acid sequence of SEQ ID NO: 12 (L7F_B1_23).
22. The immunogenic composition of any of embodiments 12 to 19, wherein the pre-fusion F protein comprises or consists of the amino acid sequence of SEQ ID NO: 13 (L7F_A1_23.2).
23. The immunogenic composition of any of embodiments 12 to 19, wherein the pre-fusion F protein comprises or consists of the amino acid sequence of SEQ ID NO: 14 (L7F_B1_23.2).
24. The immunogenic composition of any of embodiments 12 to 19, wherein the pre-fusion F protein comprises or consist of the amino acid sequence of SEQ ID NO: 15 (sF_A1_K_L7).
25. The immunogenic composition of any of embodiments 12 to 19, wherein the pre-fusion F protein comprises or consists of the amino acid sequence of SEQ ID NO: 16 (L7F_A1_31).
26. The immunogenic composition of any of embodiments 12 to 19, wherein the pre-fusion F protein comprises or consists of the amino acid sequence of SEQ ID NO: 17 (L7F_A1_33).
27. The immunogenic composition of any of embodiments 12 to 19, wherein the pre-fusion F protein comprises or consists of the amino acid sequence of SEQ ID NO: 18 (construct L7F_A1_4.2).
28. The immunogenic composition of any of embodiments 1 to 11, wherein the pre-fusion F protein is a two-polypeptide-chain protein and comprises or consists of the amino acid sequence of SEQ ID NO: 19.
29. The immunogenic composition of any of embodiments 1 to 11, wherein the pre-fusion F protein is a two-polypeptide-chain protein and comprises or consists of the amino acid sequence of SEQ ID NO: 20.
30. The immunogenic composition of any of embodiments 1 to 11, wherein the stabilized post-fusion F protein comprises the deletion of the amino acid residues at positions 103 to 111, replacement of R102 by a linker KKRKRR and the substitution G294E relative to the amino acid positions of the native F protein.
31. The immunogenic composition of any of embodiments 1 to 30, wherein the pre-fusion F protein: i) comprises the amino acid sequence having at least 80% sequence identity to the amino acid sequence selected from the group consisting of SEQ ID NO: 1 to 20, and ii) its immunogenicity is similar to immunogenicity of the parental F protein of SEQ ID NO: 1 to 20.
32. The immunogenic composition of any of embodiments 1 to 30, wherein the pre-fusion F protein i) comprises the amino acid sequence having at least 90% sequence identity to the amino acid sequence selected from the group consisting of SEQ ID NO: 1 to 20, and ii) its immunogenicity is equal or similar to immunogenicity of the parental F protein of SEQ ID NO: 1 to 20.
33. The immunogenic composition of any of embodiments 1 to 30, wherein the pre-fusion F protein i) comprises the amino acid sequence having at least 95% sequence identity to the amino acid sequence selected from the group consisting of SEQ ID NO: 1 to 20, and ii) its immunogenicity is equal or similar to immunogenicity of the parental F protein of SEQ ID NO: 1 to 20.
34. The immunogenic composition of any preceding embodiment, wherein the pre- and post-fusion hMPV F protein comprises a trimerization helper domain (foldon) having the sequence of SEQ ID NO: 23 to 28 or a variant thereof.
35. The immunogenic composition of any preceding embodiment, wherein the F protein is produced as a homo- or hetero-trimer.
36. The immunogenic composition of any preceding embodiment, wherein the composition comprises a further non-hMPV antigen.
37. The immunogenic composition of any preceding embodiment, wherein the adjuvant is selected from the group consisting of alum, CpG, such as CpG1018, ODN, I-ODN, IC31®, MF59®, AddaVax™, AS03, AS01, QS21, MPL, GLA-SE, GLA-3M-052-LS, 3M-052-alum or combinations thereof.
38. The immunogenic composition of any preceding embodiment, wherein the adjuvant consists of two or more adjuvants that are selected from the group consisting of alum, CpG, such as CpG1018, ODN, I-ODN, IC31®, MF59®, AddaVax™, AS03, AS01, QS21, MPL, GLA-SE, GLA-3M-052-LS and 3M-052-alum.
39. The immunogenic composition of any preceding embodiment, wherein the adjuvant is alum.
40. The immunogenic composition of any preceding embodiment, wherein the adjuvant is IC31 ®.
41. The immunogenic composition of any preceding embodiment, wherein the adjuvant is GLA-SE.
42. The immunogenic composition of any preceding embodiment, wherein the adjuvant is 3M-052-alum.
43. immunogenic composition of any preceding embodiment, wherein the adjuvant is GLA-3M-052-LS.
44. The immunogenic composition of any preceding embodiment, wherein the adjuvant consists of alum and CpG1018.
45. The immunogenic composition of any preceding embodiment, wherein the adjuvant consists of alum and MPL.
46. The immunogenic composition of any preceding embodiment, wherein the adjuvant consists of alum and IC31®.
47. The immunogenic composition of any preceding embodiment, wherein the adjuvant is AddaVax™.
48. The immunogenic composition of any preceding embodiment, wherein the composition is capable to elicit neutralizing antibodies against the pre-fusion F protein.
49. The immunogenic composition of any preceding embodiment, wherein the composition comprising the pre-fusion protein or the combination of pre-fusion proteins provides a superior immune response (neutralizing antibody titers) as compare to immune response (neutralizing antibody titers) elicited by a composition comprising the post-fusion F protein used at the same total protein amount.
50. The immunogenic composition of any preceding embodiment, wherein the composition provides protection against more than one hMPV strain.
51. The immunogenic composition of any preceding embodiment, wherein the composition provides protection against the hMPV strains of genotype A.
52. The immunogenic composition of any preceding embodiment, wherein the composition provides protection against the hMPV strains of genotype B.
53. The immunogenic composition of any preceding embodiments, wherein the composition provides protection against the hMPV strains of genotype A and genotype B.
54. The immunogenic composition of any preceding embodiment, wherein the composition is a vaccine.
55. The immunogenic composition according to any preceding embodiment for use as a medicament.
56. The immunogenic composition according to any preceding embodiment for treating and/or preventing hMPV infection and associated disease in a subject.
57. A method for generating an immune response to the hMPV F protein in a subject, wherein the method comprises administering to the subject an effective amount of the immunogenic composition according to any previous embodiment 1 to 48.
58. The method of embodiment 56, wherein the immunogenic composition is administered intramuscularly, intradermally, subcutaneously, mucosally, intrarectally, or orally.
59. The method of embodiments 56 and 57, wherein the method comprises a prime-boost administration of the immunogenic composition according to any of embodiments 1 to 55, wherein the prime-boost is done with the same immunogenic composition.
60. The method of embodiments 56 and 58, wherein the method comprises a prime-boost administration of the immunogenic composition according to any of embodiments 1 to 55, wherein the prime administration is done with the composition comprising the F protein of the genotype A and the boost administration is done with the composition comprising the F protein of the genotype B, or vice versa or the method of embodiments 56 and 58, wherein the method comprises a boost administration of the immunogenic composition according to any of embodiments 1 to 55, wherein the prime administration was done with an immunogenic composition comprising a F protein of the genotype A and the boost administration is done with the composition comprising the F protein of the genotype B, or vice versa.
61. A method for treating and/or preventing hMPV infection in a subject, wherein the method comprises administering to the subject a therapeutically effective amount of the immunogenic composition according to any of embodiments 1 to 55 in order to generate neutralizing antibodies against the pre-fusion hMPV F protein and provide protection against the hMPV strains of at least one genotype A or B, preferably both.
62. A method for producing the immunogenic composition according to any of embodiments 1 to 55, wherein the method comprises i) expression of the recombinant pre-fusion F protein from the corresponding nucleic acid molecule inserted in an expression vector in a host cell, ii) purifying the expressed recombinant F protein: and iii) combining the purified recombinant protein with a pharmaceutically acceptable carrier and/or excipient, optionally with an adjuvant.
63. An immunogenic composition consisting essentially of a stabilized post-fusion conformation form of the human metapneumovirus (hMPV) F protein or fragment thereof as the only hMPV antigen; wherein said hMPV protein is derived from one subgroup of genotype A or B, and wherein said immunogenic composition cross-neutralizes the hMPV from another subgroup and/or genotype.
64. The composition of embodiment 63, wherein the stabilized post-fusion conformation form of the human metapneumovirus (hMPV) F protein or fragment thereof is of the A1 subgroup.
65. The composition of embodiment 63-64, wherein the composition consists essentially of i) a stabilized post-fusion conformation form of the human metapneumovirus (hMPV) F protein or fragment thereof of the A genotype and ii) a stabilized pre-fusion conformation form of the human metapneumovirus (hMPV) F protein or fragment thereof of the B genotype: and optionally one or more adjuvants and/or at least one pharmaceutically exactable carrier or excipient: wherein said immunogenic composition cross-neutralizes the other subgroup and/or other.
66. The composition of embodiment 65, wherein the stabilized post-fusion conformation form of the human metapneumovirus (hMPV) F protein or fragment thereof of the A genotype is of the A1 subgroup and wherein the stabilized post-fusion conformation form of the human metapneumovirus (hMPV) F protein or fragment thereof of the B genotype is of the B1 subgroup.
67. The immunogenic composition of any preceding embodiment, wherein the post-fusion F protein is the recombinant protein.
68. The immunogenic composition of any preceding embodiment, wherein the post-fusion F protein lacks the cytoplasmic tail and/or transmembrane domain.
69. The immunogenic composition of any preceding embodiment, wherein the post-fusion F protein has an amino acid sequence, which is a modified amino acid sequence of the native F protein derived from the hMPV strain or clinical isolate.
70. The immunogenic composition of embodiment 69, wherein the native F protein sequence is selected from the group consisting of the amino acid sequences of SEQ ID NO: 1 to 10 that are derived from the hMPV strains NL/1/00, NL/17/00, TN/94-49, NCL174, CAN97-83, NL/1/9, NDL00-1, C1-334, CAN97-82 and TN/89-515.
71. The immunogenic composition of any preceding embodiment, wherein the post-fusion F protein has the amino acid sequence SEQ ID NO: 21 (SF_A1_MFur) or SEQ ID NO: 22 (SF_B1_MFur).

### BRIEF DESCRIPTION OF THE DRAWINGS

FIG. 1 shows the schematic diagram of the native hMPV F protein with the indicated domains: F0-protein precursor: F1 and F2 domains: SP—signal peptide; FP—fusion peptide: HRA, HRB—Heptad Repeat domain A and B: TM—transmembrane domain; CYT—cytoplasmic tail: S—S—disulfide bond.

FIG. 2 shows three-dimensional structures (ribbon diagrams) of the F protein in (A) the pre-fusion conformation and (B) the post-fusion conformation.

FIG. 3 shows serum neutralization antibody titers in mice raised against the combination pre- and post-fusion F proteins comprising the antigen dose of (A) 0.6 μg, (B) 0.2 μg, (C) 0.02 μg per F protein. Please note that the combination of pre- and post-fusion F proteins contain double amount of antigen. It could be that the antibodies raised against the post-fusion F-proteins primarily cross-protect the pre-fusion format. Thus, addition of post-fusion format F proteins may not be necessary.

FIG. 4. Neutralization titers induced against F protein candidates (0.02 μg per antigen) derived from A1 or B1 subgroups, or combinations thereof (challenge with A2a subgroup) dose per F protein. Please note that the combination of pre- and post-fusion F proteins contain double amount of antigen. It could be that the antibodies raised against the post-fusion F-proteins primarily cross-protect the pre-fusion format. Thus, addition of post-fusion format F proteins may not be necessary.

FIG. 5. Protection of mice upon challenge with the hMPV A2a subgroup: (A) FFA, (B) RT-qPCR. Please note that the combination of pre- and post-fusion F proteins contain double amount of antigen. It could be that the antibodies raised against the post-fusion F-proteins primarily cross-protect the pre-fusion format. Thus, addition of post-fusion format F proteins may not be necessary.

FIG. 6. Neutralization titers induced against F protein candidates derived from A and B groups in pre- and post format (40, 120 and 400 ng per antigen) (A) MNA against hMPV A1 strain: (B) MNA against hMPV B1 strain. A dose response is observed for all groups in both assays. When mice were immunized with pre-fusion A1 candidates, there was a good neutralization and cross-neutralization against hMPV A1 (A) and B1 (B) strain respectively. The pre-fusion A1 candidates may also induce a higher immunogenicity and thus better neutralize and cross-neutralize. When immunized with pre-fusion B1 candidates, the cross-neutralization against hMPV A1 strain was less effective. Same observation can be done for the post-fusion candidates. However, it could be that the post-fusion candidates raise similar antibodies (i.e. neutralization antibodies against pre-fusion format and additional non neutralizing or primarily non neutralizing antibodies against the post-format parts). Thus, it is our interpretation at this stage that the pre-fusion format is probably still preferred.

FIG. 7. Neutralization and cross-neutralization. Overall, the neutralization titers with combinations pre-post are weaker than those obtains with a single candidate. As previously observed, the immunization with A1 candidates only seems to be more cross-neutralizing and/or raise higher immunogenicity. In that experiment, the best combination would be Pre+Post A1 or Pre B1+Post A1, but the neutralization titers are still lower than in the immunization with a single candidate.

FIG. 8. Adjuvant effect on induction of the hMPV neutralizing antibodies. Mice immunization with the vaccine L7-A1-23+sF-A1-MFur (0.2 μg per each antigen) formulated with different adjuvants or without adjuvant. No neutralizing antibodies were induced with the combination vaccine formulated without adjuvant. The combination vaccine formulated with the different adjuvants induced neutralizing antibodies. From this experiment all adjuvants tested are valuable options for formulation of a F protein based hMPV vaccine.

### DETAILED DESCRIPTION OF THE INVENTION

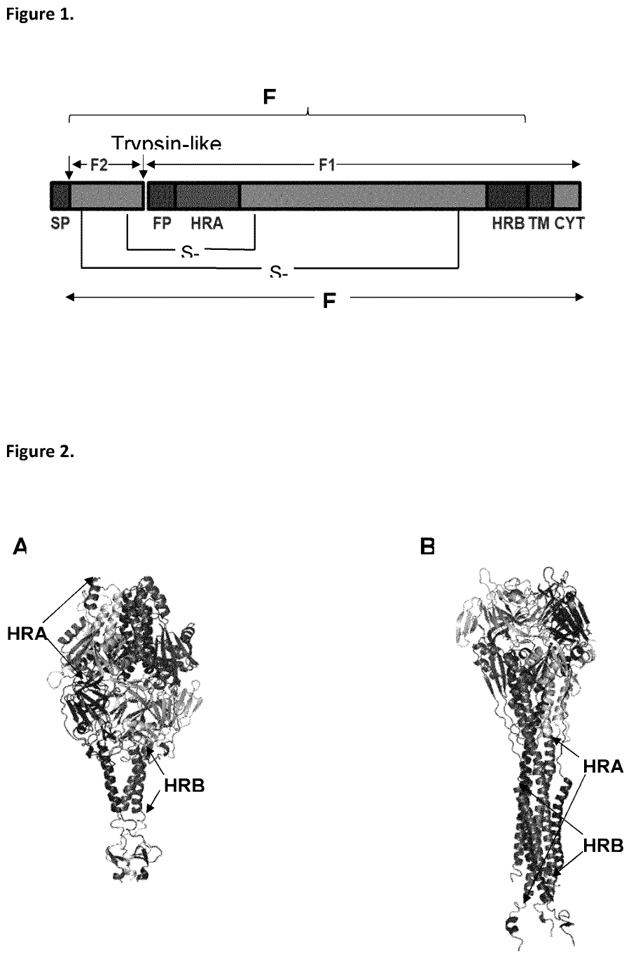

An object of the present invention is to provide an hMPV subunit vaccine for treating and/or preventing subjects against numerous hMPV strains. The subunit vaccine is based on a modified hMPV F protein stabilized in one of the pre-fusion conformation with various approaches of stabilization (see FIG. 1).

hMPV strains are classified into two genotypes: A and B, each divided into two subgroups A1, A2a, A2b, B1 and B2. The disclosed herein modified F proteins or fragments thereof can be derived from any hMPV strain or clinical isolate. Preferably, two F proteins in one composition (or vaccine) belong to different subgroups of the same genotype, even more preferably, to different genotypes. Examples of native F protein sequences derived from different strains are shown in Table 1.

TABLE 1
Exemplary native hMPV F proteins

| | Geno- | SEQ | |
|---|---|---|---|
| Strain | type | ID NO | F protein sequence |
| NL/1/00 | A1 | 1 | MSWKVVIIFSLLITPQHGLKESYLEESCSTITEGYLSVLRTGWYTNVFTLEVGDVENLTCADGPSLI |
| | | | KTELDLTKSALRELRTVSADQLAREEQIENPRQSRFVLGAIALGVATAAAVTAGVAIAKTIRLESE |
| | | | VTAIKNALKKTNEAVSTLGNGVRVLATAVRELKDFVSKNLTRAINKNKCDIADLKMAVSFSQFN |
| | | | RRFLNVVRQFSDNAGITPAISLDLMTDAELARAVSNMPTSAGQIKLMLENRAMVRRKGFGFLIGV |
| | | | YGSSVIYMVQLPIFGVIDTPCWIVKAAPSCSGKKGNYACLLREDQGWYCQNAGSTVYYPNEKDC |
| | | | ETRGDHVFCDTAAGINVAEQSKECNINISTTNYPCKVSTGRHPISMVALSPLGALVACYKGVSCSI |
| | | | GSNRVGIIKQLNKGCSYITNQDADTVTIDNTVYQLSKVEGEQHVIKGRPVSSSFDPVKFPEDQFNV |
| | | | ALDQVFESIENSQALVDQSNRILSSAEKGNTGFIIVIILIAVLGSTMILVSVFIIIKKTKKPTGAPPELS |
| | | | GVTNNGFIPHN |
| TN/94-49 | A2a | 2 | MSWKVVIIFSLLITPQHGLKESYLEESCSTITEGYLSVLRTGWYTNVFTLEVGDVENLTCADGPSLI |
| | | | KTELELTKSALRELKTVSADQLAREEQIENPRQSRFVLGAIALGVATAAAVTAGVAIAKTIRLESE |
| | | | VTAIKNALKKTNEAVSTLGNGVRVLATAVRELKDFVSKNLTRAINKNKCDIDDLKMAVSFSQFN |
| | | | RRFLNVVRQFSDNAGITPAISLDLMTDAELARAVSNMPTSAGQIKLMLENRAMVRRKGFGILIGV |
| | | | YGSSVIYMVQLPIFGVIDTPCWIVKAAPSCSKKKGNYACLLREDQGWYCQNAGSTVYYPNEKDC |
| | | | ETRGDHVFCDTAAGINVAEQSKECNINISTTNYPCKVSTGRHPISMVALSPLGALVACYKGVSCSI |
| | | | GSNRVGIIKQLNKGCSYITNQDADTVTIDNTVYQLSKVEGEQHVIKGRPVSSSFDPVKFPEDQFNV |
| | | | ALDQVFENIENSQALVDQSNRILSSAEKGNTGFIIVIILIAVLGSSMILVSIFIIIKKTKKQTGAPPELS |
| | | | GVTNNGFIPHS |
| NL/17/ | A2a | 3 | MSWKVVIIFSLLITPQHGLKESYLEESCSTITEGYLSVLRTGWYTNVFTLEVGDVENLTCSDGPSLI |
| 00 | | | KTELDLTKSALRELKTVSADQLAREEQIENPRQSRFVLGAIALGVATAAAVTAGVAIAKTIRLESE |
| | | | VTAIKNALKTTNEAVSTLGNGVRVLATAVRELKDFVSKNLTRAINKNKCDIDDLKMAVSFSQFN |
| | | | RRFLNVVRQFSDNAGITPAISLDLMTDAELARAVSNMPTSAGQIKLMLENRAMVRRKGFGILIGV |
| | | | YGSSVIYTVQLPIFGVIDTPCWIVKAAPSCSEKKGNYACLLREDQGWYCQNAGSTVYYPNEKDCE |
| | | | TRGDHVFCDTAAGINVAEQSKECNINISTTNYPCKVSTGRHPISMVALSPLGALVACYKGVSCSIG |
| | | | SNRVGIIKQLNKGCSYITNQDADTVTIDNTVYQLSKVEGEQHVIKGRPVSSSFDPIKFPEDQFNVA |
| | | | LDQVFENIENSQALVDQSNRILSSAEKGNTGFIIVIILIAVLGSSMILVSIFIIIKKTKKPTGAPPELSG |
| | | | VTNNGFIPHS |
| NCL174 | A2b | 4 | MSWKVVIIFSLLITPQHSLKESYLEESCSTITEGYLSVLRTGWYTNVFTLEVGDVENLTCADGPSLI |
| | | | KTELDLTKSALRELKPVSADQLAREEQIENPRQSRFVLGAIALGVATAAAVTAGVAIAKTIRLESE |
| | | | VTAIKNALKKTNEAVSTLGNGVRVLATAVRELKDFVSKNLTRAINKNKCDIDDLKMAVSFSQFN |
| | | | RRFLNVVRQFSDNAGITPAISLDLMTDAELARAVSNMPTAAGQIKLMLENRAMVRRKGFGILIGV |
| | | | YGSSVIYMVQLPIFGVIDTPCWIVKAAPSCSEKKGNYACLLREDQGWYCQNAGSTVYYPNEKDC |
| | | | ETRGDHVFCDTAAGINVAEQSKECNINISTTNYPCKVSTGRHPISMVALSPLGALVACYKGVSCSI |
| | | | GSNRVGIIKQLNKGCSYITNQDADTVTIDNTVYQLSKVEGEQHVIKGRPVSSSFDPVKFPEDQFNV |
| | | | ALDQVFENIENSQALVDQSNRILSSAEKGNTGFIIVIILIAVLGSSMILVSVFIIIKKTRKPTGAPPELS |
| | | | GVTNNGFIPHS |
| CAN97- | A2a | 5 | MSWKVVIIFSLLITPQHGLKESYLEESCSTITEGYLSVLRTGWYTNVFTLEVGDVENLTCSDGPSLI |
| 83 | | | KTELDLTKSALRELKTVSADQLAREEQIENPRQSRFVLGAIALGVATAAAVTAGVAIAKTIRLESE |
| | | | VTAIKNALKTTNEAVSTLGNGVRVLATAVRELKDFVSKNLTRAINKNKCDIDDLKMAVSFSQFN |
| | | | RRFLNVVRQFSDNAGITPAISLDLMTDAELARAVSNMPTSAGQIKLMLENRAMVRRKGFGILIGV |
| | | | YGSSVIYMVQLPIFGVIDTPCWIVKAAPSCSGKKGNYACLLREDQGWYCQNAGSTVYYPNEKDC |
| | | | ETRGDHVFCDTAAGINVAEQSKECNINISTTNYPCKVSTGRHPISMVALSPLGALVACYKGVSCSI |
| | | | GSNRVGIIKQLNKGCSYITNQDADTVTIDNTVYQLSKVEGEQHVIKGRPVSSSFDPIKFPEDQFNV |
| | | | ALDQVFENIENSQALVDQSNRILSSAEKGNTGFIIVIILIAVLGSSMILVSIFIIIKKTKKPTGAPPELS |
| | | | GVTNNGFIPHS |
| NL/1/99 | B1 | 6 | MSWKVMIIISLLITPQHGLKESYLEESCSTITEGYLSVLRTGWYTNVFTLEVGDVENLTCPSLIKTE |
| | | | LDLTKSALRELKTVSADQLAREEQIENPRQSRFVLGAIALGVATAAAVTAGIAIAKTIRLESEVNAI |
| | | | KGALKQTNEAVSTLGNGVRVLATAVRELKEFVSKNLTSAINRNKCDIADLKMAVSFSQFNRRFL |
| | | | NVVRQFSDNAGITPAISLDLMTDAELARAVSYMPTSAGQIKLMLENRAMVRRKGFGILIGVYGSS |
| | | | VIYMVQLPIFGVIDTPCWIIKAAPSCSEKNGNYACLLREDQGWYCKNAGSTVYYPNEKDCETRG |
| | | | DHVFCDTAAGINVAEQSRECNINISTTNYPCKVSTGRHPISMVALSPLGALVACYKGVSCSIGSNW |
| | | | VGIIKQLPKGCSYITNQDADTVTIDNTVYQLSKVEGEQHVIKGRPVSSSFDPIKFPEDQFNVALDQ |
| | | | VFESIENSQALVDQSNKILNSAEKGNTGFIIVVILVAVLGLTMISVSIIIIIKKTRKPTGAPPELNGVT |
| | | | NGGFIPHS |
| NDL00- | B1 | 7 | MSWKVVIIFSLLITPQHGLKESYLEESCSTITEGYLSVLRTGWYTNVFTLEVGDVENLTCADGPSLI |
| 11 | | | KTELDLTKSALRELRTVSADQLAREEQIENPRQSRFVLGAIALGVATAAAVTAGVAIAKTIRLESE |
| | | | VTAIKNALKKTNEAVSTLGNGVRVLATAVRELKDFVSKNLTRAINKNKCDIADLKMAVSFSQFN |
| | | | RRFLNVVRQFSDNAGITPAISLDLMTDAELARAVSNMPTSAGQIKLMLENRAMVRRKGFGFLIGV |
| | | | YGSSVIYMVQLPIFGVIDTPCWIVKAAPSCSGKKGNYACLLREDQGWYCQNAGSTVYYPNEKDC |
| | | | ETRGDHVFCDTAAGINVAEQSKECNINISTTNYPCKVSTGRHPISMVALSPLGALVACYKGVSCSI |
| | | | GSNRVGIIKQLNKGCSYITNQDADTVTIDNTVYQLSKVEGEQHVIKGRPVSSSFDPVKFPEDQFNV |
| | | | ALDQVFESIENSQALVDQSNRILSSAEKGNTGFIIVIILIAVLGSTMILVSVFIIIKKTKKPTGAPPELS |
| | | | GVTNNGFIPHN |
| CAN98- | B1 | 8 | MSWKVMIIISLLITPQHGLKESYLEESCSTITEGYLSVLRTGWYTNVFTLEVGDVENLTCTDGPSLI |
| 75 | | | KTELDLTKSALRELKTVSADQLAREEQIENPRQSRFVLGAIALGVATAAAVTAGIAIAKTIRLESE |
| | | | VNAIKGALKTTNEAVSTLGNGVRVLATAVRELKEFVSKNLTSAINKNKCDIADLKMAVSFSQFN |
| | | | RRFLNVVRQFSDNAGITPAISLDLMTDAELARAVSYMPTSAGQIKLMLENRAMVRRKGFGILIGV |
| | | | YGSSVIYMVQLPIFGVIDTPCWIIKAAPSCSEKDGNYACLLREDQGWYCKNAGSTVYYPNKKDCE |
| | | | TRGDHVFCDTAAGINVAEQSRECNINISTTNYPCKVSTGRHPISMVALSPLGALVACYKGVSCSIG |
| | | | SNRVGIIKQLPKGCSYITNQDADTVTIDNTVYQLSKVEGEQHVIKGRPVSSSFDPIKFPEDQFNVAL |
| | | | DQVFESIENSQALVDQSNKILNSAEKGNTGFIIVIILIAVLGLTMISVSIIIIIKKTRKPTGAPPELNGV |
| | | | TNGGFIPHS |
| C1-334 | B1 | 9 | MSWKVMIIISLLITPQHGLKESYLEESCSTITEGYLSVLRTGWYTNVFTLEVGDVENLTCTDGPSLI |
| | | | KTELDLTKSALRELKTVSADQLAREEQIENPRQSRFVLGAIALGVATAAAVTAGIAIAKTIRLESE |
| | | | VNAIKGALKQTNEAVSTLGNGVRVLATAVRELKEFVSKNLTSAINRNKCDIADLKMAVSFSQFN |
| | | | RRFLNVVRQFSDNAGITPAISLDLMTDAELARAVSYMPTSAGQIKLMLENRAMVRRKGFGILIGV |
| | | | YGSSVIYMVQLPIFGVIDTPCWIIKAAPSCSEKNGNYACLLREDQGWYCKNAGSTVYYPNEKDCE |
| | | | TRGDHVFCDTAAGINVAEQSRECNINISTTNYPCKVSTGRHPISMVALSPLGALVACYKGVSCSIG |
| | | | SNRVGIIKQLPKGCSYITNQDADTVTIDNTVYQLSKVEGEQHVIKGRPVSSSFDPIRFPEDQFNVAL |
| | | | DQVFESIENSQALVEQSNKILNSAEKGNTGFIIVIILVAVLGLTMISVSIIIIIKKTRKPTGAPPELNGV |
| | | | TNGGFIPHS |
| TN/89- | B2 | 10 | MSWKVMIIISLLITPQHGLKESYLEESCSTITEGYLSVLRTGWYTNVFTLEVGDVENLTCTDGPSLI |
| 515 | | | KTELDLTKSALRELKTVSADQLAREEQIENPRQSRFVLGAIALGVATAAAVTAGIAIAKTIRLESE |
| | | | VNAIKGALKTTNEAVSTLGNGVRVLATAVRELKEFVSKNLTSAINKNKCDIADLKMAVSFSQFN |
| | | | RRFLNVVRQFSDNAGITPAISLDLMTDAELARAVSYMPTSAGQIKLMLENRAMVRRKGFGILIGV |
| | | | YGSSVIYMVQLPIFGVIDTPCWIIKAAPSCSEKDGNYACLLREDQGWYCKNAGSTVYYPNKKDCE |
| | | | TRGDHVFCDTAAGINVAEQSRECNINISTTNYPCKVSTGRHPISMVALSPLGALVACYKGVSCSIG |
| | | | SNRVGIIKQLPKGCSYITNQDADTVTIDNTVYQLSKVEGEQHVIKGRPVSSSFDPIKFPEDQFNVAL |
| | | | DQVFESIENSQALVDQSNKILNSAEKGNTGFIIVIILIAVLGLTMISVSIIIIIKKTRKPTGAPPELNGV |
| | | | TNGGFIPHS |
| CAN-97- | B1 | 49 | MSWKVVIIISLLITPQHGLKESYLEESCSTITEGYLSVLRTGWYTNVFTLEVGDVENLTCTDGPSLIK |
| 82 | | | TELDLTKSALRELKTVSADQLAREEQIENPRQSRFVLGAIALGVATAAAVTAGIAIAKTIRLESEV |
| | | | NAIKGALKQTNEAVSTLGNGVRVLATAVRELKEFVSKNLTSAINRNKCDIADLKMAVSFSQFNRRFLN |
| | | | VVRQFSDNAGITPAISLDLMTDAELARAVSYMPTSAGQIKLMLENRAMVRRKGFGILIGVYGSSV |
| | | | IYMVQLPIFGVIDTPCWIIKAAPSCSEKNGNYACLLREDQGWYCKNAGSTVYYPNEKDCETRGDHVFCD |
| | | | TAAGINVAEQSRECNINISTTNYPCKVSTGRHPISMVALSPLGALVACYKGVSCSIGSNRVGIIKQLPKG |
| | | | CSYITNQDADTVTIDNTVYQLSKVEGEQHVIKGRPVSSSFDPIRFPEDQFNVALDQVFESIENSQALVD |
| | | | QSNKILNSAEKGNTGFIIVIILVAVLGLTMISVSIIIIIKKTRKPTGAPLELNGVTNGGFIPHS |

In one aspect, the present invention relates to a soluble F protein, which mediates fusion of the virus and cell membrane during the infection process. The F protein is an integral membrane protein that spans the viral membrane once and contains at the N-terminus a cleavable signal sequence and at the C-terminus a hydrophobic TM domain anchoring the protein in the membrane and a short cytoplasmic tail (see FIG. 1). The native F protein exists in two conformation forms: pre-fusion and post-fusion (see FIG. 2). Outside the cell, the viral F protein is in the unstable globular pre-fusion conformation, which refolds into the elongated post-fusion form upon contact with the cell membrane. Both F protein conformations are antigenic and share several epitopes, while some epitopes are unique for each conformation. It was previously shown that antibodies raised against the F protein are neutralizing and play important role in combating hMPV infection.

For producing F proteins in the stabilized pre-fusion conformations, native F proteins were modified by recombinant technology (gene engineering); and DNA constructs were expressed in recombinant hosts.

According to one embodiment, the recombinant pre-fusion F protein was produced as a single-chain polypeptide. The single-chain F polypeptide has amino acid sequence similar to the sequence of F ectodomain, but lacking the fusion peptide (FP), which spans the amino acid residues at positions 103-118 of the native F protein, in particular, the native F protein sequence of SEQ ID NO: 1 to 10 and 49. Additionally, the single-chain F polypeptide lacks a protease cleavage site between the F1 and F2 domains, which is eliminated by introducing a mutation, preferably, at position 102 relative to the amino acid sequence of the native F protein. More preferably, this mutation is a substitution of the arginine residue to glycine (R102G). Furthermore, the pre-fusion F protein comprises at least one additional amino acid modification (such as substitution, deletion or insertion), especially at least one substitution to cysteine. This additional cysteine residue could form a non-natural disulfide (S—S) bond with another cysteine residue that further stabilizes the pre-fusion conformation.

According to yet another embodiment, in the single-chain F protein the F1 and F2 domains are connected by a heterologous peptide linker, which replaces amino acids 103 to 118 of the native F protein. The linker comprises up to five amino acids including alanine, glycine and/or valine, and at least one cysteine. Preferably, the cysteine residue is at position that corresponds to position 103 of the native F protein. Most preferably, the linker has the sequence CGAGA or CGAGV, in which C is at position 103. This cysteine could form a disulfide bond with a cysteine residue of the F1 domain.

According to yet one embodiment, the cysteine residue could be introduced at
any one of positions 103-120 and any one of positions 335-345;
any one of positions 107-118 and any one of positions 335-342;
any one of positions 117-129 and any one of positions 256-261;
any one of positions 87-102 and any one of positions 117-127;
any one of positions 102-113 and any one of positions 117-127;
any one of positions 102-113 and any one of positions 87-102;
any one of positions 337-341 and any one of positions 421-426;
any one of positions 112-120 and any one of positions 424-432;
any one of positions 150-156 and any one of positions 392-400;
any one of positions 112-120 and any one of positions 370-377;
any one of positions 365-375 and any one of positions 455-465;
any one of positions 365-375 and any one of positions 105-115; or
any one of positions 60-70 and any one of positions 175-185,
wherein the positions corresponds to the amino acids of the native F protein sequence, in particular, the native F protein sequence of SEQ ID NO: 1 to 10 and 49.

According to yet one embodiment, the pre-fusion F protein comprises one or more substitution(s) at positions corresponding to positions 49, 51, 67, 80, 137, 147, 159, 160, 161, 166, 177, 258, 266, 480 and/or 481 relative to the amino acid positions of the native F protein sequence, in particular, the native F protein sequence of SEQ ID NO: 1 to 10. The preferred substitution is selected from the group consisting of T49M, E80N, I137W, A147V, A159V, T160F, A161M, I67L, I177L, F258I, S266D, I480C and/or L481C.

More preferably, the single-chain pre-fusion F protein comprises one of the following combinations:
N97Q, R102G and G294E;
N97Q, R102G, T160F, I177L and G294E;
N97Q, R102G, T49M, I67L, A161M, E80N, F258I and G294E;
N97Q, R102G, T49M, I67L, A161M, E51C, K166C, S266D, G294E, I480C and L481C; or
N97Q, R102G, T49M, A161M, I137W, A159V, A147V, I177L and G294E.

In some embodiments, the pre-fusion single-chain F protein may be selected from the group consisting of, but not limited to, the following protein constructs: L7F_A1_23 (SEQ ID NO: 11), L7F_B1_23 (SEQ ID NO: 12), L7F_A1_23.2 (SEQ ID NO: 13), L7F_B1_23.2 (SEQ ID NO: 14), sF_A1_K_L7 (SEQ ID NO: 15), L7F_A1_31 (SEQ ID NO: 16), L7F_A1_33 (SEQ ID NO: 17) and/or L7F_A1_4.2 (SEQ ID NO: 18).

In particular, the pre-fusion F protein comprises or consists of the amino acid sequence of SEQ ID NO: 11 (L7F_A1_23 construct). In particular, the pre-fusion F protein comprises or consists of the amino acid sequence of SEQ ID NO: 13 (L7F_A1_23.2 construct). In particular, the pre-fusion F protein comprises or consist of the amino acid sequence of SEQ ID NO: 15 (sF_A1_K_L7 construct). In particular, the pre-fusion F protein comprises or consists of the amino acid sequence of SEQ ID NO: 16 (L7F_A1_31 construct). In particular, the pre-fusion F protein comprises or consists of the amino acid sequence of SEQ ID NO: 17 (L7F_A1_33 construct). In particular, the pre-fusion F protein comprises or consists of the amino acid sequence of SEQ ID NO: 18 (construct L7F_A1_4.2 construct). In particular. the pre-fusion F protein comprises or consists of the amino acid sequence of SEQ ID NO: 12 (L7_B1_23 construct). In particular, the pre-fusion F protein comprises or consists of the amino acid sequence of SEQ ID NO: 14 (L7_B1_23.2 construct).

According to another embodiment, the pre-fusion F protein consists of two polypeptide chains, i.e. distinct F1 and F2 domains connected by two or more S—S bonds, further containing at least one stabilizing mutation. preferably in the F1 domain. Exemplary two-chain pre-fusion F protein is sF_A1_K-E294 construct (SEQ ID NO: 19) and sF_B1_K-E294 construct (SEQ ID NO: 20).

According to yet another embodiment, the second protein of the composition disclosed herein is a modified F protein stabilized in the pre-fusion conformation. The pre-fusion F protein contains one or more stabilizing mutation(s). In particular, the pre-fusion F protein comprises or consists of the amino acid sequence of SEQ ID NO: 11 (L7F_A1_23 construct). In particular, the pre-fusion F protein comprises or consists of the amino acid sequence of SEQ ID NO: 13 (L7F_A1_23.2 construct). In particular, the pre-fusion F protein comprises or consist of the amino acid sequence of SEQ ID NO: 15 (sF_A1_K_L7 construct). In particular, the pre-fusion F protein comprises or consists of the amino acid sequence of SEQ ID NO: 16 (L7F_A1_31 construct). In particular, the pre-fusion F protein comprises or consists of the amino acid sequence of SEQ ID NO: 17 (L7F_A1_33 construct). In particular, the pre-fusion F protein comprises or consists of the amino acid sequence of SEQ ID NO: 18 (construct L7F_A1_4.2 construct). In particular, the pre-fusion F protein comprises or consists of the amino acid sequence of SEQ ID NO: 12 (L7_B1_23 construct). In particular, the pre-fusion F protein comprises or consists of the amino acid sequence of SEQ ID NO: 14 (L7_B1_23.2 construct).

According to another embodiment, the pre-fusion F protein consists of two polypeptide chains, i.e. distinct F1 and F2 domains connected by two or more S—S bonds, further containing at least one stabilizing mutation, preferably in the F1 domain. Exemplary two-chain pre-fusion F protein is sF_A1_K-E294 construct (SEQ ID NO: 19) and sF_B1_K-E294 construct (SEQ ID NO: 20).

In yet another embodiment, the invention provides post-fusion F proteins compositions. Particularly, the stabilized post-fusion F protein comprises the deletion of the amino acid residues at positions 103 to 111, replacement of R102 by a linker KKRKRR and the substitution G294E relative to the amino acid positions of the native F protein of SEQ ID NO: 1 to 9. Examples of the post-fusion F protein constructs are sF_A1_Mfur (SEQ ID NO: 21) and sF_B1_Mfur (SEQ ID NO: 22). Alternatively, the post-fusion construct are sF_A2_Mfur and sF_B2_Mfur.

According to yet another embodiment, the pre-fusion F protein may comprise or consist of the amino acid sequence having at least 80%, 85%, 90%, 95%, 96%, 97%, 98% or 99% sequence identity to the amino acid sequence selected from the group consisting of the sequences of SEQ ID NO: 11 to 22, wherein the percentage sequence identity is determined over the full length of the parental sequence by using the Needleman-Wunsch algorithm (Needleman & Wunsch. (1970) A general method applicable to the search for similarities in the amino acid sequence of two proteins. J Mol. Biol. 48:443-453). Otherwise, the percent sequence identity is determined by dividing the number of matches by the length of the sequence set forth in the identified sequence, or by an articulated length (such as 100 consecutive nucleotides or amino acid residues from a sequence set forth in an identified sequence), followed by multiplying the resulting value by 100. Preferably, the percentage sequence identity is determined over the full length of the sequence. For example, a peptide sequence that has 1166 matches when aligned with a test sequence having 1554 amino acids is 75.0 percent identical to the test sequence (1166÷1554*100−75.0). The percentage value of sequence identity is rounded to the nearest tenth. For example, 75.11, 75.12, 75.13, and 75.14 are rounded down to 75.1, while 75.15, 75.16, 75.17, 75.18, and 75.19 are rounded up to 75.2. Homologs and variants of a protein are typically characterized by possession of at least about 75% sequence identity, counted over at least 50, 100, 150, 250, 500 amino acid residues of the reference sequence, over the full length of the reference sequence or over the full-length alignment with the reference amino acid sequence. Importantly, such homologous protein or protein variant possesses an immunogenicity and protective efficacy comparable to the immunogenicity and protective efficacy of the parental F protein having a sequence of any SEQ ID NO: 11 to 22, wherein comparable immunogenicity can be measured in ELISA (IC₅₀ value) and/or neutralization assay (PRNT₅₀ value) and the read out is within a +/−50% margin, preferably +/−40%, more preferably +/−30%, 20% or 10% margin.

In an additional embodiment, the pre-fusion F protein of the present invention does not possess a transmembrane domain and a cytoplasmic tail. Nevertheless, it can be produced as a homo- or hetero-trimer. Trimerization can occur due to the sequence spanning the residues 480-495 of the native F protomer, however, trimerization can be facilitated by introducing modification(s) in this region. One modification includes substitution of the vicinal residues I480 and L481 for cysteine that allows introduction of three disulfide bonds across the three protomers in the form of a covalent ring. Another modification is insertion of a trimerization helper, so called foldon domain. Addition of the trimerization helper supports formation of a stable and soluble protein trimer. Availability of cysteine rings in the foldon domain allows forming the disulfide bonds making covalent connection between three protomers. In one embodiment, the foldon domain has the sequence of SEQ ID NO: 23 derived from fibritin of T4 bacteriophage or a modified sequence that contains one or more N-glycosylation site(s) (motif NxT/S, wherein “x” any amino acid residue except proline) helping to hide hMPV non-specific epitope(s). Examples of such modified foldon sequences are of SEQ ID NO: 24 to 28. Alternatively, a variant of the foldon domain may contain structural elements from the GCN4 leucine zipper (Harbury et al. 1993. Science 262:1401) or monomers of self-assembling nanoparticles, e.g., ferritin or lumacine synthase. Additionally, a linker may be used in the combination with a cleavage site, introduced by e.g. replacement of A496 residue. Non-limiting examples of short linkers are: GG, SG, GS, GGG, GGA, GGS, SGG, SSG, SGS, SGA, GGA, SSA and SGGS.

In yet another embodiment, the foldon domain is attached to the C-terminus of the F protein replacing its transmembrane and cytosolic domains. In this case, the glycine residue at the N-terminus of the foldon domain is attached to the C-terminus of the F1 domain directly or via a peptide linker, which may include at least one protease site. For instance, the foldon domain can be attached via the “VSL” (SEQ ID NO: 29) or “VSA” (SEQ ID NO: 30) linker. Such linkers may be used in combinations with a protease cleavage site such as the thrombin cleavage site, TEV (Tobacco etch virus protease) or Factor Xa cleavage site. Such foldon may have the sequence of SEQ ID NO: 42 to 47.

In some embodiments, for easier purification of the recombinant protein the single-chain polypeptide may comprise any purification tag sequences known in the prior art. Examples of polypeptides that aid purification include, but are not limited to, a His-tag, a myc-tag, an S-peptide tag, a MBP-tag, a GST-tag, a FLAG-tag, a thioredoxin-tag, a GFP-tag, a BCCP, a calmodulin tag, a streptavidin-tag, an HSV-epitope tag, a V5-epitope tag and a CBP-tag. Preferably, the F proteins of the present invention comprise the His and/or streptavidin-tags.

In yet another embodiment, the present invention provides isolated nucleic acid molecules encoding the recombinant hMPV F proteins of SEQ ID NO: 11 to 22 disclosed herein. In one certain embodiment, the nucleic acids encoding the proteins of the present invention comprise or consist of the sequences of SEQ ID NO: 31 to 40. In another embodiment, the nucleic acid encoding the hMPV F proteins may include one or more modification(s), such as substitutions, deletions or insertions. In some embodiments, the present application also encompasses nucleic acid molecules encoding proteins having at least 85%, 90%, 95%, 96%, 97%, 98% or 99% sequence identity to the amino acid sequence of SEQ ID NO: 11 to 22. Preferably, the nucleic acid sequences exhibit between about 80 and 100% (or any value there between) sequence identity to polynucleotide sequences of SEQ ID NO: 31 to 40. Sequence identity can be determined by sequence alignment programs and parameters well known to those skilled in the art. Such tools include the BLAST suite for a local alignment (Altschul S.F., et al. 1997. “Gapped BLAST and PSI-BLAST: a new generation of protein database search programs”. Nucleic Acids Res. 25:3389-3402). A general global alignment can be performed by using the Needleman-Wunsch algorithm (Needleman & Wunsch. 1970. A general method applicable to the search for similarities in the amino acid sequence of two protein. J Mol. Biol. 48:443-453).

In a further embodiment, the nucleic acids described herein may include additional nucleotide sequences encoding segments that can be used to enhance the formation of protein trimers (so called foldon domains) or purification of expressed proteins (purification tags). In some embodiments, the nucleic acids disclosed herein may have codon-optimized sequences. The procedure, known as “codon optimization” is described e.g. in the U.S. Pat. No. 5,547.871. The degeneracy of the genetic code permits variations of the nucleotide sequences of the F proteins, while still producing a polypeptide having the identical amino acid sequence as the polypeptide encoded by the native polynucleotide sequence.

According to yet one embodiment, the pre- and post-fusion F proteins disclosed herein are recombinant proteins produced in a heterologous host cell. The production of the recombinant proteins may be achieved by any suitable methods, including but not limited to transient and/or stable expression of the protein-encoding sequences in a culture of the prokaryotic or eukaryotic cells. The protein-encoding (polynucleotide) constructs are conveniently prepared using standard recombinant techniques (see e.g. Sambrook et al., supra). Polynucleotide sequences encoding the proteins disclosed herein may be included in one or more vectors, which are introduced into a host cell where the recombinant proteins are expressed. Non-limiting examples of vectors that can be used to express sequences encoding the proteins of the present invention include viral-based vectors (e.g., retrovirus, adenovirus, alphavirus, baculovirus or vaccinia virus), plasmid vectors, yeast vectors, insect vectors, mammalian vectors or artificial vectors. Many suitable expression systems are commercially available. The expression vector typically contains coding sequence and expression control elements which allow expression of the coding sequence in a suitable host cell. The present invention provides expression systems designed to assist in expressing and providing the isolated polypeptides. The present application also provides host cells for expression of the recombinant hMPV proteins. In one embodiment, the host cell may be a prokaryote, e.g. E. coli. In another embodiment, the host cell may be a eukaryotic cell, e.g. selected from the group consisting of, but no limited to, EB66® (Valneva SE), Vero, MDCK, BHK, MRC-5, WI-38, HT1080, CHO, COS-7, HEK293, Jurkat, CEM, CEMX174, and myeloma cells (e.g., SB20 cells) (many these cell lines are available from the ATCC). A particularly preferred cell line for the production of the pre-fusion F proteins of the inventions is the CHO cell line. Cell lines expressing one or more above described protein(s) can readily be generated by stably integrating one or more expression vector(s) encoding the protein(s) under constitutive or inducible promoter. The selection of the appropriate growth conditions and medium is within the skill of the art.

Methods for producing the recombinant proteins disclosed herein or isolated nucleic acid (DNA or RNA) molecules encoding those proteins are incorporated into the present disclosure. In particular, methods for purifying the recombinant proteins are included. Non-limiting examples of suitable purification from the cell culture medium procedures include centrifugation and/or density gradient centrifugation (e.g. sucrose gradient), filtration, pelleting, and/or column or batch chromatography including ion-exchange, affinity, size exclusion and/or hydrophobic interaction chemistries, tangential filtration, etc. Such methods are known to those of skill in the art and are described in, e.g., Protein Purification Applications: A Practical Approach (E.L.V. Harris and S. Angal., Eds., 1990).

In a further embodiment, the F protein of the present invention may derive from any of the hMPV strain or clinical isolate belonging to either one of two genotype A and B, or subgroup A1, A2, B1 or B2.

In a further embodiment, the present invention provides the compositions comprising one F protein, especially the composition comprising the F protein existing in the pre-fusion conformation. In general, F proteins may be derived from any hMPV strain or clinical isolate. In one embodiment, the composition of the present invention comprises the F proteins derived from the genotype, A or B, i.e. subgroups A1 and A2a, A2b (alternatively, B1 and B2). In another preferred embodiment, the composition of the present invention comprises the F proteins derived from the subgroups A1, see also table 2.

In a further embodiment, the present invention provides the compositions comprising combinations of at least two F proteins, especially the compositions comprising F proteins existing in the pre-fusion conformations. In general, F proteins may be derived from any hMPV strain or clinical isolate. In one embodiment, the composition of the present invention comprises the F proteins derived from the same genotype, A or B, different subgroups, particularly subgroups A1 and A2a, A2b (alternatively, B1 and B2). In another embodiment, the composition of the present invention comprises the F proteins derived from the different genotypes A and B, for instance, subgroups A1 (or A2a, A2b) and B1 (or B2). Preferably it is a combination of an F protein of subgroup A1 with that of B1 or B2.

In one particular embodiment, the combination comprises the pre-fusion F proteins derived from the genotype A, particularly from the subgroup A1 or subgroup A2a, A2b, alternatively from both subgroups A1 and A2. In another embodiment, the combination comprises the pre-fusion F proteins derive from the genotype B, particularly from the subgroup B1 or subgroup B2, alternatively from both subgroups B1 and B2. In yet another embodiment, the combination comprises the pre-fusion F proteins from the different genotypes A and B. In particular, the pre-fusion F protein derives from the subgroup A1 (or A2a, A2b) and the pre-fusion F protein derives from the subgroup B1 (or B2). Alternatively, the pre-fusion F protein derives from the subgroup B1 (or B2) and the pre-fusion F protein derives from the subgroup A1 (or A2a, A2b). More specifically, the compositions that are parts of the present invention, which comprise the combination of the pre-fusion F proteins are cited in Table 2.

TABLE 2
Selected pre-fusion F proteins and combinations thereof

| | | | |
|---|---|---|---|
| A1pre | A1pre - | A1pre - | A1pre - |
| | A2a/A2bpre | B1pre | B2pre |
| A2pre - | A2pre | A2a/A2bpre - | A2a/A2bpre - |
| A1pre | | B1pre | B2pre |
| B1pre - | B1pre - | B1pre | B1pre - |
| A1pre | A2a/A2bpre | | B2pre |
| B2 pre- | B2 pre- | B2 pre- | B2pre |
| A1pre | A2a/A2bpre | B1pre | |

In a further embodiment, the immunogenic composition of the present invention is able to provide protection against more than one hMPV strain, particularly against strains that belong to different genotypes or different subgroups of one genotype. For instance, the immunogenic composition can provide protection against A1 and/or A2a, A2b subgroup(s), alternatively, against B1 and/or B2 subgroup(s), or against both A and B genotypes. Especially, cross-protection between A and B genotypes is desirable.

In a further embodiment, the present invention provides the pharmaceutical compositions comprising the combination of two recombinant F proteins available in the pre-fusion conformation forms. Typically, the pharmaceutical composition further comprises at least one pharmaceutically acceptable carrier or excipient. Pharmaceutically acceptable carrier is used to formulate the hMPV F protein for clinical administration. Remington's Pharmaceutical Sciences, by E. W. Martin, Mack Publishing Co., Easton, PA, 19th Edition, 1995, describes compositions and formulations suitable for pharmaceutical delivery of the immunogen. In general, the nature of the carrier depends on the particular mode of administration being employed. For instance, parenteral formulations usually comprise injectable and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions can contain minor amounts of non-toxic auxiliary substances, such as wetting or emulsifying agents, preservatives, and pH buffering agents and the like, for example sodium acetate or sorbitan monolaurate. In certain embodiments, the carrier suitable for administration to a subject is sterile, and/or suspended or otherwise contained in a unit dosage form containing one or more measured doses of the composition suitable to induce the desired anti-hMPV immune response. The unit dosage form may be, for example, in a sealed vial or a syringe for injection, or lyophilized for subsequent solubilization and administration or in a solid or controlled release dosage.

In some embodiments, the immunogenic composition (or vaccine) may further include an adjuvant. By adjuvant is meant any substance that is used to specifically or non-specifically potentiate an antigen-specific immune response, perhaps through activation of antigen presenting cells. Non-limiting examples of adjuvants include an aluminum salt (often referred to as “alum”) such as aluminium hydroxide or aluminium phosphate (as described in WO 2013/083726), an oil emulsion (such as complete or incomplete Freund's adjuvant), montanide Incomplete Seppic Adjuvant such as ISA51. a squalene-based oil-in-water emulsion adjuvants such as MF59® (Seqirus) (Ott G. et al. 1995. Pharm Biotechnol 6: 277-96), AddaVax™ (InvivoGen), monophosphoryl lipid A (MPL) (Cluff CW. 2010. Adv Exp Med Biol 667:111-23), Glucopyranosyl Lipid Adjuvant (GLA) (Coler RN et al. Development and characterization of synthetic glucopyranosyl lipid adjuvant system as a vaccine adjuvant, PLoS One. 2011, 6(1): e16333), toll like receptor ⅞ agonists such as 3M-052 (described in Zhao BG. et al. Combination therapy targeting toll like receptors 7, 8 and 9 eliminates large established tumors. J Immunother Cancer. 2014 May 13:2:12), polycationic peptide such as polyarginine (polyR) or a peptide containing at least two LysLeuLys motifs, especially KLKLLLLLKLK (described in WO 02/32451), immunostimulatory oligodeoxynucleotide containing non-methylated cytosine-guanine dinucleotides (CpG ODN), e.g., CpG 1018 (Dynavax) (e.g., as described in WO 96/02555) or ODNs based on inosine and cytidine (I-ODN) such as polyIC (e.g., as described in WO 01/93903), or deoxynucleic acid containing deoxy-inosine and/or deoxyuridine residues (as described in WO 02/95027), especially oligo(dIdC)₁₃ based adjuvant IC31® (Valneva SE) (as described in WO 2004/084938 and Olafsdottir et al. 2009. Scand J Immunol. 69(3): 194-202), neuroactive compound, especially human growth hormone (as described in WO 01/24822), a chemokine (e.g., defensins 1 or 2, RANTES, MIP1-α, MIP-2, interleukin-8, or a cytokine (e.g., interleukin-1β, −2, −6, −10 or −12; interferon-γ: tumor necrosis factor-α: or granulocyte-monocyte-colony stimulating factor), muramyl dipeptide (MDP) variants, non-toxic variants of bacterial toxins.

QS-21 (Antigenics Inc.), Quill A, MTP-PE and others as described in Sarkar et al. (2019), as well as adjuvant systems such as AF03, AS01, AS03 and AS04 (Giudice et al. 2018. Seminars in Immunology 39: 14-21). Usually. selection of a proper adjuvant depends on a type of B or T cell immune response desirable for a certain vaccine (Sarkar et al. (2019) Selection of adjuvants for vaccines targeting specific pathogens. Expert Rev Vaccines 18(5): 505 521). Generally, adjuvants that transduce immunological signals via TLR3, TLR4, TLR7, TLR8, and TLR9 receptors promotes Th1-biased immunity, while signaling via TLR2/TLR1, TLR2/TLR6 and TLR5 promotes Th2-biased immunity. For instance, such adjuvants as CpG ODN, polyIC and MPL predominantly induce Th1 responses, alum is a strong inducer of a Th2 response, while MF59®, AddaVax™, and IC31® induce mixed Th1 and Th2 responses. A preferred adjuvant useful in the vaccine of the present invention may be selected from, but not limited to, alum, CpG ODN such as CpG 1018 (Dynavax), polyIC, IC31® (Valneva), MF59® (Seqirus), AddaVax™, AS03 (GSK), AS01 (GSK) or QS21 (Pfizer) or combination(s) thereof. The aluminium adjuvant particularly useful in the current invention is an aluminium salt providing an aqueous immunogenic composition with less than 350 ppb heavy metal (such as Cu, Ni, W, Co, Os, Ru, Cd, Ag, Fc, V, Cr, Pb, Rb and Mo), especially less than 1.25 ppb copper (particularly, Cu⁺ or Cu²⁺), based on the weight of the aqueous immunogenic composition. In some embodiments, the aluminum adjuvant, especially the aluminium adjuvant comprising more than 1.25 ppb cooper or more than 350 ppb heavy metal, may be used in the combination with a radical quenching compound, such as L-methionine, present in a sufficient amount, particularly, in a concentration of at least 10 mmol/l in the immunogenic composition. In some embodiments, the immunogenic composition comprising the aluminum adjuvant may further comprise a reactive compound selected from the group consisting of a redox active compound, a radical building compound, a stabilizing compound and a combination of any thereof, especially wherein the reactive compound is selected from the group consisting of formaldehyde, ethanol, chloroform, trichloroethylene, acetone, triton X-100, triton X-114, deoxycholate, diethylpyrocarbonate, sulfite, Na₂S₂O₅, beta-propiolactone, polysorbate such as Tween 20®, Tween 80®, O₂, phenol, pluronic type copolymers, and a combination of any thereof. An adjuvant may be formulated together with an antigen in one immunogenic composition or may be administered separately either by the same route as that of the antigen or by a different route.

In some embodiments, the immunogenic composition (or vaccine) disclosed herein may include one or more additional antigen(s), preferably a viral protein derived from hMPV, such as another F protein or a different hMPV protein. Presumably, inclusion of an additional hMPV protein into the F protein-based vaccine can provide an improved (more balanced and robust) immune response. Among different hMPV proteins, the M protein has been described as such that is able to modulate humoral and cellular immune responses (especially Th1/Th2 balance), thereby providing an adjuvant effect in mice when the M protein is combined with the F protein (Aerts et al. 2015. Adjuvant effect of the human metapneumovirus (HMPV) matrix protein in HMPV subunit vaccines. J Gen Virol. 96 (Pt 4): 767-774). Therefore, in one embodiment, the immunogenic composition described herein includes the recombinant hMPV M protein for increasing protection conferred by the vaccine. The recombinant M protein may comprise the amino acid sequence of SEQ ID NO: 41 or a fragment thereof, or a variant thereof having at least 80% sequence identity to the parent M protein. Preferably, the recombinant M protein of the present invention consists of the amino acid sequence of SEQ ID NO: 41.

The additional hMPV protein may be the surface glycoprotein G or the small hydrophobic protein SH. Despite the fact that antibodies induced against the G and SH proteins do not protect against hMPV infection in animal models (Skidopoulus et al. (2006) Individual contributions of the human metapneumovirus F, G, and SH surface glycoproteins to the induction of neutralizing antibodies and protective immunity. Virology 345:492-501: Ryder et al., (2010) Soluble recombinant human metapneumovirus G protein is immunogenic but not protective. Vaccine 28(25): 4145 4152), one can suggest that these antigens could contribute to the protection in humans. Furthermore, high degree of genetic diversity between the A and B genotypes for these proteins could become important for immunoprophylaxis, such that both genotypes would need to be represented in a vaccine.

In some embodiments, the additional antigen may be derived from another virus causing a respiratory tract infection, such as RSV (Respiratory Syncytial Virus), PIV3 (ParaInfluenza Virus type 3), influenza virus or a coronavirus (such as SARS-CoV, SARS-CoV-2, MERS or alike). For instance, the additional antigen may be the RSV F protein, PIV3 F protein, influenza hemagglutinin or coronavirus S-protein. Such immunogenic compositions (vaccines) would be protective against more than one virus, representing combinatorial vaccines against respiratory tract infections.

In a further embodiment, the composition of the present invention is an immunogenic composition or vaccine comprising at least two immunogenic hMPV F proteins, especially the combination of two F proteins available in the pre-fusion conformations. Typically, the immunogenic composition or vaccine is capable of eliciting an antigen-specific immune response to an immunogenic protein(s). The immune response may be humoral, cellular, or both. A humoral response results in production of F protein-specific antibodies by the mammalian host upon exposure to the immunogenic composition. F protein-specific antibodies are produced by activated B cells. Production of neutralizing antibodies depends on activation of specific CD4+ T cells. In addition, there is evidence that protection against hMPV infection may employ CD8⁺ T cells (CTL response) that cooperate synergistically with CD4+ T cells (Kolli et al. (2008) T Lymphocytes Contribute to Antiviral Immunity and Pathogenesis in Experimental Human Metapneumovirus Infection. JOURNAL OF VIROLOGY, September 2008. p. 8560-8569). Therefore, the immunogenic composition or vaccine of the present invention induces a measurable B cell response (such as production of antibodies) against the hMPV F protein and/or a measurable CTL response against the hMPV virus when administered to a subject.

According to the present invention, the immunogenic composition is able to elicit antibodies directed against both conformations of the F protein: the pre-fusion fusion. Preferably, the anti-F protein antibodies are neutralizing antibodies able to interfere with the native F antigen existing in any (or both) conformation(s) and deactivate the virus. Most preferably, a neutralizing antibody response induced in the immunized subject is sufficient to combat an hMPV infection. A neutralizing antibody response may be measured in sera by ELISA and/or PRNT and/or MNA method or any other method known in the art.

Additionally, the immune response (e.g., neutralizing antibody titers) raised against the composition comprising two F proteins in the pre- and post-fusion conformations is superior to immune response (neutralizing antibody titers) elicited by the composition comprising a single (pre-) F protein used at the same amount as in the composition comprising the combination disclosed herein. Moreover, a synergistic effect from combining two immunogenic F proteins in one composition make the immunogenic composition (or vaccine) more potent than a single F protein composition (or vaccine) that may allow reducing a therapeutic or prophylactic dosage.

In one embodiment, the immunogenic composition or vaccine can reduce the severity of the symptoms associated with hMPV infection and/or decreases the viral load compared to a control in the subject upon administration. In another embodiment, the immunogenic composition or vaccine can reduce or prevent hMPV infection. In a preferred embodiment, the immunogenic composition or vaccine of the present invention can protect the immunized mammalian subject against hMPV infection.

Additionally, the immunogenic composition of the present invention is capable of providing protection against more than one hMPV strain, especially against different hMPV subgroups or genotypes. In one embodiment, the immunogenic composition can provide protection against viruses of the genotype A. In yet one embodiment, the immunogenic composition can provide protection against viruses of the genotype B. In a preferred embodiment, the immunogenic composition described herein is protective against both A and B genotypes. In particular embodiments, the immunogenic composition can provide protection against A1 and/or A2a/A2b subgroup(s), alternatively, against B1 and/or B2 subgroup(s), or against both A and B genotypes. In a preferred embodiment, cross-protection between the A and B genotypes is feasible.

In a further embodiment, the present invention includes combinations of the immunogenic composition or vaccine disclosed herein and a different hMPV vaccine or another respiratory vaccine, such as an anti-RSV, PIV3, influenza or coronavirus (such as SARS-CoV, SARS-CoV-2, MERS or alike) vaccine. Particularly, the combination may comprise the hMPV vaccine comprising the recombinant hMPV pre-/post-fusion F proteins and another subunit hMPV vaccine or an hMPV vaccine based on the whole virus or VLP particles. Additionally, the combination may comprise the recombinant hMPV F protein vaccine disclosed herein and an RSV vaccine, or the recombinant hMPV F protein vaccine and a PIV3 vaccine, or the recombinant hMPV F protein vaccine and an influenza vaccine, or the recombinant hMPV F protein vaccine and a coronavirus (especially, anti-SARS-CoV-2) vaccine. Preferably, the combination comprises the recombinant hMPV F protein vaccine disclosed herein and a recombinant RSV F protein vaccine. In one embodiment, the combination is understood as a combination of separate vaccine formulations administered simultaneously or subsequently by the same or different route. In another embodiment, two vaccines are combined in a single formulation.

In another embodiment, the immunogenic composition disclosed herein may be used as a medicament or vaccine, particularly in connection with a disease linked to or associated with hMPV infection, particularly for treating and/or preventing in a mammalian subject. Accordingly, the immunogenic composition (or vaccine) described herein is administered to a subject in a therapeutically effective amount. A therapeutically effective amount is the amount of a disclosed immunogen or immunogenic composition, that is sufficient to prevent, treat (including prophylaxis), reduce and/or ameliorate symptoms and/or underlying causes of a disorder or disease, for example to prevent, inhibit and/or treat hMPV infection. In some embodiments, a therapeutically effective amount is sufficient to reduce or eliminate a symptom of a disease, such as hMPV infection. For instance, this can be the amount necessary to inhibit or prevent viral replication or to measurably alter outward symptoms of the viral infection. In general, this amount will be sufficient to measurably inhibit virus replication or infectivity. Typically, a desired immune response inhibits, reduces or prevents hMPV infection. In one embodiment, the infection does not need to be completely eliminated, reduced or prevented for the method to be effective. For example, administration of a therapeutically effective amount of the agent can decrease the infection (as measured by infection of cells, or by number or percentage of infected subjects), for example by at least 10%, at least 20%, at least 50%, at least 60%, at least 70%, at least 80%, at least 90%, at least 95%, at least 98%, or even 100% as compared to a suitable control. In another embodiment, complete elimination or prevention of detectable hMPV infection is desirable. A further target indication is selected from the group consisting of mild respiratory disease. severe respiratory disease, hospitalization and/or death caused by the hMPV infection.

The pharmaceutical composition (or vaccine) disclosed herein may be administered by any means and route known to the skilled artisan. In some embodiments, the compositions (vaccines) may be formulated for parenteral administration by injection. As used herein, “parenteral” administration includes, without limitation, subcutaneous, intracutaneous, intravenous, intramuscular, intraarticular, intrathecal, or by infusion. In some embodiments, the compositions may be formulated for mucosal (intranasal or oral) administration. Formulations for injection may be presented in unit dosage form, e.g., in ampoules or in multi dose containers, with an added preservative.

It is understood, that to obtain a protective immune response against hMPV can require multiple administrations of the immunogenic composition. Thus, a therapeutically effective amount encompasses a fractional dose that contributes in combination with previous or subsequent administrations to attaining a protective immune response. For example, a therapeutically effective amount of an agent can be administered in a single dose, or in several doses, for example daily, during a course of treatment (such as a prime-boost vaccination regimen). However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

According to the present invention, dosage regimens have to be adjusted in order to provide the optimal desired response. In general, effective doses of the compositions disclosed herein for the prophylactic and/or therapeutic treatment may vary depending upon many different factors, including means of administration, target site, physiological state of the patient, age, whether the patient is human or non-human, other medications administered, whether treatment is prophylactic or therapeutic, etc. According to the present invention, the amount of the F protein in the unit dose may be anywhere in a broad range from about 0.01 μg to about 100 mg. Particularly, the composition of the invention may be administered in the amount ranging between about 1 μg and about 10 mg. especially between about 10 μg to about 1 mg. Preferably, the antigen formulation dosages need to be titrated to optimize safety and efficacy.

In a further embodiment, the present invention provides methods for generating anti-hMPV immune response in a subject that comprises administering a therapeutically effective amount of the immunogenic composition to the subject of need. The method includes stimulating B cells for producing F protein-specific antibodies and cytokine-producing T helper cells in order to protect said subject from hMPV infection or associated disease. In some cases, such method may comprise a prime-boost administration of the immunogenic composition. In other cases, such a method may comprise a boost administration of the immunogenic composition of the inventions. A booster effect refers to an increased immune response to the immunogenic composition upon subsequent exposure of the mammalian host to the same or alike immunogenic composition. For instance, the priming comprises administration of the composition with the F proteins of the genotype A, while the boosting comprises administration of the composition with the F proteins from the genotype B, and vice versa. Alternatively, the prime-boost immunization employs the same composition (homologous boosting), especially the mixed composition comprising the F proteins of both genotypes A and B.

In yet further embodiment, the present disclosure provides methods for treating and/or preventing an hMPV infection in the subjects, which comprise administering to the subjects a therapeutically effective amount of the immunogenic composition to generate neutralizing antibodies and provide protection against hMPV of one genotype, A or B, preferably against hMPV of both genotypes, A and B.

In yet further embodiment, the present disclosure provides methods for producing the pharmaceutical (immunogenic) compositions, including vaccines, employed in the invention. The method comprises i) expressing the recombinant pre- or post-fusion F protein from the corresponding nucleic acid molecule inserted in an expression vector in a host cell, ii) purifying the recombinant F protein: and iii) combining the purified recombinant protein with a pharmaceutically acceptable carrier and/or excipient, optionally with an adjuvant.

The pharmaceutical (immunogenic) compositions of the invention, including vaccines, can be produced in accordance with methods well known and routinely practiced in the art (see e.g., Remington: The Science and Practice of Pharmacy. Mack Publishing Co. 20th ed. 2000; and Ingredients of Vaccines—Fact Sheet from the Centers for Disease Control and Prevention, e.g., adjuvants, enhancers, preservatives, and stabilizers). The compositions disclosed herein are preferably manufactured under GMP conditions. The compositions of the invention, including vaccines, are formulated into pharmaceutically acceptable dosage forms by conventional methods known to those of skill in the art.

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The methods and techniques of the present disclosure are generally performed according to conventional methods well known in the art and as described in various general and more specific references that are cited and discussed throughout the present specification unless otherwise indicated. Although any methods and materials similar or equivalent to those described herein can be used in the practice of the present invention, the preferred methods, and materials are described herein.

The present invention is further illustrated by the following Examples, Figures, Tables and the Sequence listing, from which further features, embodiments and advantages may be taken, but which in no way should be construed as further limiting.

### EXAMPLES

### Example 1: Production of the Recombinant Pre- and Post-Fusion F Proteins

### Strains

The native hMPV F protein can be selected from any hMPV strain and any serotype represented by the sequences of SEQ ID NOs 1 to 10, or fragments, or variants thereof. In certain embodiments, the hMPV F protein derives from the strain NL/1/00, genotype A, subgroup A1, represented by SEQ ID NO: 1, the strain TN/94-49, genotype A, subgroup A2a, represented by SEQ ID NO: 2, the strain NCL174, genotype A, subgroup A2b, represented by SEQ ID NO: 4, the strain C1-334, genotype B, subgroup B1, represented by SEQ ID NO: 9 or the strain CAN97/82, genotype B, subgroup B1, represented by SEQ ID NO: 49, and the strain TN/98-515, genotype B, subgroup B2, represented by SEQ ID NO: 10.

### Expression Vectors

The plasmid pVVS 1371 used for cloning contains:
an HS4 insulator sequence from chicken β-globin locus,
two CMV promoters,
two chimeric introns, downstream of the CMV promoters, composed of the 5′-donor site from the first intron of the human β-globin gene and the branch and 3′-acceptor sites from the intron of an immunoglobulin gene heavy chain variable region. The sequences of the donor and acceptor sites, along with the branch point site, were adapted to match the consensus sequences for splicing. The intron is located upstream of the cDNA insert in order to prevent utilization of possible cryptic 5′-donor splice sites within the cDNA sequence,
the bovine growth hormone polyadenylation signal sequence (bGH A),
the neomycin phosphotransferase gene from Tn5 under the regulation of the SV40 enhancer and early promoter region,
the HSV TK polyadenylation signal of the thymidine kinase gene of herpes simplex virus is located downstream of the neomycin phosphotransferase gene,
a kanamycin resistance gene under the regulation of a bacterial promoter, and
a pUC origin of the replication.

The coding sequence of the wild type F protein was isolated from the hMPV strain NL/1/00, subgroup A1 and was codon-optimized for expression in CHO cells. The coding sequences of the wild type and modified F proteins were cloned into pVVS1371 plasmid for transient or stable protein expression in CHO cells.

Briefly, the coding sequences were cloned between the chimeric intron and the bGH a polyadenylation site of the pVVS1371 vector using the restriction sites SalI and PacI. The vector and the synthetized coding sequence (synthesis was done by GeneArt) were digested with SalI and PacI before purification on an agarose gel. The fragments were ligated with T4 DNA ligase and the ligation product was used to transform Max efficiency DH5α competent cells. Selected clones were tested for designed mutations by sequence analysis.

### Expression in CHO Cells

The protein expression was based on transient transfection of CHO cells using a MaxCyte® STX Scalable Transfection System device and following experimental recommendations of the supplier. Briefly, prior to electroporation, CHO cells were pelleted, suspended in MaxCyte® electroporation buffer and mixed with corresponding expression plasmid DNA. The cell-DNA mixture was transferred to a cassette processing assembly and loaded onto the MaxCyte® STX Scalable Transfection System. Cells were electroporated using the “CHO” protocol preloaded in the device and immediately transferred to culture flasks and incubated for 30 to 40 minutes at 37° C. with 8% CO₂. Following the recovery period, cells were resuspended at high density in EX-CELL ACF CHO medium (Sigma-Aldrich). Post-electroporation cell culture was carried out at 37° C., with 8% CO₂ and orbital shaking.

The production kinetics consist of decreasing the culture temperature to 32° C. and feeding the transfected cells daily with a fed-batch medium developed for transient protein expression in CHO cells (CHO CD EfficientFeed™ A (ThermoFischer Scientific), supplemented with yeastolate, glucose and glutaMax). After about 7 to 14 days of culture, cell viability was checked and conditioned medium was harvested after cell clarification corresponding to two runs of centrifugation at maximum speed for 10 minutes. Clarified product was filtered through a 0.22 μm sterile membrane and stored at −80° C. before protein purification.

### Protein Detection by Intracellular Immunostaining

At day 7 post transfection, cells were washed once in PBS and fixed for 10 minutes in 4% paraformaldehyde at room temperature. Fixed cells were permeabilized in BD Perm wash for 15 minutes at room temperature and incubated with the primary antibody diluted in BD Perm wash for 1 hour at 4° C. Finally, a secondary antibody coupled to a fluorescent marker was added for 1 hour at 4° C. and stored in PBS at 4° C. until analysis by flow cytometry (MacsQuant Analyzer, Miltenyi Biotec). As the primary antibody the MPE8 N113S antibody (PRO-2015-026-01) specifically recognizing the pre-fusion conformation of the hMPV F protein, or the DS7 IgG1 antibody (PRO-2016-003) recognizing both pre- and post-fusion hMPV F protein have been used. The fluorescent FITC-conjugated secondary antibody was goat anti-mouse IgG+IgM (JIR 115-096-068).

### Protein Purification

Frozen supernatant was brought to a room temperature and dialyzed with a standard grade regenerated cellulose dialysis membrane Spectra/Por®: 1-7 CR (MWCO: 3.5 kDa) (Spectrum) against PBS. Subsequently, it was equilibrated with 50 mM Na₂HPO₄ buffer at pH 8.0, 300 mM NaCl and purification of the protein was performed using Immobilized Metal ion Affinity Chromatography (IMAC) followed by gel filtration chromatography.

For IMAC, agarose resin containing Ni²⁺ (His GraviTrap) was packed into chromatography columns by the manufacturer (GE Healthcare). The resin was washed with two volumes of deionized water and equilibrated with three volumes of equilibration and wash buffer (20 mM sodium phosphate, pH 7.4, with 0.5 M sodium chloride and 20 mM imidazole) as indicated by the manufacturer. After sample loading the column was washed with 10 mL of wash buffer. The His-tagged protein was eluted from the column using 3-10 column volumes of elution buffer as indicated by the manufacturer (50 mM sodium phosphate, pH 8.0, with 0.5 M sodium chloride and 500 mM imidazole). Eluate was then filtered on a 0.22 μm filter and dialyzed twice in Slide-A-lyzer™ Dialysis cassettes against a storage buffer (50 mM Na₂HPO₄, 300 mM NaCl, 5 mM EDTA, pH 8.0) before being aliquoted and stored at −20° C. Analysis of the purity, size and aggregation of the recombinant proteins was performed by size exclusion chromatography (SE-HPLC) and SDS-PAGE SE-HPLC (Shimadzu) was run on the column SUPERDEX200 (GE Healthcare).

### Example 2: Conformation of the Recombinant hMPV F Proteins

### Determination of a Conformation Profile by Sandwich ELISA

Medium binging plates (Greiner) were coated with the human IgG1 DS7 capture antibody (Williams et al., 2007) at 200 ng/well and incubated overnight at 4° C. After 3× washing with water, the plates were saturated for 2 hours at 37° C. with PBS 0.05% Tween 20 and 5% dried-skimmed milk under agitation (saturation buffer). The liquid was removed from the wells and after 3× washing with water plates were incubated for 1 hour at 37° C. with 2.5 ng/well of the purified proteins of interest diluted in the saturation buffer. After washing, 5-fold serial dilution in saturation buffer of mouse antibody MPE8 N113S (Corti et al., 2013) directed against pre-fusion hMPV F protein or mouse antibody MF1 (Melero, personal communications) directed against post-fusion hMPV F protein were incubated for 1 hour at 37° C. Then the immune complexes were detected by incubation for one hour at 37° C. with secondary α-Ig species-specific antibody conjugated with peroxidase HRP Goat Anti-Mouse IgG (Covalab # lab0252) followed by 50 μL of peroxidase substrate (TMB, Sigma). The colorimetric reaction was stopped by adding 3 N H₂SO₄ and the absorbance of each well was measured at 490 nm with a spectrophotometer (MultiSkan).

### Example 3: Immunogenicity Study

### Immunogenicity in Mice

Groups of five to ten BALB/c mice were immunized three times with two or three weeks interval (e.g. days 0, 14 or 21 and 28 or 42) subcutaneously with the recombinant pre- and post- fusion F proteins used alone or in different combinations in amounts from 0.02 to 6.0 μg per mouse with or without adjuvants. One to four weeks after the last immunization, blood was drawn by retro-orbital bleeding and sera were prepared. Evaluation of the immune response was performed by indirect ELISA as described below.

### Subclass IgG ELISA

The recombinant F protein is diluted in carbonate/bicarbonate buffer at pH 9.6, and 50 ng of the protein per well was added to 96-well high binding plate (50 μL/well, Greiner). The plates were incubated overnight at 4° C. The wells were saturated for 30 minutes at room temperature with 150 μL of PBS 0.05% Tween 20 and 5% dried skimmed milk (saturation buffer). The liquid was removed from the wells and plates were incubated for 1 hour at room temperature with 50 μL/well of the sera of immunized mice at different dilutions (5-fold serial dilution) in saturation buffer. After washing 3 times with PBS 0.05% Tween 20, the immune complexes were detected by incubation for one hour at room temperature with 50 μl of secondary anti-IgG₁ or IgG₂ₐ mouse-specific antibody conjugated with peroxidase followed by 50 μL of peroxidase substrate (TMB, Sigma). The colorimetric reaction was stopped by adding orthophosphoric acid and the absorbance of each well was measured at 490 nm with a spectrophotometer (MultiSkan). As a read out, IC₅₀ values were calculated for evaluating specific antibody titers.

### Example 4: Induction of Neutralizing Antibodies

### Neutralization Assay

Briefly, the microneutralization assay (MNA) was used to determine a serum/antibody titer of an immunized subject required to reduce the number of hMPV virus plaques by 50% (MNA₅₀) as compared to a control serum/antibody. The MNA₅₀ was carried out by using monolayers of cells that can be infected with hMPV. Sera from subjects were diluted and incubated with the live hMPV virus. Virus infection was determined using an HRP-conjugated anti-F protein specific monoclonal antibody. A threshold of neutralizing antibodies of 1:10 dilution of serum in a PRNT₅₀/MNA₅₀ was generally accepted as evidence of protection (Hombach et. al. 2005. Vaccine 23: 5205-5211). Neutralizing antibodies provides the best evidence that protective immunity has been established, and the biological assay of neutralization shows correlation with protection (Hombach et al., 2005).

### Immunization and Challenge Protocol

The hMPV virus of A1 (strain NL/1/00), A2 (strain TN/94-49), B1 (strain C1-334) or B2 (strain TN/89-515) subgroup, propagated in LLC-MK2 cells (ATCC CCL-7) as described previously (Williams et al. 2005. The cotton rat (Sigmodon hispidus) is a permissive small animal model of human metapneumovirus infection, pathogenesis, and protective immunity. Journal of virology 79:10944-10951), were used in animal challenge experiments.

BALB/c mice were immunized three times with two weeks interval with adjuvanted recombinant F protein, as described previously, two weeks post-immunization they are challenged intranasally with around 1×10⁶ pfu of the hMPV. Four to five days later, the animals were sacrificed and individual serum samples were taken and frozen. Lung tissue samples were harvested, weighed and homogenized in 1 mL medium for determination of viral load. Viral load in lung tissues was determined by virus foci immunostaining, as described below. Alternatively or additionally, RT-qPCR was used to determine viral load in the lungs.

### MNA Protocol

On day −1, LLC MK2 cells, which were grown in OptiMEM containing 2% fetal bovine serum (FBS) and 1% antibiotic-antimycotic (Anti-Anti), were seeded into flat-bottom 96-well plates with a density of 2×10⁵ cells/mL (100 μL/well) and incubated at 37° C./5% CO₂ overnight. On day 0, the serum samples were diluted in OptiMEM containing 100 μM CaCl₂ and 1% Anti-Anti in U-bottom 96-well plates. As the sample dilutions are 1:1 mixed with the virus afterwards, 2× concentrated dilutions should be prepared. In control wells, without virus, medium was added instead of 2× concentrated virus dilution. The dilutions of the hMPV A1 virus, which is a trypsin-independent strain, were prepared in OptiMEM containing 100 μM CaCl₂ and 1% Anti-Anti in U-bottom 96-well plates according to the experimental setup. As the virus dilutions were 1:1 mixed with the diluted serum samples afterwards, the virus samples were prepared 2× concentrated (e.g. 120 pfu/60 μL). Blank wells are filled with medium. For the hMPV B1 virus and all other trypsin-dependent hMPV strains, tryspin (i.e. TrypLE) is added to the medium to help the infection, ranging from 8 to 50 mrPu/mL according to serum concentration. For the neutralization an equal volume (60 μL) of serum dilution and virus dilution was mixed (final concentration: 120 pfu/120 μL) and samples were incubated at room temperature for one hour. The flat-bottom 96-well plates containing the LLC MK2 cells were washed once with 150 μL/well PBS. After removal of the PBS, 100 μL of the pre-incubated serum:virus mix were transferred to plate with LLC MK2 cells and incubated at 37° C./5% CO₂ for five days. On day 5, 150 μL neutral-buffered Formalin solution was added per well and the plates were incubated at room temperature for 1 hour. The plates were washed twice with 300 L/well PBS and aspirated. 100 μL/well permeabilization buffer (PBS containing 0.5% Tween® 20) are added and the plates were incubated at 4° C. for 30 minutes. After aspiration of the permeabilization buffer, 100 μL/well blocking buffer (PBS containing 0.5% Tween® 20 and 10% skim milk) were added and the plates are incubated at 4° C. for 1 hour. A HRP-conjugated antibody (DS7 mIgG2a) was diluted in blocking buffer (see above) to a concentration of 0.5 μg/mL and after aspiration of the blocking buffer 50 μL of the antibody solution are added per well. The plates were then incubated at 37° C./5% CO₂ for one hour followed by washing six times with 200 μL/ well PBS using an ELISA washer. 100 μL TMB substrate were added per well and incubated at RT for approximately 10 minutes. The reaction was stopped with 50 μL 1 M sulfuric acid per well and the absorbance is measured at 450 nm.

For studying the pre/post-fusion F protein combinations, the pre-fusion L7F_A1_23 or L7F_B1_23 and the post-fusion sF_A1_Mfur or sF_B1_Mfur candidates were selected. The following compositions (combinations) of the pre- and post-fusion F proteins were tested for induction of hMPV neutralizing antibodies (see Table 3):

TABLE 3

| Confirmation | Composition 1 | Composition 2 | Composition 3 | Composition 4 |
|---|---|---|---|---|
| Pre-fusion | L7F_A1_23-His | L7F_A1_23-His | L7F_B1_23-His | L7F_B1_23-His |
| Post-fusion | sF_A1_MFur-His | sF_B1_MFur-His | sF_A1_MFur-His | sF_B1_MFur-His |

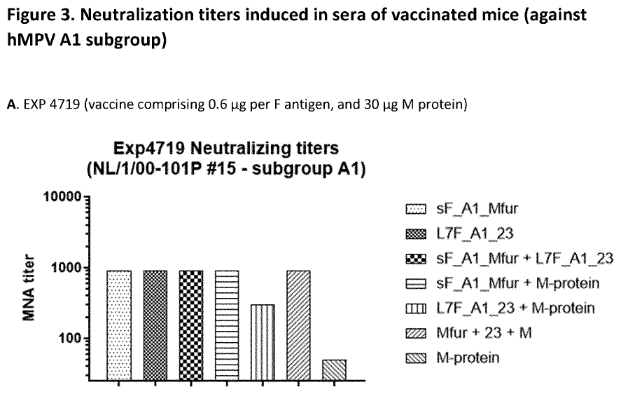

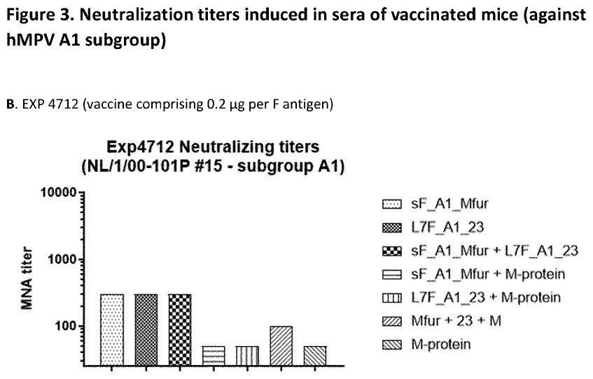

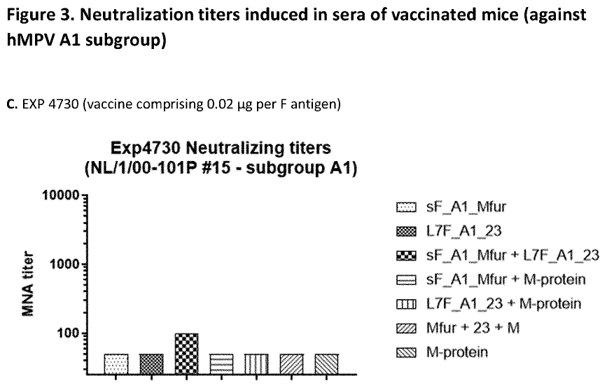

In six experiments performed in mice (see Table 4), each mouse was immunized either with the single F protein or with the combination vaccine. Mouse sera were used for testing neutralizing antibody titers performed by micro-neutralization assay (MNA) as described above. The results of these experiments are demonstrated in FIGS. 3 (A-C), 4 and 6 (A).

TABLE 4

| Vaccine | Antigen(s) | Exp4712 | | Exp4719 | | Exp4730 | | Exp4736 | Exp4763 | Exp4775 |
|---|---|---|---|---|---|---|---|---|---|---|
| Vaccine 1 | L7F_A1_23-His | 0.2 | μg | 0.6 | μg | 0.02 | μg | 0.02 μg | 0.04 μg or | — |
| | | | | | | | | | 0.12 μg or | |
| | | | | | | | | | 0.40 μg | |
| Vaccine 2 | sF_A1_MFur-His | 0.2 | μg | 0.6 | μg | 0.02 | μg | 0.02 μg | 0.04 μg or | — |
| | | | | | | | | | 0.12 μg or | |
| | | | | | | | | | 0.40 μg | |
| Vaccine 3 | L7F_A1_23-His | 0.2 μg + | | 0.6 μg + | | 0.02 μg + | | 0.02 μg + | | 0.04 μg or |
| | sF_A1_MFur-His | 0.2 μg | | 0.6 μg | | 0.02 μg | | 0.02 μg | | 0.12 μg or |
| | | | | | | | | | | 0.40 μg |
| Vaccine 4 | L7F_A1_23-His | 0.2 μg + | | 0.6 μg + | | 0.02 μg + | | — | — | — |
| | M-protein | 30 μg | | 30 μg | | 30 μg | | | | |
| Vaccine 5 | sF_A1_MFur-His | 0.2 μg + | | 0.6 μg + | | 0.02 μg | | — | — | — |
| | M-protein | 30 μg | | 30 μg | | 30 μg | | | | |
| Vaccine 6 | L7F_A1_23-His | 0.2 μg + | | 0.6 μg + | | 0.02 μg + | | — | — | — |
| | sF_A1_MFur-His | 0.2 μg + | | 0.6 μg + | | 0.02 μg | | | | |
| | M-protein | 30 μg | | 30 μg | | 30 μg | | | | |
| Vaccine 7 | M-protein | 30 | μg | 30 | μg | 30 | μg | — | | |

| | | | | | | | |
|---|---|---|---|---|---|---|---|
| Vaccine 8 | L7F_A1_23-His | — | — | — | 0.02 μg + | — | 0.04 μg or |
| | sF_B1_MFur-His | | | | 0.02 μg | | 0.12 μg or |
| | | | | | | | 0.40 μg |
| Vaccine 9 | L7F_B1_23-His | — | — | — | 0.02 μg + | — | 0.04 μg or |
| | sF_A1_MFur-His | | | | 0.02 μg | | 0.12 μg or |
| | | | | | | | 0.40 μg |
| Vaccine 10 | L7F_B1_23-His | — | — | — | 0.02 μg + | — | 0.04 μg or |
| | sF_B1_MFur-His | | | | 0.02 μg | | 0.12 μg or |
| | | | | | | | 0.40 μg |
| Vaccine 11 | L7F_B1_23-His | — | — | — | — | 0.04 μg or | — |
| | | | | | | 0.12 μg or | |
| | | | | | | 0.40 μg | |
| Vaccine 12 | sF_B1_MFur-His | — | — | — | — | 0.04 μg or | — |
| | | | | | | 0.12 μg or | |
| | | | | | | 0.40 μg | |

* For these experiments the amount of total protein used for vaccination is shown

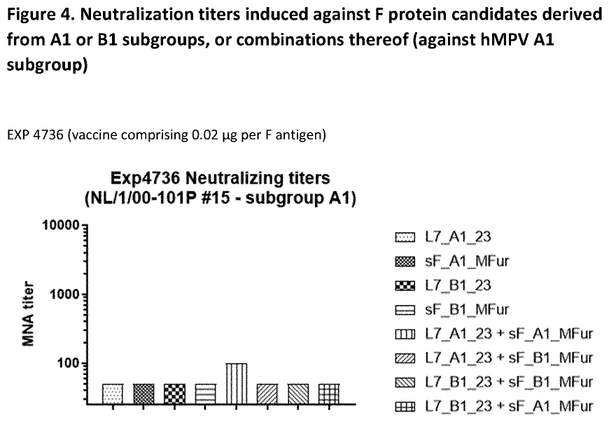

The data shown in FIGS. 3 and 4 demonstrate that the combination of the pre-fusion construct L7F_A1_23 and the post-fusion construct sF_A1_Mfur used at the amount of 0.02 μg per antigen per dose showed approximately 5-fold improvement of neutralization titer as compere to the single F protein (see FIGS. 3C and 4). At the higher antigen doses of 0.2 μg and 0.6 μg the synergistic effect of the combined pre-and post-fusion F proteins is not so pronounced (see FIG. 3A & B). From these experiments, it is also evident that the combination of two F proteins from A1 subgroup is protective against the challenge with the virus of the same genotype A, in particular A2 subgroup.

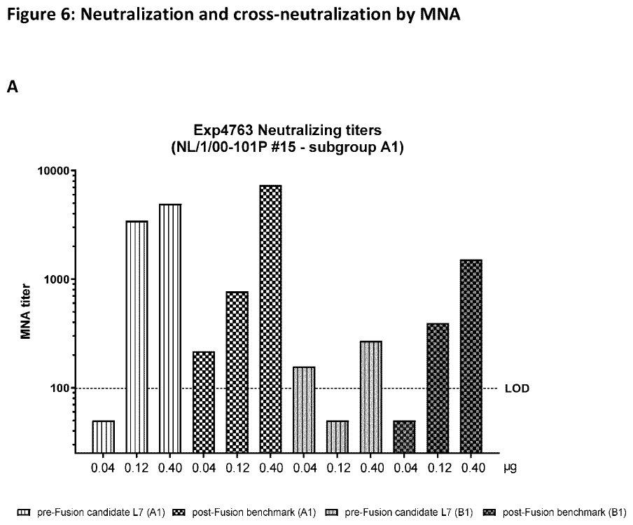

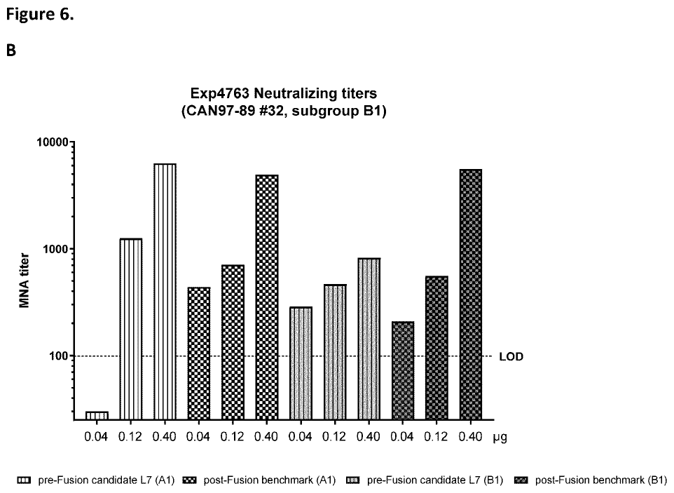

The data shown in FIG. 6 (experiment 4763) demonstrated a dose response for all groups in both assays. There was a good neutralization and cross-neutralization against hMPV A1 (A panel) and B1 (B panel), when mice were immunized with pre-fusion A1 candidates, whereas when immunized with pre-fusion B1 candidates, the cross-neutralization against hMPV A1 strain is less effective. The pre-fusion A1 candidate might also induce higher immunogenicity.

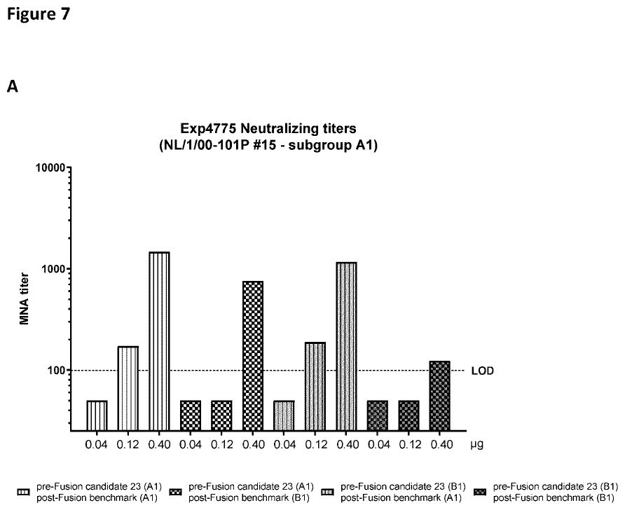

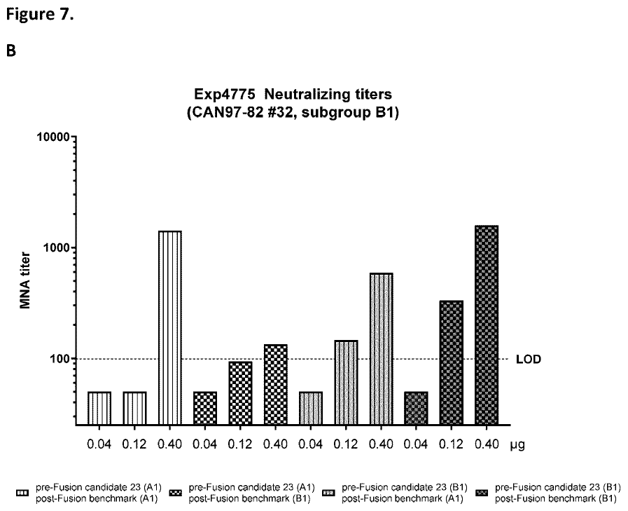

The data shown in FIG. 7 (experiment 4775) demonstrated overall, that the neutralization titers with the combination candidates were weaker than those obtained with a single candidate. As previously observed, the immunization with the A1 candidates only seems to be more cross-neutralizing (very weak neutralization against A1 for the mice immunized with B1 candidates). In this experiment, the best combination would be pre-post A1, again suggesting the A1 candidate to be preferred for cross-neutralization. However as mentioned, the neutralization titers were still lower than in the immunization with a single candidate.

### Example 5: Protection in Mice

Protection of mice upon immunization with the different pre-/post-fusion F protein compositions was evaluated in a mouse lung infection model.

### Immunization and Challenge Protocol

BALB/c mice are immunized three times with two weeks interval with adjuvanted recombinant F protein, as described previously, two weeks post-immunization they are challenged intranasally with around 1×10⁶ pfu of the hMPV. Four to five days later, the animals are sacrificed and lungs are taken and frozen. Lung tissue samples are harvested, weighed and homogenized in 1 mL medium for determination of viral load. Viral load in lung tissues is determined by virus foci immunostaining, as described below. Additionally, RT-qPCR is used to determine a viral load in the lungs.

### Virus Plaque (Foci) Immunostaining

The assay for hMPV foci quantification was developed based on the methods published in Williams et al., 2005. J Virology 79(17): 10944-51: Williams et al., 2007. J Virology 81(15): 8315-24: and Cox et al., 2012. J. Virology 86(22): 12148-60. Briefly, confluent cultures of Vero cells or LLC-MK2 cells in 24-well plates are infected with 125 μL/well of lung homogenate diluted in medium. After 1 hour incubation at 37° C./5% CO₂, overlay containing 1.5% methylcellulose in medium is added. At day 6 post-infection, the supernatant is removed and the cells are washed twice with PBS. Cell monolayers are fixed and stained with the DS7 antibody (mouse IgG₂ₐ). Foci are counted and cell images are captured with a Zeiss microscope using a 2.5× or 10× objective or using a BioReader 6000. Results of the immunostaining are expressed as focus forming units per milliliter, or FFU/mL.

### RT-qPCR Protocol

RNA is extracted from 140 μL lungs homogenates using the QIAamp Viral RNA Mini Kit following the manufacturer's instruction and the RNA is eluted in 60 μL. RT-qPCR is performed using the iTaq™ Universal Probes One-Step Kit (Bio-Rad). For amplification of the N gene the following primers (e.g. forward 5′-CATATAAGCATGCTATATTAAAAGAGTCTC-3′ and reverse 5′-CCTATTTCTGCAGCATATTTGTAATCAG-3′) and probe (e.g. FAM-TGYAATGATGAGGGTGTCACTGCGGTTG-BHQ1) are used. The reaction volume for RT-qPCR is 20 μL using 400 nM of each primer, 200 nM probe and 4 μL RNA. Revers transcription and amplification is performed using the CFX96 Touch Deep Well Real-Time PCR System (Bio-Rad) with the conditions listed in Table 5.

TABLE 5

| Step # | T ° C. | Time | | Activity |
|---|---|---|---|---|
| 1 | 50 | 10 | min | reverse transcription |
| 2 | 95 | 60 | s | inactivation/activation |
| 3 | 95 | 10 | s | denaturation |
| 4 | 57 | 30 | s | annealing/extension |
| 5 | cycle | 44 | times | cycling between 3 & 4 |

The amount of hMPV RNA is calculated to a known full-length hMPV RNA standard with known concentration included in each run using the program Bio-Rad CFX maestro.

As used herein, clearance or reduction of hMPV infection may be determined by any method known in the art. In some embodiments, a level of hMPV infection in the subject is determined, for example, by detecting the presence of the virus by real time reverse transcription quantitative polymerase chain reaction (RT-qPCR).

The first question addressed in this study is to compare protection efficacy after vaccination with the composition comprising the recombinant single F protein used either in the pre-fusion or post-fusion forms vs. a composition comprising the combination of pre- and post-fusion F proteins. The second addressed question is to evaluate the optimal antigen dose of the composition containing the combination of the pre-/post-fusion F proteins. The third question to be addressed herein is establishing a cross-protection between different hMPV genotypes and/or subgroups.

To assess protection efficacy, mice immunized with any composition shown in Table 4 were challenged with the strain TN/94-49 (A2 subgroup) or C1-334 (B1 subgroup).

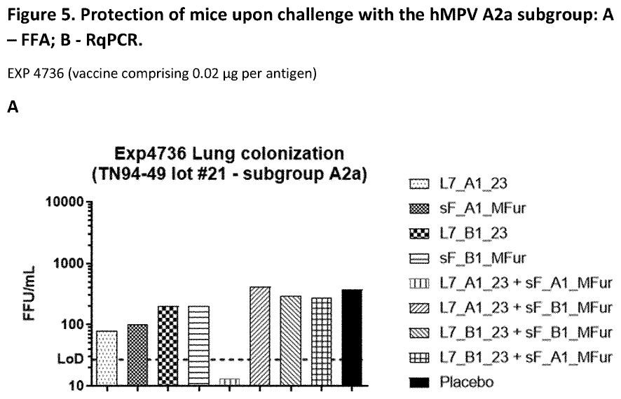

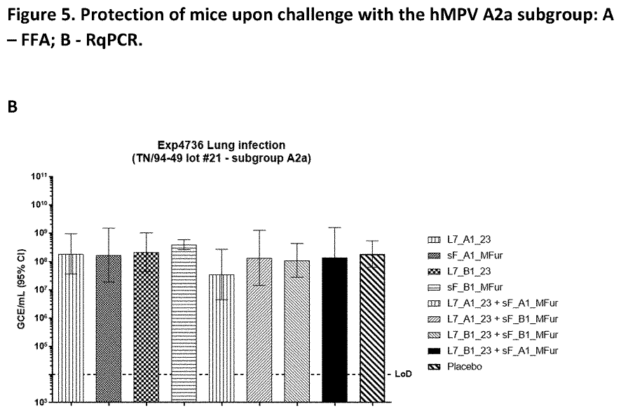

To evaluate a level of protection, the lung infection was assessed by FFA and RT-qPCR methods. The results are shown in FIGS. 5 (A, B), 6 (B, C) and 7 (A, B). In particular, FIG. 5A demonstrates that lowest level of foci indicating lung infection occurs in mice immunized with the combination of the pre- and post-fusion F proteins from A1 subgroup and challenged with the A2 strain.

Unfortunately, no such pronounced effect was demonstrated when PT-qPCR method was used (see FIG. 5B), most likely while FFA measures live virus and RT-qPCR viral RNA (live and dead virus) which can be detectable even in the absence of live virus at a time point when dead virus is not yet cleared from the lungs.

The different combinations shown in Table 4 at doses of 40 ng, 120 ng and 400 ng were tested in challenge experiments with either A1, A2 strain or B1 strain, and the results are demonstrated in FIGS. 6 (B, C) and 7 (A, B). Very similar results are seen between the different combinations in terms of lung infection by TN94-49 (subgroup A2) and C1-334 (subgroup B1) determined by focus forming assay (FIG. 6B & 7B) and RT-qPCR (data not shown).

### Example 6: Adjuvanticity Effect

BALB/c mice were immunized three times with two weeks interval with adjuvanted recombinant F protein vaccine, as described previously. Two weeks after the last immunization, blood was drawn by retro-orbital bleeding and sera were prepared. Evaluation of the immune response was performed by micro-neutralization assay (MNA) as described above.

For studying an adjuvanticity effect on the efficacy of the hMPV vaccine, one exemplary combination of the pre-/post-fusion F proteins L7F_A1_23 and sF_A1_Mfur was tested.

TABLE 5

| | | | Antigen |
|---|---|---|---|
| Adjuvant | Adjuvant amount, | Antigens | dose* |
| none | — | L7F_A1_23-His | 0.2 μg |
| | | sF_A1_MFur-His | 0.2 μg |
| alum | 0.1% | L7F_A1_23-His | 0.2 μg |
| | | sF_A1_MFur-His | 0.2 μg |
| alum + MPL | 0.1% + 5 μg | L7F_A1_23-His | 0.2 μg |
| | | sF_A1_MFur-His | 0.2 μg |
| IC31_{high} | 10 nmol KLK/0.4 | L7F_A1_23-His | 0.2 μg |
| | nmol ODN1a | sF_A1_MFur-His | 0.2 μg |
| IC31_{high} + alum | 10 nmol KLK/0.4 | L7F_A1_23-His | 0.2 μg |
| | nmol ODN1a + 0.1% | sF_A1_MFur-His | 0.2 μg |
| 3M-052-Alum | 1 μg 3M-052/100 μg | L7F_A1_23-His | 0.2 μg |
| | alum | sF_A1_MFur-His | 0.2 μg |
| GLA-SE | 5 μg GLA/2% | L7F_A1_23-His | 0.2 μg |
| | squalene | sF_A1_MFur-His | 0.2 μg |
| GLA-3M-052-LS | 10 μg GLA/4 μg | L7F_A1_23-His | 0.2 μg |
| | 3M-052 | sF_A1_MFur-His | 0.2 μg |
| Addavax | 0.25% sorbitol | L7F_A1_23-His | 0.2 μg |
| | trioleate/2.5% | sF_A1_MFur-His | 0.2 μg |
| | squalene/0.25% | | |
| | Tween 80 | | |

*antigen dose per one injection

Mice were immunized with three doses of the compositions as shown in Table 5. Afterword, mice were challenged with hMPV strain, genotype subgroup A1. Sera were taken and used in the MNA assay for assessment of neutralizing antibody titers.

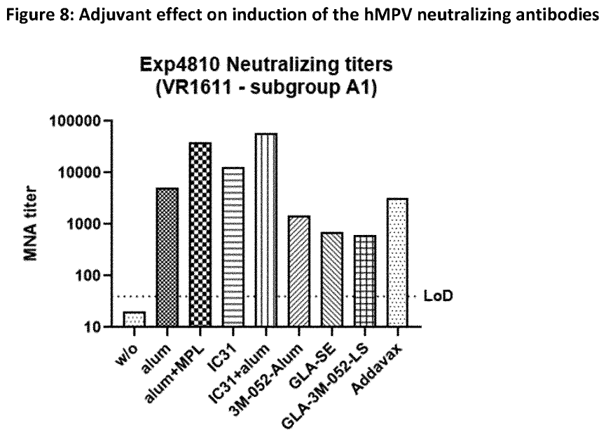

As the result, all tested adjuvants demonstrated enhancement of production of neutralizing antibodies against the homologous hMPV in mice. At the same time, no neutralizing antibodies could be detected in the absence of adjuvants. The combination of pre- and post-fusion F proteins formulated with the adjuvants alum+MPL and IC31_{high}+alum generated the highest amount of neutralizing antibodies. A bit weaker effect was observed for the compositions with 3M-052-Alum and Addavax, The results are shown in FIG. 8.

From these experiments none of the tested adjuvant can be excluded from further testing in other animal species and humans.

SEQUENCES

SEQ ID NO: 11
L7F_A1_23 protein sequence with purification tags
MSWKVVIIFSLLITPQHGLKESYLEESCSTITEGYLSVLRTGWYTNVFTLEVGDVENLTCADGPSLIK
TELDLTKSALRELRTVSADQLAREEQIEQPRQSGCGAGATAGVAIAKTIRLESEVTAIKNALKKTNEA
VSTLGNGVRVLATAVRELKDFVSKNLTRAINKNKCDIADLKMAVSFSQFNRRFLNVVRQFSDNAGITP
AISLDLMTDAELARAVSNMPTSAGQIKLMLENRAMVRRKGFGFLIGVYGSSVIYMVQLPIFGVIDTPC
WIVKAAPSCSEKKGNYACLLREDQGWYCQNAGSTVYYPNEKDCETRGDHVFCDTCAGINVAEQSKECN
INISTTNYPCKVSTGRHPISMVALSPLGALVACYKGVSCSIGSNRVGIIKQLNKGCSYITNQDADTVT
IDNTVYQLSKVEGEQHVIKGRPVSSSFDPVKFPEDQFNVALDQVFESIENSQALVDQSNRILSAGYIP
EAPRDGQAYVRKDGEWVLLSTFLGGLVPRGSHHHHHHSAWSHPQFEK
L7F_A1_23 mature protein sequence
MSWKVVIIFSLLITPQHGLKESYLEESCSTITEGYLSVLRTGWYTNVFTLEVGDVENLTCADGPSLIK
TELDLTKSALRELRTVSADQLAREEQIEQPRQSGCGAGATAGVAIAKTIRLESEVTAIKNALKKTNEA
VSTLGNGVRVLATAVRELKEFVSKNLTRAINKNKCDIADLKMAVSFSQFNRRFLNVVRQFSDNAGITP
AISLDLMTDAELARAVSNMPTSAGQIKLMLENRAMVRRKGFGFLIGVYGSSVIYMVQLPIFGVIDTPC
WIVKAAPSCSEKKGNYACLLREDQGWYCQNAGSTVYYPNEKDCETRGDHVFCDTCAGINVAEQSKECN
INISTTNYPCKVSTGRHPISMVALSPLGALVACYKGVSCSIGSNRVGIIKQLNKGCSYITNQDADTVT
IDNTVYQLSKVEGEQHVIKGRPVSSSFDPVKFPEDQFNVALDQVFESIENSQALVDQSNRILSAGYIP
EAPRDGQAYVRKDGEWVLLSTFL
SEQ ID NO: 12
L7F_B1_23 protein sequence with purification tags
MSWKVMIIISLLITPQHGLKESYLEESCSTITEGYLSVLRTGWYTNVFTLEVGDVENLTCTDGPSLIK
TELDLTKSALRELKTVSADQLAREEQIEQPRQSGCGAGATAGIAIAKTIRLESEVNAIKGALKQTNEA
VSTLGNGVRVLATAVRELKEFVSKNLTSAINRNKCDIADLKMAVSFSQFNRRFLNVVRQFSDNAGITP
AISLDLMTDAELARAVSYMPTSAGQIKLMLENRAMVRRKGFGILIGVYGSSVIYMVQLPIFGVIDTPC
WIIKAAPSCSEKNGNYACLLREDQGWYCKNAGSTVYYPNEKDCETRGDHVFCDTCAGINVAEQSRECN
INISTTNYPCKVSTGRHPISMVALSPLGALVACYKGVSCSIGSNWVGIIKQLPKGCSYITNQDADTVT
IDNTVYQLSKVEGEQHVIKGRPVSSSFDPIKFPEDQFNVALDQVFESIENSQALVDQSNKILNAGYIP
EAPRDGQAYVRKDGEWVLLSTFLGGLVPRGSHHHHHHSAWSHPQFEK
L7F_B1_23 mature protein sequence
MSWKVMIIISLLITPQHGLKESYLEESCSTITEGYLSVLRTGWYTNVFTLEVGDVENLTCTDGPSLIK
TELDLTKSALRELKTVSADQLAREEQIEQPRQSGCGAGATAGIAIAKTIRLESEVNAIKGALKQTNEA
VSTLGNGVRVLATAVRELKEFVSKNLTSAINRNKCDIADLKMAVSFSQFNRRFLNVVRQFSDNAGITP
AISLDLMTDAELARAVSYMPTSAGQIKLMLENRAMVRRKGFGILIGVYGSSVIYMVQLPIFGVIDTPC
WIIKAAPSCSEKNGNYACLLREDQGWYCKNAGSTVYYPNEKDCETRGDHVFCDTCAGINVAEQSRECN
INISTTNYPCKVSTGRHPISMVALSPLGALVACYKGVSCSIGSNWVGIIKQLPKGCSYITNQDADTVT
IDNTVYQLSKVEGEQHVIKGRPVSSSFDPIKFPEDQFNVALDQVFESIENSQALVDQSNKILNAGYIP
EAPRDGQAYVRKDGEWVLLSTFL
SEQ ID NO: 13
L7F_A1_23.2 protein sequence with purification tags
MSWKVVIIFSLLITPQHGLKESYLEESCSTITEGYLSVLRTGWYTNVFTLEVGDVENLTCADGPSLIK
TELDLTKSALRELRTVSADQLAREEQIEQPRQSGCGAGVTAGVAIAKTIRLESEVTAIKNALKKTNEA
VSTLGNGVRVLATAVRELKDFVSKNLTRAINKNKCDIADLKMAVSFSQFNRRFLNVVRQFSDNAGITP
AISLDLMTDAELARAVSNMPTSAGQIKLMLENRAMVRRKGFGFLIGVYGSSVIYMVQLPIFGVIDTPC
WIVKAAPSCSEKKGNYACLLREDQGWYCQNAGSTVYYPNEKDCETRGDHVFCDTCAGINVAEQSKECN
INISTTNYPCKVSTGRHPISMVALSPLGALVACYKGVSCSIGSNRVGIIKQLNKGCSYITNQDADTVT
IDNTVYQLSKVEGEQHVIKGRPVSSSFDPVKFPEDQFNVALDQVFESIENSQALVDQSNRILSAGYIP
EAPRDGQAYVRKDGEWVLLSTFLGGLVPRGSHHHHHHSAWSHPQFEK
L7F_A1_23.2 mature protein sequence
MSWKVVIIFSLLITPQHGLKESYLEESCSTITEGYLSVLRTGWYTNVFTLEVGDVENLTCADGPSLIK
TELDLTKSALRELRTVSADQLAREEQIEQPRQSGCGAGVTAGVAIAKTIRLESEVTAIKNALKKTNEA
VSTLGNGVRVLATAVRELKDFVSKNLTRAINKNKCDIADLKMAVSFSQFNRRFLNVVRQFSDNAGITP
AISLDLMTDAELARAVSNMPTSAGQIKLMLENRAMVRRKGFGFLIGVYGSSVIYMVQLPIFGVIDTPC
WIVKAAPSCSEKKGNYACLLREDQGWYCQNAGSTVYYPNEKDCETRGDHVFCDTCAGINVAEQSKECN
INISTTNYPCKVSTGRHPISMVALSPLGALVACYKGVSCSIGSNRVGIIKQLNKGCSYITNQDADTVT
IDNTVYQLSKVEGEQHVIKGRPVSSSFDPVKFPEDQFNVALDQVFESIENSQALVDQSNRILSAGYIP
EAPRDGQAYVRKDGEWVLLSTEL
SEQ ID NO: 14
L7F_B1_23.2 protein sequence with purification tags
MSWKVMIIISLLITPQHGLKESYLEESCSTITEGYLSVLRTGWYTNVFTLEVGDVENLTCTDGPSLIK
TELDLTKSALRELKTVSADQLAREEQIEQPRQSGCGAGVTAGIAIAKTIRLESEVNAIKGALKQTNEA
VSTLGNGVRVLATAVRELKEFVSKNLTSAINRNKCDIADLKMAVSFSQFNRRFLNVVRQFSDNAGITP
AISLDLMTDAELARAVSYMPTSAGQIKLMLENRAMVRRKGFGILIGVYGSSVIYMVQLPIFGVIDTPC
WIIKAAPSCSEKNGNYACLLREDQGWYCKNAGSTVYYPNEKDCETRGDHVFCDTCAGINVAEQSRECN
INISTTNYPCKVSTGRHPISMVALSPLGALVACYKGVSCSIGSNWVGIIKQLPKGCSYITNQDADTVT
IDNTVYQLSKVEGEQHVIKGRPVSSSFDPIKFPEDQFNVALDQVFESIENSQALVDQSNKILNAGYIP
EAPRDGQAYVRKDGEWVLLSTFLGGLVPRGSHHHHHHSAWSHPQFEK*
L7F_B1_23.2 mature protein sequence
MSWKVMIIISLLITPQHGLKESYLEESCSTITEGYLSVLRTGWYTNVFTLEVGDVENLTCTDGPSLIK
TELDLTKSALRELKTVSADQLAREEQIEQPRQSGCGAGVTAGIAIAKTIRLESEVNAIKGALKQTNEA
VSTLGNGVRVLATAVRELKEFVSKNLTSAINRNKCDIADLKMAVSFSQFNRRFLNVVRQFSDNAGITP
AISLDLMTDAELARAVSYMPTSAGQIKLMLENRAMVRRKGFGILIGVYGSSVIYMVQLPIFGVIDTPC
WIIKAAPSCSEKNGNYACLLREDQGWYCKNAGSTVYYPNEKDCETRGDHVFCDTCAGINVAEQSRECN
INISTTNYPCKVSTGRHPISMVALSPLGALVACYKGVSCSIGSNWVGIIKQLPKGCSYITNQDADTVT
IDNTVYQLSKVEGEQHVIKGRPVSSSFDPIKFPEDQFNVALDQVFESIENSQALVDQSNKILNAGYIP
EAPRDGQAYVRKDGEWVLLSTFL
SEQ ID NO: 15
sF_A1_K_L7 protein sequence with purification tags
MSWKVVIIFSLLITPQHGLKESYLEESCSTITEGYLSVLRTGWYTNVFTLEVGDVENLTCADGPSLIK
TELDLTKSALRELRTVSADQLAREEQIEQPRQSGCGAGATAGVAIAKTIRLESEVTAIKNALKKTNEA
VSTLGNGVRVLAFAVRELKDFVSKNLTRALNKNKCDIADLKMAVSFSQFNRRFLNVVRQFSDNAGITP
AISLDLMTDAELARAVSNMPTSAGQIKLMLENRAMVRRKGFGFLIGVYGSSVIYMVQLPIFGVIDTPC
WIVKAAPSCSEKKGNYACLLREDQGWYCQNAGSTVYYPNEKDCETRGDHVFCDTCAGINVAEQSKECN
INISTTNYPCKVSTGRHPISMVALSPLGALVACYKGVSCSIGSNRVGIIKQLNKGCSYITNQDADTVT
IDNTVYQLSKVEGEQHVIKGRPVSSSFDPVKFPEDQFNVALDQVFESIENSQALVDQSNRILSSAESA
IGGYIPEAPRDGQAYVRKDGEWVLLSTFLGGLVPRGSHHHHHHSAWSHPQFEK
sF_A1_K L7 mature protein sequence
LKESYLEESCSTITEGYLSVLRTGWYTNVFTLEVGDVENLTCADGPSLIKTELDLTKSALRELRTVSA
DQLAREEQIEQPRQSGCGAGATAGVAIAKTIRLESEVTAIKNALKKTNEAVSTLGNGVRVLAFAVREL
KDFVSKNLTRALNKNKCDIADLKMAVSFSQFNRRFLNVVRQFSDNAGITPAISLDLMTDAELARAVSN
MPTSAGQIKLMLENRAMVRRKGFGFLIGVYGSSVIYMVQLPIFGVIDTPCWIVKAAPSCSEKKGNYAC
LLREDQGWYCQNAGSTVYYPNEKDCETRGDHVFCDTCAGINVAEQSKECNINISTTNYPCKVSTGRHP
ISMVALSPLGALVACYKGVSCSIGSNRVGIIKQLNKGCSYITNQDADTVTIDNTVYQLSKVEGEQHVI
KGRPVSSSFDPVKFPEDQFNVALDQVFESIENSQALVDQSNRILSSAESAIGGYIPEAPRDGQAYVRK
DGEWVLLSTFL
SEQ ID NO: 16
L7F_A1_31 protein sequence with purification tags
MSWKVVIIFSLLITPQHGLKESYLEESCSTITEGYLSVLRTGWYTNVFMLEVGDVENLTCADGPSLLK
TELDLTKSALRNLRTVSADQLAREEQIEQPRQSGCGAGATAGVAIAKTIRLESEVTAIKNALKKTNEA
VSTLGNGVRVLATMVRELKDFVSKNLTRAINKNKCDIADLKMAVSFSQFNRRFLNVVRQFSDNAGITP
AISLDLMTDAELARAVSNMPTSAGQIKLMLENRAMVRRKGFGILIGVYGSSVIYMVQLPIFGVIDTPC
WIVKAAPSCSEKKGNYACLLREDQGWYCQNAGSTVYYPNEKDCETRGDHVFCDTCAGINVAEQSKECN
INISTTNYPCKVSTGRHPISMVALSPLGALVACYKGVSCSIGSNRVGIIKQLNKGCSYITNQDADTVT
IDNTVYQLSKVEGEQHVIKGRPVSSSFDPVKFPEDQFNVALDQVFESIENSQALVDQSNRILSAGYIP
EAPRDGQAYVRKDGEWVLLSTFLGGLVPRGSHHHHHHSAWSHPQFEK
L7F_A1_31 mature protein sequence without purification tags
LKESYLEESCSTITEGYLSVLRTGWYTNVFMLEVGDVENLTCADGPSLLKTELDLTKSALRNLRTVSA
DQLAREEQIEQPRQSGCGAGATAGVAIAKTIRLESEVTAIKNALKKTNEAVSTLGNGVRVLATMVREL
KDFVSKNLTRAINKNKCDIADLKMAVSFSQFNRRFLNVVRQFSDNAGITPAISLDLMTDAELARAVSN
MPTSAGQIKLMLENRAMVRRKGFGILIGVYGSSVIYMVQLPIFGVIDTPCWIVKAAPSCSEKKGNYAC
LLREDQGWYCQNAGSTVYYPNEKDCETRGDHVFCDTCAGINVAEQSKECNINISTTNYPCKVSTGRHP
ISMVALSPLGALVACYKGVSCSIGSNRVGIIKQLNKGCSYITNQDADTVTIDNTVYQLSKVEGEQHVI
KGRPVSSSFDPVKFPEDQFNVALDQVFESIENSQALVDQSNRILSAGYIPEAPRDGQAYVRKDGEWVL
LSTFL
SEQ ID NO: 17
L7F_A1_33 protein sequence with purification tags
MSWKVVIIFSLLITPQHGLKESYLEESCSTITEGYLSVLRTGWYTNVFMLCVGDVENLTCADGPSLLK
TELDLTKSALRELRTVSADQLAREEQIEQPRQSGCGAGATAGVAIAKTIRLESEVTAIKNALKKTNEA
VSTLGNGVRVLATMVRELCDFVSKNLTRAINKNKCDIADLKMAVSFSQFNRRFLNVVRQFSDNAGITP
AISLDLMTDAELARAVSNMPTSAGQIKLMLENRAMVRRKGFGFLIGVYGSDVIYMVQLPIFGVIDTPC
WIVKAAPSCSEKKGNYACLLREDQGWYCQNAGSTVYYPNEKDCETRGDHVFCDTCAGINVAEQSKECN
INISTTNYPCKVSTGRHPISMVALSPLGALVACYKGVSCSIGSNRVGIIKQLNKGCSYITNQDADTVT
IDNTVYQLSKVEGEQHVIKGRPVSSSFDPVKFPEDQFNVALDQVFESIENSQALVDQSNRCCSAGYIP
EAPRDGQAYVRKDGEWVLLSTFLGGLVPRGSHHHHHHSAWSHPQFEK
L7F_A1_33 mature protein sequence without purification tags
LKESYLEESCSTITEGYLSVLRTGWYTNVFMLCVGDVENLTCADGPSLLKTELDLTKSALRELRTVSA
DQLAREEQIEQPRQSGCGAGATAGVAIAKTIRLESEVTAIKNALKKTNEAVSTLGNGVRVLATMVREL
CDFVSKNLTRAINKNKCDIADLKMAVSFSQFNRRFLNVVRQFSDNAGITPAISLDLMTDAELARAVSN
MPTSAGQIKLMLENRAMVRRKGFGFLIGVYGSDVIYMVQLPIFGVIDTPCWIVKAAPSCSEKKGNYAC
LLREDQGWYCQNAGSTVYYPNEKDCETRGDHVFCDTCAGINVAEQSKECNINISTTNYPCKVSTGRHP
ISMVALSPLGALVACYKGVSCSIGSNRVGIIKQLNKGCSYITNQDADTVTIDNTVYQLSKVEGEQHVI
KGRPVSSSFDPVKFPEDQFNVALDQVFESIENSQALVDQSNRCCSAGYIPEAPRDGQAYVRKDGEWVL
LSTFL
SEQ ID NO: 18
L7F_A1_4.2 protein sequence with purification tags
MSWKVVIIFSLLITPQHGLKESYLEESCSTITEGYLSVLRTGWYTNVEMLEVGDVENLTCADGPSLIK
TELDLTKSALRELRTVSADQLAREEQIEQPRQSGCGAGATAGVAIAKTIRLESEVTAWKNALKKTNEV
VSTLGNGVRVLVTMVRELKDFVSKNLTRALNKNKCDIADLKMAVSFSQFNRRFLNVVRQFSDNAGITP
AISLDLMTDAELARAVSNMPTSAGQIKLMLENRAMVRRKGFGFLIGVYGSSVIYMVQLPIFGVIDTPC
WIVKAAPSCSEKKGNYACLLREDQGWYCQNAGSTVYYPNEKDCETRGDHVFCDTCAGINVAEQSKECN
INISTTNYPCKVSTGRHPISMVALSPLGALVACYKGVSCSIGSNRVGIIKQLNKGCSYITNQDADTVT
IDNTVYQLSKVEGEQHVIKGRPVSSSFDPVKFPEDQFNVALDQVFESIENSQALVDQSNRILSSAESA
IGGYIPEAPRDGQAYVRKDGEWVLLSTFLGGLVPRGSHHHHHHSAWSHPQFEK
L7F_A1_4.2 mature protein sequence without purification tags
LKESYLEESCSTITEGYLSVLRTGWYTNVEMLEVGDVENLTCADGPSLIKTELDLTKSALRELRTVSA
DQLAREEQIEQPRQSGCGAGATAGVAIAKTIRLESEVTAWKNALKKTNEVVSTLGNGVRVLVTMVREL
KDFVSKNLTRALNKNKCDIADLKMAVSFSQFNRRFLNVVRQFSDNAGITPAISLDLMTDAELARAVSN
MPTSAGQIKLMLENRAMVRRKGFGFLIGVYGSSVIYMVQLPIFGVIDTPCWIVKAAPSCSEKKGNYAC
LLREDQGWYCQNAGSTVYYPNEKDCETRGDHVFCDTCAGINVAEQSKECNINISTTNYPCKVSTGRHP
ISMVALSPLGALVACYKGVSCSIGSNRVGIIKQLNKGCSYITNQDADTVTIDNTVYQLSKVEGEQHVI
KGRPVSSSFDPVKFPEDQFNVALDQVFESIENSQALVDQSNRILSSAESAIGGYIPEAPRDGQAYVRK
DGEWVLLSTFL
SEQ ID NO: 19
sF_A1_K-E294 two polypeptide chain protein sequence with trimerization helper KLL and
purification tags
MSWKVVIIFSLLITPQHGLKESYLEESCSTITEGYLSVLRTGWYTNVFTLEVGDVENLTCADGPSLIK
TELDLTKSALRELRTVSADQLAREEQIENPRQSRFVLGAIALGVCTAAAVTAGVAIAKTIRLESEVTA
IKNALKKTNEAVSTLGNGVRVLAFAVRELKDFVSKNLTRALNKNKCDIADLKMAVSFSQFNRRFLNVV
RQFSDNAGITPAISLDLMTDAELARAVSNMPTSAGQIKLMLENRAMVRRKGFGFLIGVYGSSVIYMVQ
LPIFGVIDTPCWIVKAAPSCSEKKGNYACLLREDQGWYCQNAGSTVYYPNEKDCETRGDHVFCDTACG
INVAEQSKECNINISTTNYPCKVSTGRHPISMVALSPLGALVACYKGVSCSIGSNRVGIIKQLNKGCS
YITNQDADTVTIDNTVYQLSKVEGEQHVIKGRPVSSSFDPVKFPEDQFNVALDQVFESIENSQALVDQ
SNRILSSAESAIGGYIPEAPRDGQAYVRKDGEWVLLSTFLGGLVPRGSHHHHHHSAWSHPQFEK
SEQ ID NO: 20
sF_B1_K-E294 two polypeptide chain protein sequence with trimerization helper KLL and
purification tags
MSWKVMIIISLLITPQHGLKESYLEESCSTITEGYLSVLRTGWYTNVFTLEVGDVENLTCTDGPSLIK
TELDLTKSALRELKTVSADQLAREEQIENPRQSRFVLGAIALGVCTAAAVTAGIAIAKTIRLESEVNA
IKGALKQTNEAVSTLGNGVRVLAFAVRELKEFVSKNLTSALNRNKCDIADLKMAVSFSQFNRRFLNVV
RQFSDNAGITPAISLDLMTDAELARAVSYMPTSAGQIKLMLENRAMVRRKGFGILIGVYGSSVIYMVQ
LPIFGVIDTPCWIIKAAPSCSEKNGNYACLLREDQGWYCKNAGSTVYYPNEKDCETRGDHVFCDTACG
INVAEQSRECNINISTTNYPCKVSTGRHPISMVALSPLGALVACYKGVSCSIGSNWVGIIKQLPKGCS
YITNQDADTVTIDNTVYQLSKVEGEQHVIKGRPVSSSFDPIKFPEDQFNVALDQVFESIENSQALVDQ
SNKILNSAESAIGGYIPEAPRDGQAYVRKDGEWVLLSTFLGGLVPRGSHHHHHHSAWSHPQFEK
SF_B1_K-E294 without without purification tags
MSWKVMIIISLLITPQHGLKESYLEESCSTITEGYLSVLRTGWYTNVFTLEVGDVENLTCTDGPSLIK
TELDLTKSALRELKTVSADQLAREEQIENPRQSRFVLGAIALGVCTAAAVTAGIAIAKTIRLESEVNA
IKGALKQTNEAVSTLGNGVRVLAFAVRELKEFVSKNLTSALNRNKCDIADLKMAVSESQFNRRFLNVV
RQFSDNAGITPAISLDLMTDAELARAVSYMPTSAGQIKLMLENRAMVRRKGFGILIGVYGSSVIYMVQ
LPIFGVIDTPCWIIKAAPSCSEKNGNYACLLREDQGWYCKNAGSTVYYPNEKDCETRGDHVFCDTACG
INVAEQSRECNINISTTNYPCKVSTGRHPISMVALSPLGALVACYKGVSCSIGSNWVGIIKQLPKGCS
YITNQDADTVTIDNTVYQLSKVEGEQHVIKGRPVSSSFDPIKFPEDQFNVALDQVFESIENSQALVDQ
SNKILNSAESAIGGYIPEAPRDGQAYVRKDGEWVLLSTFL
SEQ ID NO: 21
sF_A1_MFur protein sequence with purification tags stabilized in the
post-fusion conformation
MSWKVVIIFSLLITPQHGLKESYLEESCSTITEGYLSVLRTGWYTNVFTLEVGDVENLTCADGPSLIK
TELDLTKSALRELRTVSADQLAREEQIENPRQSKKRKRRVATAAAVTAGVAIAKTIRLESEVTAIKNA
LKKTNEAVSTLGNGVRVLATAVRELKDFVSKNLTRAINKNKCDIADLKMAVSESQFNRRFLNVVRQFS
DNAGITPAISLDLMTDAELARAVSNMPTSAGQIKLMLENRAMVRRKGFGFLIGVYGSSVIYMVQLPIF
GVIDTPCWIVKAAPSCSEKKGNYACLLREDQGWYCQNAGSTVYYPNEKDCETRGDHVFCDTAAGINVA
EQSKECNINISTTNYPCKVSTGRHPISMVALSPLGALVACYKGVSCSIGSNRVGIIKQLNKGCSYITN
QDADTVTIDNTVYQLSKVEGEQHVIKGRPVSSSFDPVKFPEDQFNVALDQVFESIENSQALVDQSNRI
LSSAEKGNTSGRENLYFQGGGGSGYIPEAPRDGQAYVRKDGEWVLLSTFLGGIEGRHHHHHH
sF_A1_MFur without without purification tags
MSWKVVIIFSLLITPQHGLKESYLEESCSTITEGYLSVLRTGWYTNVFTLEVGDVENLTCADGPSLIK
TELDLTKSALRELRTVSADQLAREEQIENPRQSKKRKRRVATAAAVTAGVAIAKTIRLESEVTAIKNA
LKKTNEAVSTLGNGVRVLATAVRELKDFVSKNLTRAINKNKCDIADLKMAVSFSQFNRRFLNVVRQFS
DNAGITPAISLDLMTDAELARAVSNMPTSAGQIKLMLENRAMVRRKGFGFLIGVYGSSVIYMVQLPIF
GVIDTPCWIVKAAPSCSEKKGNYACLLREDQGWYCQNAGSTVYYPNEKDCETRGDHVFCDTAAGINVA
EQSKECNINISTTNYPCKVSTGRHPISMVALSPLGALVACYKGVSCSIGSNRVGIIKQLNKGCSYITN
QDADTVTIDNTVYQLSKVEGEQHVIKGRPVSSSFDPVKFPEDQFNVALDQVFESIENSQALVDQSNRI
LSSAEKGNTSGRENLYFQGGGGSGYIPEAPRDGQAYVRKDGEWVLLSTFL
SEQ ID NO: 22
sF_B1_MFur protein sequence with purification tags, stabilized in the
post-fusion conformation
MSWKVMIIISLLITPQHGLKESYLEESCSTITEGYLSVLRTGWYTNVFTLEVGDVENLTCTDGPSLIK
TELDLTKSALRELKTVSADQLAREEQIENPRQSKKRKRRVATAAAVTAGIAIAKTIRLESEVNAIKGA
LKQTNEAVSTLGNGVRVLATAVRELKEFVSKNLTSAINRNKCDIADLKMAVSFSQFNRRFLNVVRQFS
DNAGITPAISLDLMTDAELARAVSYMPTSAGQIKLMLENRAMVRRKGFGILIGVYGSSVIYMVQLPIF
GVIDTPCWIIKAAPSCSEKNGNYACLLREDQGWYCKNAGSTVYYPNEKDCETRGDHVFCDTAAGINVA
EQSRECNINISTTNYPCKVSTGRHPISMVALSPLGALVACYKGVSCSIGSNWVGIIKQLPKGCSYITN
QDADTVTIDNTVYQLSKVEGEQHVIKGRPVSSSFDPIKFPEDQFNVALDQVFESIENSQALVDQSNKI
LNSAEKGNTSGRENLYFQGGGGSGYIPEAPRDGQAYVRKDGEWVLLSTFLGGLVPRGSHHHHHHSAWS
HPQFEK
SF_B1_MFur without without purification tags
MSWKVMIIISLLITPQHGLKESYLEESCSTITEGYLSVLRTGWYTNVFTLEVGDVENLTCTDGPSLIK
TELDLTKSALRELKTVSADQLAREEQIENPRQSKKRKRRVATAAAVTAGIAIAKTIRLESEVNAIKGA
LKQTNEAVSTLGNGVRVLATAVRELKEFVSKNLTSAINRNKCDIADLKMAVSFSQFNRRFLNVVRQFS
DNAGITPAISLDLMTDAELARAVSYMPTSAGQIKLMLENRAMVRRKGFGILIGVYGSSVIYMVQLPIF
GVIDTPCWIIKAAPSCSEKNGNYACLLREDQGWYCKNAGSTVYYPNEKDCETRGDHVFCDTAAGINVA
EQSRECNINISTTNYPCKVSTGRHPISMVALSPLGALVACYKGVSCSIGSNWVGIIKQLPKGCSYITN
QDADTVTIDNTVYQLSKVEGEQHVIKGRPVSSSFDPIKFPEDQFNVALDQVFESIENSQALVDQSNKI
LNSAEKGNTSGRENLYFQGGGGSGYIPEAPRDGQAYVRKDGEWVLLSTFL
SEQ ID NO: 23
Trimerization helper domain (foldon) from fibritin of T4 bacteriophage
GYIPEAPRDGQAYVRKDGEWVLLSTFL
SEQ ID NO: 24
Foldon-glyc-1
GYIPEAPRNGTAYVRKDGEWVLLSTFL
SEQ ID NO: 25
Foldon-glyc-2
GYIPEAPRDGQAYVRKNGTWVLLSTFL
SEQ ID NO: 26
Foldon-glyc-3
GYIPEAPRDGQAYVRKDGNWTLLSTFL
SEQ ID NO: 27
Foldon-glyc-4
GYIPEAPRNGTAYVRKNGTWVLLSTFL
SEQ ID NO: 28
Foldon-glyc-5
GYIPEAPRNGTAYVRKDGNWTLLSTFL
SEQ ID NO: 29
Trimerization helper VSLmotif
ILSA
SEQ ID NO: 30
Trimerization helper VSAmotif
CCSA
SEQ ID NO: 31
L7F_A1_23 coding nucleotide sequence, codon optimized
ATGTCTTGGAAGGTGGTCATCATCTTCTCCCTGCTGATCACCCCTCAGCACGGCCTGAAAGAGTCCTA
CCTGGAAGAGAGCTGCTCCACCATCACCGAGGGCTACCTGTCTGTGCTGAGAACCGGCTGGTACACCA
ACGTGTTCACCCTGGAAGTGGGCGACGTGGAAAACCTGACCTGTGCTGATGGCCCCAGCCTGATCAAG
ACCGAGCTGGACCTGACCAAGTCTGCCCTGAGAGAACTGAGGACCGTGTCTGCCGATCAGCTGGCCAG
AGAGGAACAGATCGAGCAGCCTAGACAGTCCGGATGTGGTGCTGGTGCTACAGCTGGCGTGGCCATTG
CCAAGACCATCCGGCTGGAATCTGAAGTGACCGCCATCAAGAACGCCCTGAAAAAGACCAACGAGGCC
GTGTCTACCCTCGGCAATGGCGTTAGAGTGCTGGCCACAGCCGTGCGCGAGCTGAAGGATTTCGTGTC
CAAGAACCTGACCAGGGCCATCAACAAGAACAAGTGTGATATCGCCGACCTGAAGATGGCCGTGTCCT
TCAGCCAGTTCAACCGGCGGTTCCTGAATGTCGTGCGGCAGTTCTCTGACAACGCCGGCATCACCCCT
GCCATCAGCCTGGATCTGATGACCGATGCCGAGCTGGCTAGAGCCGTGTCCAACATGCCTACCTCTGC
CGGCCAGATCAAGCTGATGCTGGAAAACAGAGCCATGGTCCGACGGAAAGGCTTCGGCTTTCTGATCG
GCGTGTACGGCTCCTCCGTGATCTACATGGTGCAGCTGCCTATCTTCGGCGTGATCGACACCCCTTGC
TGGATCGTGAAGGCCGCTCCTAGCTGCTCTGAGAAGAAGGGCAACTACGCCTGCCTGCTGAGAGAGGA
CCAAGGCTGGTACTGTCAGAACGCCGGCTCCACCGTGTACTACCCCAACGAGAAGGACTGCGAGACAA
GAGGCGACCACGTGTTCTGCGATACCTGCGCTGGCATCAATGTGGCCGAGCAGTCCAAAGAGTGCAAC
ATCAACATCTCCACCACCAACTATCCCTGCAAGGTGTCCACCGGCAGGCACCCTATTTCCATGGTGGC
TCTGTCTCCACTGGGCGCCCTGGTGGCTTGTTATAAGGGCGTGTCCTGCTCCATCGGCTCCAACAGAG
TGGGCATCATCAAGCAGCTGAACAAGGGCTGCAGCTACATCACCAACCAGGACGCCGATACCGTGACC
ATCGACAATACCGTGTATCAGCTGTCCAAGGTGGAAGGCGAGCAGCACGTGATCAAGGGCAGACCTGT
GTCCTCCAGCTTCGACCCCGTGAAGTTCCCTGAGGATCAGTTCAACGTGGCCCTGGACCAGGTGTTCG
AGTCCATCGAGAACTCTCAGGCTCTGGTGGACCAGTCCAACCGGATTCTGTCTGCCGGCTACATCCCC
GAGGCTCCTAGAGATGGACAGGCCTACGTCAGAAAGGACGGCGAATGGGTGCTGCTGTCTACCTTTCT
CGGAGGCCTGGTGCCTAGAGGCTCTCACCACCATCATCACCACTCCGCTTGGTCCCATCCACAGTTCG
AGAAGTGA
SEQ ID NO: 32
L7F_B1_23 coding nucleotide sequence, codon optimized
ATGTCTTGGAAAGTTATGATTATTATTTCTTTGTTGATTACTCCACAACATGGTTTGAAAGAATCTTA
TTTGGAAGAATCTTGTTCTACTATTACTGAAGGTTATTTGTCTGTTTTGAGAACTGGTTGGTATACTA
ATGTTTTTACTTTGGAAGTTGGTGATGTTGAAAATTTGACTTGTACTGATGGTCCATCTTTGATTAAA
ACTGAATTGGATTTGACTAAATCTGCTTTGAGAGAATTGAAAACTGTTTCTGCTGATCAATTGGCTAG
AGAAGAACAAATTGAACAACCAAGACAATCTGGTTGTGGTGCTGGTGCTACTGCTGGTATTGCTATTG
CTAAAACTATTAGATTGGAATCTGAAGTTAATGCTATTAAAGGTGCTTTGAAACAAACTAATGAAGCT
GTTTCTACTTTGGGTAATGGTGTTAGAGTTTTGGCTACTGCTGTTAGAGAATTGAAAGAATTTGTTTC
TAAAAATTTGACTTCTGCTATTAATAGAAATAAATGTGATATTGCTGATTTGAAAATGGCTGTTTCTT
TTTCTCAATTTAATAGAAGATTTTTGAATGTTGTTAGACAATTTTCTGATAATGCTGGTATTACTCCA
GCTATTTCTTTGGATTTGATGACTGATGCTGAATTGGCTAGAGCTGTTTCTTATATGCCAACTTCTGC
TGGTCAAATTAAATTGATGTTGGAAAATAGAGCTATGGTTAGAAGAAAAGGTTTTGGTATTTTGATTG
GTGTTTATGGTTCTTCTGTTATTTATATGGTTCAATTGCCAATTTTTGGTGTTATTGATACTCCATGT
TGGATTATTAAAGCTGCTCCATCTTGTTCTGAAAAAAATGGTAATTATGCTTGTTTGTTGAGAGAAGA
TCAAGGTTGGTATTGTAAAAATGCTGGTTCTACTGTTTATTATCCAAATGAAAAAGATTGTGAAACTA
GAGGTGATCATGTTTTTTGTGATACTTGTGCTGGTATTAATGTTGCTGAACAATCTAGAGAATGTAAT
ATTAATATTTCTACTACTAATTATCCATGTAAAGTTTCTACTGGTAGACATCCAATTTCTATGGTTGC
TTTGTCTCCATTGGGTGCTTTGGTTGCTTGTTATAAAGGTGTTTCTTGTTCTATTGGTTCTAATTGGG
TTGGTATTATTAAACAATTGCCAAAAGGTTGTTCTTATATTACTAATCAAGATGCTGATACTGTTACT
ATTGATAATACTGTTTATCAATTGTCTAAAGTTGAAGGTGAACAACATGTTATTAAAGGTAGACCAGT
TTCTTCTTCTTTTGATCCAATTAAATTTCCAGAAGATCAATTTAATGTTGCTTTGGATCAAGTTTTTG
AATCTATTGAAAATTCTCAAGCTTTGGTTGATCAATCTAATAAAATTTTGAATGCTGGTTATATTCCA
GAAGCTCCAAGAGATGGTCAAGCTTATGTTAGAAAAGATGGTGAATGGGTTTTGTTGTCTACTTTTTT
GGGTGGTTTGGTTCCAAGAGGTTCTCATCATCATCATCATCATTCTGCTTGGTCTCATCCACAATTTG
AAAAATGA
SEQ ID NO: 33
L7F_A1_23.2 coding nucleotide sequence, codon optimized
ATGTCTTGGAAAGTTGTTATTATTTTTTCTTTGTTGATTACTCCACAACATGGTTTGAAAGAATCTTA
TTTGGAAGAATCTTGTTCTACTATTACTGAAGGTTATTTGTCTGTTTTGAGAACTGGTTGGTATACTA
ATGTTTTTACTTTGGAAGTTGGTGATGTTGAAAATTTGACTTGTGCTGATGGTCCATCTTTGATTAAA
ACTGAATTGGATTTGACTAAATCTGCTTTGAGAGAATTGAGAACTGTTTCTGCTGATCAATTGGCTAG
AGAAGAACAAATTGAACAACCAAGACAATCTGGTTGTGGTGCTGGTGTTACTGCTGGTGTTGCTATTG
CTAAAACTATTAGATTGGAATCTGAAGTTACTGCTATTAAAAATGCTTTGAAAAAAACTAATGAAGCT
GTTTCTACTTTGGGTAATGGTGTTAGAGTTTTGGCTACTGCTGTTAGAGAATTGAAAGATTTTGTTTC
TAAAAATTTGACTAGAGCTATTAATAAAAATAAATGTGATATTGCTGATTTGAAAATGGCTGTTTCTT
TTTCTCAATTTAATAGAAGATTTTTGAATGTTGTTAGACAATTTTCTGATAATGCTGGTATTACTCCA
GCTATTTCTTTGGATTTGATGACTGATGCTGAATTGGCTAGAGCTGTTTCTAATATGCCAACTTCTGC
TGGTCAAATTAAATTGATGTTGGAAAATAGAGCTATGGTTAGAAGAAAAGGTTTTGGTTTTTTGATTG
GTGTTTATGGTTCTTCTGTTATTTATATGGTTCAATTGCCAATTTTTGGTGTTATTGATACTCCATGT
TGGATTGTTAAAGCTGCTCCATCTTGTTCTGAAAAAAAAGGTAATTATGCTTGTTTGTTGAGAGAAGA
TCAAGGTTGGTATTGTCAAAATGCTGGTTCTACTGTTTATTATCCAAATGAAAAAGATTGTGAAACTA
GAGGTGATCATGTTTTTTGTGATACTTGTGCTGGTATTAATGTTGCTGAACAATCTAAAGAATGTAAT
ATTAATATTTCTACTACTAATTATCCATGTAAAGTTTCTACTGGTAGACATCCAATTTCTATGGTTGC
TTTGTCTCCATTGGGTGCTTTGGTTGCTTGTTATAAAGGTGTTTCTTGTTCTATTGGTTCTAATAGAG
TTGGTATTATTAAACAATTGAATAAAGGTTGTTCTTATATTACTAATCAAGATGCTGATACTGTTACT
ATTGATAATACTGTTTATCAATTGTCTAAAGTTGAAGGTGAACAACATGTTATTAAAGGTAGACCAGT
TTCTTCTTCTTTTGATCCAGTTAAATTTCCAGAAGATCAATTTAATGTTGCTTTGGATCAAGTTTTTG
AATCTATTGAAAATTCTCAAGCTTTGGTTGATCAATCTAATAGAATTTTGTCTGCTGGTTATATTCCA
GAAGCTCCAAGAGATGGTCAAGCTTATGTTAGAAAAGATGGTGAATGGGTTTTGTTGTCTACTTTTTT
GGGTGGTTTGGTTCCAAGAGGTTCTCATCATCATCATCATCATTCTGCTTGGTCTCATCCACAATTTG
AAAAATGA
SEQ ID NO: 34
L7F_B1_23.2 coding nucleotide sequence, codon optimized
ATGTCTTGGAAAGTTATGATTATTATTTCTTTGTTGATTACTCCACAACATGGTTTGAAAGAATCTTA
TTTGGAAGAATCTTGTTCTACTATTACTGAAGGTTATTTGTCTGTTTTGAGAACTGGTTGGTATACTA
ATGTTTTTACTTTGGAAGTTGGTGATGTTGAAAATTTGACTTGTACTGATGGTCCATCTTTGATTAAA
ACTGAATTGGATTTGACTAAATCTGCTTTGAGAGAATTGAAAACTGTTTCTGCTGATCAATTGGCTAG
AGAAGAACAAATTGAACAACCAAGACAATCTGGTTGTGGTGCTGGTGTTACTGCTGGTATTGCTATTG
CTAAAACTATTAGATTGGAATCTGAAGTTAATGCTATTAAAGGTGCTTTGAAACAAACTAATGAAGCT
GTTTCTACTTTGGGTAATGGTGTTAGAGTTTTGGCTACTGCTGTTAGAGAATTGAAAGAATTTGTTTC
TAAAAATTTGACTTCTGCTATTAATAGAAATAAATGTGATATTGCTGATTTGAAAATGGCTGTTTCTT
TTTCTCAATTTAATAGAAGATTTTTGAATGTTGTTAGACAATTTTCTGATAATGCTGGTATTACTCCA
GCTATTTCTTTGGATTTGATGACTGATGCTGAATTGGCTAGAGCTGTTTCTTATATGCCAACTTCTGC
TGGTCAAATTAAATTGATGTTGGAAAATAGAGCTATGGTTAGAAGAAAAGGTTTTGGTATTTTGATTG
GTGTTTATGGTTCTTCTGTTATTTATATGGTTCAATTGCCAATTTTTGGTGTTATTGATACTCCATGT
TGGATTATTAAAGCTGCTCCATCTTGTTCTGAAAAAAATGGTAATTATGCTTGTTTGTTGAGAGAAGA
TCAAGGTTGGTATTGTAAAAATGCTGGTTCTACTGTTTATTATCCAAATGAAAAAGATTGTGAAACTA
GAGGTGATCATGTTTTTTGTGATACTTGTGCTGGTATTAATGTTGCTGAACAATCTAGAGAATGTAAT
ATTAATATTTCTACTACTAATTATCCATGTAAAGTTTCTACTGGTAGACATCCAATTTCTATGGTTGC
TTTGTCTCCATTGGGTGCTTTGGTTGCTTGTTATAAAGGTGTTTCTTGTTCTATTGGTTCTAATTGGG
TTGGTATTATTAAACAATTGCCAAAAGGTTGTTCTTATATTACTAATCAAGATGCTGATACTGTTACT
ATTGATAATACTGTTTATCAATTGTCTAAAGTTGAAGGTGAACAACATGTTATTAAAGGTAGACCAGT
TTCTTCTTCTTTTGATCCAATTAAATTTCCAGAAGATCAATTTAATGTTGCTTTGGATCAAGTTTTTG
AATCTATTGAAAATTCTCAAGCTTTGGTTGATCAATCTAATAAAATTTTGAATGCTGGTTATATTCCA
GAAGCTCCAAGAGATGGTCAAGCTTATGTTAGAAAAGATGGTGAATGGGTTTTGTTGTCTACTTTTTT
GGGTGGTTTGGTTCCAAGAGGTTCTCATCATCATCATCATCATTCTGCTTGGTCTCATCCACAATTTG
AAAAATGA
SEQ ID NO: 35
sF_A1_K_L7 coding nucleotide sequence, codon optimized
ATGTCTTGGAAGGTGGTCATCATCTTCTCCCTGCTGATCACCCCTCAGCACGGCCTGAAAGAGTCCTA
CCTGGAAGAGAGCTGCTCCACCATCACCGAGGGCTACCTGTCTGTGCTGAGAACCGGCTGGTACACCA
ACGTGTTCACCCTGGAAGTGGGCGACGTGGAAAACCTGACCTGTGCTGATGGCCCCAGCCTGATCAAG
ACCGAGCTGGACCTGACCAAGTCTGCCCTGAGAGAACTGAGGACCGTGTCTGCCGATCAGCTGGCCAG
AGAGGAACAGATCGAGCAGCCTAGACAGTCCGGATGTGGTGCTGGTGCTACAGCTGGCGTGGCCATTG
CCAAGACCATCCGGCTGGAATCTGAAGTGACCGCCATCAAGAACGCCCTGAAAAAGACCAACGAGGCC
GTGTCTACCCTCGGCAATGGCGTTAGAGTGCTGGCCTTTGCTGTGCGCGAGCTGAAGGACTTCGTGTC
CAAGAACCTGACCAGGGCTCTGAACAAGAACAAGTGTGATATCGCCGACCTGAAGATGGCCGTGTCCT
TTAGCCAGTTCAACCGGCGGTTCCTGAACGTCGTGCGGCAGTTCTCTGATAACGCCGGCATCACCCCT
GCCATCAGCCTGGATCTGATGACCGATGCCGAGCTGGCTAGAGCCGTGTCCAACATGCCTACCTCTGC
CGGCCAGATCAAGCTGATGCTGGAAAACAGAGCCATGGTCCGACGGAAAGGCTTCGGCTTTCTGATCG
GCGTGTACGGCTCCTCCGTGATCTACATGGTGCAGCTGCCTATCTTCGGCGTGATCGACACCCCTTGC
TGGATCGTGAAGGCCGCTCCTAGCTGCTCTGAGAAGAAGGGCAACTACGCCTGCCTGCTGAGAGAGGA
CCAAGGCTGGTACTGTCAGAACGCCGGCTCCACCGTGTACTACCCCAACGAGAAGGACTGCGAGACAA
GAGGCGACCACGTGTTCTGCGATACCTGCGCTGGCATCAATGTGGCCGAGCAGTCCAAAGAGTGCAAC
ATCAACATCTCCACCACCAACTATCCCTGCAAGGTGTCCACCGGCAGGCACCCTATTTCCATGGTGGC
TCTGTCTCCACTGGGCGCCCTGGTGGCTTGTTATAAGGGCGTGTCCTGCTCCATCGGCTCCAACAGAG
TGGGCATCATCAAGCAGCTGAACAAGGGCTGCAGCTACATCACCAACCAGGACGCCGATACCGTGACC
ATCGACAATACCGTGTATCAGCTGTCCAAGGTGGAAGGCGAGCAGCACGTGATCAAGGGCAGACCTGT
GTCCTCCAGCTTCGACCCCGTGAAGTTCCCTGAGGATCAGTTCAACGTGGCCCTGGACCAGGTGTTCG
AGTCCATCGAGAACTCTCAGGCTCTGGTGGACCAGTCCAACCGGATCCTGTCCTCTGCCGAGTCTGCT
ATCGGCGGCTATATCCCCGAGGCTCCTAGAGATGGCCAGGCCTATGTTCGGAAGGATGGCGAATGGGT
GCTGCTGTCTACCTTCCTCGGAGGCCTGGTGCCTAGAGGCTCTCACCACCATCATCACCACTCCGCTT
GGTCCCATCCACAGTTCGAGAAGTGA
SEQ ID NO: 36
L7F_A1_31 coding nucleotide sequence, codon optimized
ATGTCTTGGAAGGTGGTCATCATCTTCTCCCTGCTGATCACCCCTCAGCACGGCCTGAAAGAGTCCTA
CCTGGAAGAGAGCTGCTCCACCATCACCGAGGGCTACCTGTCTGTGCTGAGAACCGGCTGGTACACCA
ACGTGTTCATGCTGGAAGTGGGCGACGTGGAAAACCTGACCTGTGCTGATGGCCCCAGCCTGCTGAAA
ACAGAGCTGGACCTGACCAAGAGCGCCCTGAGAAATCTGAGGACCGTGTCTGCCGATCAGCTGGCCAG
AGAGGAACAGATCGAGCAGCCTAGACAGTCCGGATGTGGTGCTGGTGCTACAGCTGGCGTGGCCATTG
CCAAGACCATCCGGCTGGAATCTGAAGTGACCGCCATCAAGAATGCCCTGAAAAAGACCAACGAGGCC
GTGTCTACCCTCGGCAATGGCGTTAGAGTGCTGGCCACAATGGTCCGAGAGCTGAAGGACTTCGTGTC
CAAGAACCTGACCAGGGCCATCAACAAGAACAAGTGTGATATCGCCGACCTGAAGATGGCCGTGTCCT
TTAGCCAGTTCAACCGGCGGTTCCTGAACGTCGTGCGGCAGTTCTCTGATAACGCCGGCATCACCCCT
GCCATCAGCCTGGATCTGATGACCGATGCCGAGCTGGCTAGAGCCGTGTCCAACATGCCTACCTCTGC
CGGCCAGATCAAGCTGATGCTCGAGAACAGAGCTATGGTCCGACGGAAAGGCTTCGGCATCCTGATCG
GCGTGTACGGCTCCTCCGTGATCTACATGGTGCAGCTGCCTATCTTCGGCGTGATCGACACCCCTTGC
TGGATCGTGAAGGCCGCTCCTAGCTGCTCTGAGAAGAAGGGCAACTACGCCTGCCTGCTGAGAGAGGA
CCAAGGCTGGTACTGTCAGAACGCCGGCTCCACCGTGTACTACCCCAACGAGAAGGACTGCGAGACAA
GAGGCGACCACGTGTTCTGCGATACCTGCGCTGGCATCAATGTGGCCGAGCAGTCCAAAGAGTGCAAC
ATCAACATCTCCACCACCAACTATCCCTGCAAGGTGTCCACCGGCAGGCACCCTATTTCCATGGTGGC
TCTGTCTCCACTGGGCGCCCTGGTGGCTTGTTATAAGGGCGTGTCCTGCTCCATCGGCTCCAACAGAG
TGGGCATCATCAAGCAGCTGAACAAGGGCTGCAGCTACATCACCAACCAGGACGCCGATACCGTGACC
ATCGACAATACCGTGTATCAGCTGTCCAAGGTGGAAGGCGAGCAGCACGTGATCAAGGGCAGACCTGT
GTCCTCCAGCTTCGACCCCGTGAAGTTCCCTGAGGATCAGTTCAACGTGGCCCTGGACCAGGTGTTCG
AGTCCATCGAGAACTCTCAGGCTCTGGTGGACCAGTCCAACCGGATTCTGTCTGCCGGCTACATCCCC
GAGGCTCCTAGAGATGGACAGGCCTACGTCAGAAAGGACGGCGAATGGGTGCTGCTGTCTACCTTTCT
CGGAGGCCTGGTGCCTAGAGGCTCTCACCACCATCATCACCACTCCGCTTGGTCCCATCCTCAGTTCG
AGAAGTGA
SEQ ID NO: 37
L7F_A1_33 coding nucleotide sequence, codon optimized
ATGTCTTGGAAGGTGGTCATCATCTTCTCCCTGCTGATCACCCCTCAGCACGGCCTGAAAGAGTCCTA
CCTGGAAGAGAGCTGCTCCACCATCACCGAGGGCTACCTGTCTGTGCTGAGAACCGGCTGGTACACCA
ACGTGTTCATGCTGTGTGTGGGCGACGTGGAAAACCTGACCTGTGCTGATGGCCCCAGCCTGCTGAAA
ACAGAGCTGGACCTGACCAAGAGCGCCCTGAGAGAACTGAGGACCGTGTCTGCAGATCAGCTGGCCAG
AGAGGAACAGATCGAGCAGCCTAGACAGTCCGGATGTGGTGCTGGTGCTACAGCTGGCGTGGCCATTG
CCAAGACCATCCGGCTGGAATCTGAAGTGACCGCCATCAAGAATGCCCTGAAAAAGACCAACGAGGCC
GTGTCTACCCTCGGCAATGGCGTTAGAGTGCTGGCCACAATGGTCCGAGAGCTGTGCGACTTCGTGTC
CAAGAATCTGACCCGGGCCATCAACAAGAACAAGTGTGATATCGCCGACCTGAAGATGGCCGTGTCCT
TCAGCCAGTTCAACCGGCGGTTCCTGAATGTCGTGCGGCAGTTCTCTGACAACGCCGGCATCACCCCT
GCCATCAGCCTGGATCTGATGACCGATGCCGAGCTGGCTAGAGCCGTGTCCAACATGCCTACCTCTGC
CGGCCAGATCAAGCTGATGCTCGAGAACAGAGCTATGGTCCGACGGAAAGGCTTCGGCTTCCTGATCG
GCGTGTACGGCTCTGACGTGATCTACATGGTGCAGCTGCCTATCTTCGGCGTGATCGACACCCCTTGC
TGGATCGTGAAGGCCGCTCCTAGCTGCTCTGAGAAGAAGGGCAACTACGCCTGCCTGCTGAGAGAGGA
CCAAGGCTGGTACTGTCAGAACGCCGGCTCCACCGTGTACTACCCCAACGAGAAGGACTGCGAGACAA
GAGGCGACCACGTGTTCTGCGATACCTGCGCTGGCATCAATGTGGCCGAGCAGTCCAAAGAGTGCAAC
ATCAACATCTCCACCACCAACTATCCCTGCAAGGTGTCCACCGGCAGACACCCCATTTCCATGGTGGC
TCTGTCTCCACTGGGTGCCCTGGTGGCTTGTTATAAGGGCGTGTCCTGCTCCATCGGCTCCAACAGAG
TGGGCATCATCAAGCAGCTGAACAAGGGCTGCAGCTACATCACCAACCAGGACGCCGATACCGTGACC
ATCGACAATACCGTGTATCAGCTGTCCAAGGTGGAAGGCGAGCAGCACGTGATCAAGGGCAGACCTGT
GTCCTCCAGCTTCGACCCCGTGAAGTTCCCTGAGGATCAGTTCAACGTGGCCCTGGACCAGGTGTTCG
AGTCCATCGAGAACTCTCAGGCTCTGGTGGACCAGTCCAACAGATGCTGTTCCGCCGGCTACATCCCC
GAGGCTCCTAGAGATGGACAGGCCTACGTCAGAAAGGACGGCGAATGGGTGCTGCTGTCTACCTTTCT
CGGAGGCCTGGTGCCTAGAGGCTCTCACCACCATCATCACCACTCCGCTTGGTCCCATCCACAGTTCG
AGAAGTGA
SEQ ID NO: 38
L7F_A1_4.2 coding nucleotide sequence, codon optimized
ATGTCTTGGAAGGTGGTCATCATCTTCTCCCTGCTGATCACCCCTCAGCACGGCCTGAAAGAGTCCTA
CCTGGAAGAGAGCTGCTCCACCATCACCGAGGGCTACCTGTCTGTGCTGAGAACCGGCTGGTACACCA
ACGTGTTCATGCTGGAAGTGGGCGACGTGGAAAACCTGACCTGTGCTGATGGCCCCAGCCTGATCAAG
ACCGAGCTGGACCTGACCAAGTCTGCCCTGAGAGAACTGAGGACCGTGTCTGCCGATCAGCTGGCCAG
AGAGGAACAGATCGAGCAGCCTAGACAGTCCGGATGTGGTGCTGGTGCTACAGCTGGCGTGGCCATTG
CCAAGACCATCCGGCTGGAATCTGAAGTGACCGCCTGGAAGAACGCCCTGAAAAAGACCAACGAGGTG
GTGTCTACCCTCGGCAACGGCGTCAGAGTGCTGGTCACAATGGTCCGAGAGCTGAAGGACTTCGTGTC
CAAGAACCTGACCAGGGCTCTGAACAAGAACAAGTGTGATATCGCCGACCTGAAGATGGCCGTGTCTT
TCAGCCAGTTCAACCGGCGGTTCCTGAACGTCGTGCGGCAGTTCTCTGATAACGCCGGCATCACCCCT
GCCATCAGCCTGGATCTGATGACCGATGCCGAGCTGGCTAGAGCCGTGTCCAACATGCCTACCTCTGC
CGGCCAGATCAAGCTGATGCTGGAAAACAGAGCCATGGTCCGACGGAAAGGCTTCGGCTTTCTGATCG
GCGTGTACGGCTCCTCCGTGATCTACATGGTGCAGCTGCCTATCTTCGGCGTGATCGACACCCCTTGC
TGGATCGTGAAGGCCGCTCCTAGCTGCTCTGAGAAGAAGGGCAACTACGCCTGCCTGCTGAGAGAGGA
CCAAGGCTGGTACTGTCAGAACGCCGGCTCCACCGTGTACTACCCCAACGAGAAGGACTGCGAGACAA
GAGGCGACCACGTGTTCTGCGATACCTGCGCTGGCATCAATGTGGCCGAGCAGTCCAAAGAGTGCAAC
ATCAACATCTCCACCACCAACTATCCCTGCAAGGTGTCCACCGGCAGGCACCCTATTTCCATGGTGGC
TCTGTCTCCACTGGGCGCCCTGGTGGCTTGTTATAAGGGCGTGTCCTGCTCCATCGGCTCCAACAGAG
TGGGCATCATCAAGCAGCTGAACAAGGGCTGCAGCTACATCACCAACCAGGACGCCGATACCGTGACC
ATCGACAATACCGTGTATCAGCTGTCCAAGGTGGAAGGCGAGCAGCACGTGATCAAGGGCAGACCTGT
GTCCTCCAGCTTCGACCCCGTGAAGTTCCCTGAGGATCAGTTCAACGTGGCCCTGGACCAGGTGTTCG
AGTCCATCGAGAACTCTCAGGCTCTGGTGGACCAGTCCAACCGGATCCTGTCCTCTGCCGAGTCTGCT
ATCGGCGGCTATATCCCCGAGGCTCCTAGAGATGGCCAGGCCTATGTTCGGAAGGATGGCGAATGGGT
GCTGCTGTCTACCTTCCTCGGAGGCCTGGTGCCTAGAGGCTCTCACCACCATCATCACCACTCCGCTT
GGTCCCATCCACAGTTCGAGAAGTGA
SEQ ID NO: 39
SF_A1_K-E294 coding nucleotide sequence, codon optimized
ATGTCTTGGAAGGTGGTCATCATCTTCTCCCTGCTGATCACCCCTCAGCACGGCCTGAAAGAGTCCTA
CCTGGAAGAGAGCTGCTCCACCATCACCGAGGGCTACCTGTCTGTGCTGAGAACCGGCTGGTACACCA
ACGTGTTCACCCTGGAAGTGGGCGACGTGGAAAACCTGACCTGTGCTGATGGCCCCAGCCTGATCAAG
ACCGAGCTGGACCTGACCAAGTCTGCCCTGAGAGAACTGAGGACCGTGTCTGCCGATCAGCTGGCCAG
AGAGGAACAGATCGAGAACCCTCGGCAGTCCAGATTCGTGCTGGGAGCTATTGCTCTGGGCGTGTGTA
CAGCCGCTGCTGTGACAGCTGGTGTCGCTATCGCCAAGACCATCCGGCTGGAATCTGAAGTGACCGCC
ATCAAGAACGCCCTGAAAAAGACCAACGAGGCCGTGTCCACACTCGGCAATGGCGTTAGAGTGCTGGC
CTTTGCTGTGCGCGAGCTGAAGGACTTCGTGTCCAAGAACCTGACCAGGGCTCTGAACAAGAACAAGT
GTGATATCGCCGACCTGAAGATGGCCGTGTCTTTCAGCCAGTTCAACCGGCGGTTCCTGAACGTCGTG
CGGCAGTTCTCTGATAACGCCGGCATCACCCCTGCCATCAGCCTGGATCTGATGACCGATGCCGAGCT
GGCTAGAGCCGTGTCTAACATGCCTACCTCTGCCGGCCAGATCAAGCTGATGCTGGAAAACAGAGCCA
TGGTCCGACGGAAAGGCTTCGGCTTTCTGATCGGCGTGTACGGCTCCTCCGTGATCTACATGGTGCAG
CTGCCTATCTTCGGCGTGATCGACACCCCTTGCTGGATCGTGAAGGCCGCTCCTAGCTGCTCTGAGAA
GAAGGGCAACTACGCCTGCCTGCTGAGAGAGGACCAAGGCTGGTACTGTCAGAACGCCGGCTCCACCG
TGTACTACCCCAACGAGAAGGACTGCGAGACAAGAGGCGACCACGTGTTCTGCGATACCGCCTGTGGC
ATCAATGTGGCCGAGCAGTCCAAAGAGTGCAACATCAACATCTCCACCACCAACTATCCCTGCAAGGT
GTCCACCGGCAGGCACCCTATTTCCATGGTGGCTCTGTCTCCACTGGGCGCCCTGGTGGCTTGTTATA
AGGGCGTGTCCTGCTCCATCGGCTCCAACAGAGTGGGCATCATCAAGCAGCTGAACAAGGGCTGCAGC
TACATCACCAACCAGGACGCCGATACCGTGACCATCGACAATACCGTGTATCAGCTGTCCAAGGTGGA
AGGCGAGCAGCACGTGATCAAGGGCAGACCTGTGTCCTCCAGCTTCGACCCCGTGAAGTTCCCTGAGG
ATCAGTTCAACGTGGCCCTGGACCAGGTGTTCGAGTCCATCGAGAACTCTCAGGCTCTGGTGGACCAG
TCCAACCGGATCCTGTCCTCTGCCGAGTCTGCTATCGGCGGCTATATCCCCGAGGCTCCTAGAGATGG
CCAGGCCTATGTTCGGAAGGATGGCGAATGGGTGCTGCTGTCTACCTTCCTCGGAGGCCTGGTGCCTA
GAGGCTCTCACCACCATCATCACCACTCCGCTTGGTCCCATCCACAGTTCGAGAAGTGA
SEQ ID NO: 40
sF_A1_MFur coding nucleotide sequence, codon optimized
ATGTCCTGGAAGGTCGTGATCATCTTCTCCCTGCTGATCACCCCCCAGCACGGCCTGAAAGAGTCCTA
CCTGGAAGAGAGCTGCTCCACCATCACCGAGGGCTACCTGTCTGTGCTGCGGACCGGCTGGTACACCA
ACGTGTTCACCCTGGAAGTGGGCGACGTGGAAAACCTGACCTGCGCCGATGGCCCCAGCCTGATCAAG
ACCGAGCTGGACCTGACCAAGTCCGCCCTGCGGGAACTGAGAACCGTGTCTGCCGATCAGCTGGCCAG
AGAGGAACAGATCGAGAACCCCCGGCAGTCCAAGAAACGGAAGCGGAGAGTGGCCACCGCCGCTGCTG
TGACAGCTGGCGTGGCCATTGCCAAGACCATCCGGCTGGAATCCGAAGTGACCGCCATCAAGAACGCC
CTGAAAAAGACCAACGAGGCCGTGTCTACCCTGGGCAATGGCGTGCGAGTGCTGGCTACAGCTGTGCG
CGAGCTGAAGGACTTCGTGTCCAAGAACCTGACCCGGGCCATCAACAAGAACAAGTGTGATATCGCCG
ACCTGAAGATGGCCGTGTCCTTTAGCCAGTTCAACCGGCGGTTCCTGAACGTCGTGCGGCAGTTCTCT
GACAACGCCGGCATCACCCCTGCCATCTCCCTGGATCTGATGACCGACGCCGAGCTGGCTAGAGCCGT
GTCCAACATGCCTACCTCTGCCGGCCAGATCAAGCTGATGCTGGAAAACCGGGCCATGGTGCGACGGA
AGGGCTTCGGCTTTCTGATCGGCGTGTACGGCTCCTCCGTGATCTACATGGTGCAGCTGCCTATCTTC
GGCGTGATCGACACCCCCTGCTGGATCGTGAAGGCCGCTCCTAGCTGCTCCGAGAAGAAGGGCAACTA
CGCCTGCCTGCTGAGAGAGGACCAGGGCTGGTACTGTCAGAACGCCGGCTCCACCGTGTACTACCCCA
ACGAGAAGGACTGCGAGACACGGGGCGACCACGTGTTCTGTGATACCGCTGCTGGCATCAACGTGGCC
GAGCAGTCCAAAGAGTGCAACATCAACATCTCCACCACCAACTACCCCTGCAAGGTGTCCACCGGCAG
GCACCCCATCTCTATGGTGGCCCTGTCTCCTCTGGGCGCCCTGGTGGCTTGTTACAAGGGCGTGTCCT
GCTCCATCGGCTCCAACAGAGTGGGCATCATCAAGCAGCTGAACAAGGGCTGCAGCTACATCACCAAC
CAGGACGCCGACACCGTGACCATCGACAATACCGTGTATCAGCTGTCCAAGGTGGAAGGCGAGCAGCA
CGTGATCAAGGGCAGACCCGTGTCCTCCAGCTTCGACCCCGTGAAGTTCCCCGAGGATCAGTTCAATG
TGGCCCTGGACCAGGTGTTCGAGTCCATCGAGAACTCCCAGGCTCTGGTGGACCAGTCCAACCGGATC
CTGTCCTCTGCCGAGAAGGGAAACACCTCCGGCAGAGAGAACCTGTATTTTCAAGGCGGCGGAGGCTC
CGGCTACATCCCTGAGGCTCCTAGAGATGGCCAGGCCTACGTGCGGAAGGATGGCGAATGGGTGCTGC
TGTCCACCTTCCTGGGCGGCATCGAGGGCAGACACCACCATCATCACCACTGA
SEQ ID NO: 41
M protein sequence from CAN97-83 strain (accession number Q6WB99) with purification tags
MGHHHHHHHHHHSSGHIDDDDKQESYLVDTYQGIPYTAAVQVDLVEKDLLPASLTIWFPLFQANTPPA
VLLDQLKTLTITTLYAASQSGPILKVNASAQGAAMSVLPKKFEVNATVALDEYSKLEFDKLTVCEVKT
VYLTTMKPYGMVSKFVSSAKPVGKKTHDLIALCDEMDLEKNTPVTIPAFIKSVSIKESESATVEAAIS
SEADQALTQAKIAPYAGLIMIMTMNNPKGIFKKLGAGTQVIVELGAYVQAESISKICKTWSHQGTRYV
LKSR
SEQ ID NO: 42
CCKQTNECCKNLERAVSA
SEQ ID NO: 43
CCRELKECCKNLENAVSA
SEQ ID NO: 44
CCRELKDCCKNLENAVSA
SEQ ID NO: 45
CCRELKDCCKNLERAVSA
SEQ ID NO: 46
CCRELKDCCKQLNKAVSA
SEQ ID NO: 47
CCRELKECCKQLNKAVSA
SEQ ID NO: 48
sF_B1_M coding nucleotide sequence, codon optimized
ATGATCATTATCTCCCTGCTGATCACCCCCCAGCACGGCCTGAAAGAGTCCTACCTGGAAGAGAGCTG
CTCCACCATCACCGAGGGCTACCTGTCTGTGCTGCGGACCGGCTGGTACACCAACGTGTTCACCCTGG
AAGTGGGCGACGTGGAAAACCTGACCTGCACCGATGGCCCCAGCCTGATCAAGACCGAGCTGGACCTG
ACCAAGTCCGCCCTGCGCGAGCTGAAAACCGTGTCTGCCGATCAGCTGGCCAGAGAGGAACAGATCGA
GAACCCCCGGCAGTCCAAGAAACGGAAGCGGAGAGTGGCCACCGCCGCTGCTGTGACAGCTGGAATCG
CTATCGCCAAGACCATCCGGCTGGAATCCGAAGTGAACGCCATCAAGGGCGCTCTGAAGCAGACCAAC
GAGGCCGTGTCTACCCTGGGCAATGGCGTGCGAGTGCTGGCTACAGCTGTGCGGGAACTGAAAGAATT
CGTGTCCAAGAACCTGACCAGCGCCATCAACCGGAACAAGTGTGATATCGCCGACCTGAAGATGGCCG
TGTCCTTCAGCCAGTTCAACCGGCGGTTCCTGAACGTCGTGCGGCAGTTCTCTGACAACGCCGGCATC
ACCCCTGCCATCTCCCTGGATCTGATGACCGACGCCGAGCTGGCTAGAGCCGTGTCTTACATGCCTAC
CTCTGCCGGCCAGATCAAGCTGATGCTGGAAAACCGGGCCATGGTGCGACGGAAGGGCTTCGGCATCC
TGATCGGCGTGTACGGCTCCTCCGTGATCTACATGGTGCAGCTGCCTATCTTCGGCGTGATCGACACC
CCCTGCTGGATTATCAAGGCCGCTCCCAGCTGCTCCGAGAAGAACGGCAACTACGCCTGCCTGCTGAG
AGAGGACCAGGGCTGGTACTGCAAGAACGCCGGCTCCACCGTGTACTACCCCAACGAGAAGGACTGCG
AGACACGGGGCGACCACGTGTTCTGTGATACCGCTGCTGGCATCAACGTGGCCGAGCAGTCCAGAGAG
TGCAACATCAACATCTCCACCACCAACTACCCCTGCAAGGTGTCCACCGGCAGGCACCCCATCTCTAT
GGTGGCCCTGTCTCCTCTGGGAGCCCTGGTGGCTTGTTACAAGGGCGTGTCCTGCTCCATCGGCTCCA
ACTGGGTGGGAATCATCAAGCAGCTGCCCAAGGGCTGCAGCTACATCACCAACCAGGACGCCGACACC
GTGACCATCGACAATACCGTGTATCAGCTGTCCAAGGTGGAAGGCGAGCAGCACGTGATCAAGGGCAG
ACCCGTGTCCAGCTCCTTCGACCCCATCAAGTTCCCCGAGGATCAGTTCAATGTGGCCCTGGACCAGG
TGTTCGAGTCCATCGAGAACTCCCAGGCTCTGGTGGACCAGTCCAACAAGATCCTGAACTCCGCCGAG
AAGGGCAACACCTCCGGCAGAGAGAACCTGTATTTTCAAGGCGGCGGAGGCTCCGGCTACATCCCTGA
GGCTCCTAGAGATGGCCAGGCCTACGTGCGGAAGGATGGCGAATGGGTGCTGCTGTCCACCTTCCTGT
GA
SEQ ID NO: 50
>SF_B1_K-L7
MSWKVMIIISLLITPQHGLKESYLEESCSTITEGYLSVLRTGWYTNVFTLEVGDVENLTCTDGPSLIKTELDLTK
SALRELKTVSADQLAREEQIEQPRQSGCGAGATAGIAIAKTIRLESEVNAIKGALKQTNEAVSTLGNGVRVLAFA
VRELKEFVSKNLTSALNRNKCDIADLKMAVSFSQFNRRFLNVVRQFSDNAGITPAISLDLMTDAELARAVSYMPT
SAGQIKLMLENRAMVRRKGFGILIGVYGSSVIYMVQLPIFGVIDTPCWIIKAAPSCSEKNGNYACLLREDQGWYC
KNAGSTVYYPNEKDCETRGDHVFCDTCAGINVAEQSRECNINISTTNYPCKVSTGRHPISMVALSPLGALVACYK
GVSCSIGSNWVGIIKQLPKGCSYITNQDADTVTIDNTVYQLSKVEGEQHVIKGRPVSSSFDPIKFPEDQFNVALD
QVFESIENSQALVDQSNKILNSAESAIGGYIPEAPRDGQAYVRKDGEWVLLSTFLGGLVPRGSHHHHHHSAWSHP
QFEK
SEQ ID NO: 51
>L7F_B1_31
MSWKVMIIISLLITPQHGLKESYLEESCSTITEGYLSVLRTGWYTNVEMLEVGDVENLTCTDGPSLLKTELDLTK
SALRNLKTVSADQLAREEQIEQPRQSGCGAGATAGIAIAKTIRLESEVNAIKGALKQTNEAVSTLGNGVRVLATM
VRELKEFVSKNLTSAINRNKCDIADLKMAVSFSQFNRRFLNVVRQFSDNAGITPAISLDLMTDAELARAVSYMPT
SAGQIKLMLENRAMVRRKGFGILIGVYGSSVIYMVQLPIFGVIDTPCWIIKAAPSCSEKNGNYACLLREDQGWYC
KNAGSTVYYPNEKDCETRGDHVFCDTCAGINVAEQSRECNINISTTNYPCKVSTGRHPISMVALSPLGALVACYK
GVSCSIGSNWVGIIKQLPKGCSYITNQDADTVTIDNTVYQLSKVEGEQHVIKGRPVSSSFDPIKFPEDQFNVALD
QVFESIENSQALVDQSNKILNAGYIPEAPRDGQAYVRKDGEWVLLSTFLGGLVPRGSHHHHHHSAWSHPQFEK
SEQ ID NO: 52
>L7F_B1_33
MSWKVMIIISLLITPQHGLKESYLEESCSTITEGYLSVLRTGWYTNVFMLCVGDVENLTCTDGPSLLKTELDLTK
SALRELKTVSADQLAREEQIEQPRQSGCGAGATAGIAIAKTIRLESEVNAIKGALKQTNEAVSTLGNGVRVLATM
VRELCEFVSKNLTSAINRNKCDIADLKMAVSFSQFNRRFLNVVRQFSDNAGITPAISLDLMTDAELARAVSYMPT
SAGQIKLMLENRAMVRRKGFGILIGVYGSDVIYMVQLPIFGVIDTPCWIIKAAPSCSEKNGNYACLLREDQGWYC
KNAGSTVYYPNEKDCETRGDHVFCDTCAGINVAEQSRECNINISTTNYPCKVSTGRHPISMVALSPLGALVACYK
GVSCSIGSNWVGIIKQLPKGCSYITNQDADTVTIDNTVYQLSKVEGEQHVIKGRPVSSSFDPIKFPEDQFNVALD
QVFESIENSQALVDQSNKCCNAGYIPEAPRDGQAYVRKDGEWVLLSTFLGGLVPRGSHHHHHHSAWSHPQFEK
SEQ ID NO: 53
>L7F_B1_4.2
MSWKVMIIISLLITPQHGLKESYLEESCSTITEGYLSVLRTGWYTNVEMLEVGDVENLTCTDGPSLIKTELDLTK
SALRELKTVSADQLAREEQIEQPRQSGCGAGATAGIAIAKTIRLESEVNAWKGALKQTNEVVSTLGNGVRVLVTM
VRELKEFVSKNLTSALNRNKCDIADLKMAVSFSQFNRRFLNVVRQFSDNAGITPAISLDLMTDAELARAVSYMPT
SAGQIKLMLENRAMVRRKGFGILIGVYGSSVIYMVQLPIFGVIDTPCWIIKAAPSCSEKNGNYACLLREDQGWYC
KNAGSTVYYPNEKDCETRGDHVFCDTCAGINVAEQSRECNINISTTNYPCKVSTGRHPISMVALSPLGALVACYK
GVSCSIGSNWVGIIKQLPKGCSYITNQDADTVTIDNTVYQLSKVEGEQHVIKGRPVSSSFDPIKFPEDQFNVALD
QVFESIENSQALVDQSNKILNSAESAIGGYIPEAPRDGQAYVRKDGEWVLLSTFLGGLVPRGSHHHHHHSAWSHP
QFEK

1. An immunogenic composition consisting essentially of a stabilized pre-fusion conformation form of the human metapneumovirus (hMPV) F protein or fragment thereof as the only hMPV antigen and optionally one or more adjuvants and/or at least one pharmaceutically exactable carrier or excipient; wherein said hMPV protein is derived from one subgroup of genotype A or B, and wherein said immunogenic composition cross-neutralizes the hMPV from another subgroup and/or genotype.

2. The composition of claim 1, wherein the stabilized pre-fusion conformation form of the human metapneumovirus (hMPV) F protein or fragment thereof is of the A1 subgroup.

3. The composition of claim 1-2, wherein the composition consists essentially of i) a stabilized pre-fusion conformation form of the human metapneumovirus (hMPV) F protein or fragment thereof of the A genotype and ii) a stabilized pre-fusion conformation form of the human metapneumovirus (hMPV) F protein or fragment thereof of the B genotype; and optionally one or more adjuvants and/or at least one pharmaceutically exactable carrier or excipient; wherein said immunogenic composition cross-neutralizes the other subgroup and/or other genotype.

4. The composition of claim 3, wherein the stabilized pre-fusion conformation form of the human metapneumovirus (hMPV) F protein or fragment thereof of the A genotype is of the A1 subgroup and wherein the stabilized pre-fusion conformation form of the human metapneumovirus (hMPV) F protein or fragment thereof of the B genotype is of the B1 subgroup.

5. The immunogenic composition of any preceding claim, wherein the pre-fusion F protein is the recombinant protein.

6. The immunogenic composition of any preceding claim, wherein the pre-fusion F protein lacks the cytoplasmic tail and/or transmembrane domain.

7. The immunogenic composition of any preceding claim, wherein the pre-fusion F protein has an amino acid sequence, which is a modified amino acid sequence of the native F protein derived from the hMPV strain or clinical isolate.

8. The immunogenic composition of claim 9, wherein the native F protein sequence is selected from the group consisting of the amino acid sequences of SEQ ID NO: 1 to 10 that are derived from the hMPV strains NL/1/00, NL/17/00, TN/94-49, NCL174, CAN97-83, NL/1/9, NDL00-1, C1-334, CAN97-82 and TN/89-515.

9. The immunogenic composition of any preceding claim, wherein the pre-fusion F protein comprises at least one mutation (substitution or deletion), preferably up to 10 mutations, relative to the native F protein sequence of SEQ ID NO: 1 to 10.

10. The immunogenic composition of any preceding claim, wherein the pre-fusion F protein comprises one or more amino acid substitution(s) to cysteine, which introduce one or more non-native disulfide bond(s) that stabilize the pre-fusion conformation.

11. The immunogenic composition of claim 10, wherein the cysteine substitution is introduced at
any one of positions 103-120 and any one of positions 335-345;
any one of positions 107-118 and any one of positions 335-342;
any one of positions 117-129 and any one of positions 256-261;
any one of positions 87-102 and any one of positions 117-127;
any one of positions 102-113 and any one of positions 117-127;
any one of positions 102-113 and any one of positions 87-102;
any one of positions 337-341 and any one of positions 421-426;
any one of positions 112-120 and any one of positions 424-432;
any one of positions 150-156 and any one of positions 392-400;
any one of positions 112-120 and any one of positions 370-377;
any one of positions 365-375 and any one of positions 455-465;
any one of positions 365-375 and any one of positions 105-115; or
any one of positions 60-70 and any one of positions 175-185,
wherein the positions corresponds to the amino acids of the native F protein sequence of SEQ ID NO: 1 to 10.

12. The immunogenic composition of any preceding claim, wherein the pre-fusion F protein consists of a single polypeptide chain stabilized by at least one non-natural disulfide bond.

13. The immunogenic composition of claim 12, wherein the single-chain pre-fusion F protein lacks a protease cleavage site between F1 and F2 domains relative to the native F protein.

14. The immunogenic composition of claims 12 and 13, wherein the single-chain pre-fusion F protein comprises a substitution of arginine at position 102 relative to the amino acid positions of the native F protein for another amino acid, preferably glycine.

15. The immunogenic composition of claims 12 to 14, wherein the amino acid residues at positions 103-118 of the native F protein are replaced with a heterologous linker consisting of 1 to 5 amino acid residues including cysteine residue, wherein said cysteine residue forms a disulfide bond with a cysteine residue in the F1 domain.

16. The immunogenic composition of claim 15, wherein the heterologous linker comprises at least one alanine, glycine or valine residue, preferably the linker has the sequence CGAGA or CGAGV.

17. The immunogenic composition of claims 12 to 16, wherein the pre-fusion F protein comprises one or more substitution(s) at positions corresponding to positions 49, 51, 67, 80, 137, 147, 159, 160, 161, 166, 177, 258, 266, 480 and/or 481 of the native hMPV F protein.

18. The immunogenic composition of claim 17, wherein the substitution is selected from the group consisting of T49M, E80N, I137W, A147V, A159V, T160F, A161M, I67L, I177L, F258I, S266D, I480C and/or L481C.

19. The immunogenic composition of claims 12 to 18, wherein the single-chain pre-fusion F protein comprises one of the following substitution combinations:
N97Q, R102G and G294E;
N97Q, R102G, T160F, I177L and G294E;
N97Q, R102G, T49M, I67L, A161M, E80N, F258I and G294E;
N97Q, R102G, T49M, I67L, A161M, E51C, K166C, S266D, G294E, I480C and L481C; or
N97Q, R102G, T49M, A161M, I137W, A159V, A147V, I177L and G294E.

20. The immunogenic composition of any of claims 12 to 19, wherein the pre-fusion F protein comprises or consists of the amino acid sequence of SEQ ID NO: 11 (L7F_A1_23)

21. The immunogenic composition of any of claims 12 to 19, wherein the pre-fusion F protein comprises or consists of the amino acid sequence of SEQ ID NO: 12 (L7F_B1_23).

22. The immunogenic composition of any of claims 12 to 19, wherein the pre-fusion F protein comprises or consists of the amino acid sequence of SEQ ID NO: 13 (L7F_A1_23.2).

23. The immunogenic composition of any of claims 12 to 19, wherein the pre-fusion F protein comprises or consists of the amino acid sequence of SEQ ID NO: 14 (L7F_B1_23.2).

24. The immunogenic composition of any of claims 12 to 19, wherein the pre-fusion F protein comprises or consist of the amino acid sequence of SEQ ID NO: 15 (sF_A1_K_L7).

25. The immunogenic composition of any of claims 12 to 19, wherein the pre-fusion F protein comprises or consists of the amino acid sequence of SEQ ID NO: 16 (L7F_A1_31).

26. The immunogenic composition of any of claims 12 to 19, wherein the pre-fusion F protein comprises or consists of the amino acid sequence of SEQ ID NO: 17 (L7F_A1_33).

27. The immunogenic composition of any of claims 12 to 19, wherein the pre-fusion F protein comprises or consists of the amino acid sequence of SEQ ID NO: 18 (construct L7F_A1_4.2).

28. The immunogenic composition of any of claims 12 to 19, wherein the pre-fusion F protein comprises or consists of the amino acid sequence of SEQ ID NO: 50 (construct sF_B1_K_L7).

29. The immunogenic composition of any of claims 12 to 19, wherein the pre-fusion F protein comprises or consists of the amino acid sequence of SEQ ID NO: 51 (construct L7F_B1_31).

30. The immunogenic composition of any of claims 12 to 19, wherein the pre-fusion F protein comprises or consists of the amino acid sequence of SEQ ID NO: 52 (construct L7F_B1_33).

31. The immunogenic composition of any of claims 12 to 19, wherein the pre-fusion F protein comprises or consists of the amino acid sequence of SEQ ID NO: 53 (construct L7F_B1_4.2).

32. The immunogenic composition of any of claims 1 to 11, wherein the pre-fusion F protein is a two-polypeptide-chain protein and comprises or consists of the amino acid sequence of SEQ ID NO: 19.

33. The immunogenic composition of any of claims 1 to 11, wherein the pre-fusion F protein is a two-polypeptide-chain protein and comprises or consists of the amino acid sequence of SEQ ID NO: 20.

34. The immunogenic composition of any of claims 1 to 11, wherein the stabilized post-fusion F protein comprises the deletion of the amino acid residues at positions 103 to 111, replacement of R102 by a linker KKRKRR and the substitution G294E relative to the amino acid positions of the native F protein.

35. The immunogenic composition of any of claims 1 to 34, wherein the pre- -fusion F protein: i) comprises the amino acid sequence having at least 80% sequence identity to the amino acid sequence selected from the group consisting of SEQ ID NO: 1 to 20, and ii) its immunogenicity is similar to immunogenicity of the parental F protein of SEQ ID NO: 1 to 20.

36. The immunogenic composition of any of claims 1 to 34, wherein the pre- -fusion F protein i) comprises the amino acid sequence having at least 90% sequence identity to the amino acid sequence selected from the group consisting of SEQ ID NO: 1 to 20, and ii) its immunogenicity is equal or similar to immunogenicity of the parental F protein of SEQ ID NO: 1 to 20.

37. The immunogenic composition of any of claims 1 to 34, wherein the pre- -fusion F protein i) comprises the amino acid sequence having at least 95% sequence identity to the amino acid sequence selected from the group consisting of SEQ ID NO: 1 to 20, and ii) its immunogenicity is equal or similar to immunogenicity of the parental F protein of SEQ ID NO: 1 to 20.

38. The immunogenic composition of any preceding claim, wherein the pre-fusion hMPV F protein comprises a trimerization helper domain (foldon) having the sequence of SEQ ID NO: 23 to 28 or a variant thereof.

39. The immunogenic composition of any preceding claim, wherein the F protein is produced as a homo- or hetero-trimer.

40. The immunogenic composition of any preceding claim, wherein the composition comprises a further non-hMPV antigen.

41. The immunogenic composition of any preceding claim, wherein the adjuvant is selected from the group consisting of alum, CpG, such as CpG1018, ODN, I-ODN, IC31®, MF59®, AddaVax™, AS03, AS01, QS21, MPL, GLA-SE, GLA-3M-052-LS, 3M-052-alum or combinations thereof.

42. The immunogenic composition of any preceding claim, wherein the adjuvant consists of two or more adjuvants that are selected from the group consisting of alum, CpG, such as CpG1018, ODN, I-ODN, IC31®, MF59®, AddaVax™, AS03, AS01, QS21, MPL, GLA-SE, GLA-3M-052-LS and 3M-052-alum.

43. The immunogenic composition of any preceding claim, wherein the adjuvant is alum.

44. The immunogenic composition of any preceding claim, wherein the adjuvant is IC31®.

45. The immunogenic composition of any preceding claim, wherein the adjuvant is GLA-SE.

46. The immunogenic composition of any preceding claim, wherein the adjuvant is 3M-052-alum.

47. immunogenic composition of any preceding claim, wherein the adjuvant is GLA-3M-052-LS.

48. The immunogenic composition of any preceding claim, wherein the adjuvant consists of alum and CpG1018.

49. The immunogenic composition of any preceding claim, wherein the adjuvant consists of alum and MPL.

50. The immunogenic composition of any preceding claim, wherein the adjuvant consists of alum and IC31®.

51. The immunogenic composition of any preceding claim, wherein the adjuvant is AddaVax™.

52. The immunogenic composition of any preceding claim, wherein the composition is capable to elicit neutralizing antibodies against the pre-fusion F protein.

53. The immunogenic composition of any preceding claim, wherein the composition comprising the pre-fusion protein or the pre- and pre-fusion protein provides a superior immune response (neutralizing antibody titers) as compare to immune response (neutralizing antibody titers) elicited by a composition comprising the post-fusion F protein used at the same total protein amount.

54. The immunogenic composition of any preceding claim, wherein the composition provides protection against more than one hMPV strain.

55. The immunogenic composition of any preceding claim, wherein the composition provides protection against the hMPV strains of genotype A.

56. The immunogenic composition of any preceding claim, wherein the composition provides protection against the hMPV strains of genotype B.

57. The immunogenic composition of any preceding claims, wherein the composition provides protection against the hMPV strains of genotype A and genotype B.

58. The immunogenic composition of any preceding claim, wherein the composition is a vaccine.

59. The immunogenic composition according to any preceding claim for use as a medicament.

60. The immunogenic composition according to any preceding claim for treating and/or preventing hMPV infection and associated disease in a subject.

61. A method for generating an immune response to the hMPV F protein in a subject, wherein the method comprises administering to the subject an effective amount of the immunogenic composition according to any previous claims 1 to 60.

62. The method of claim 61, wherein the immunogenic composition is administered intramuscularly, intradermally, subcutaneously, mucosally, intrarectally, or orally.

63. The method of claims 61 and 62, wherein the method comprises a prime-boost administration of the immunogenic composition according to any of claims 1 to 55, wherein the prime-boost is done with the same immunogenic composition.

64. The method of claims 61 and 63, wherein the method comprises a prime-boost administration of the immunogenic composition according to any of claims 1 to 55, wherein the prime administration is done with the composition comprising the F protein of the genotype A and the boost administration is done with the composition comprising the F protein of the genotype B, or vise versa.

65. A method for treating and/or preventing hMPV infection in a subject, wherein the method comprises administering to the subject a therapeutically effective amount of the immunogenic composition according to any of claims 1 to 55 in order to generate neutralizing antibodies against the pre-fusion hMPV F protein and provide protection against the hMPV strains of at least one genotype A or B, preferably both.

66. A method for producing the immunogenic composition according to any of claims 1 to 60, wherein the method comprises i) expression of the recombinant pre-fusion F protein from the corresponding nucleic acid molecule inserted in an expression vector in a host cell, ii) purifying the expressed recombinant F protein; and iii) combining the purified recombinant protein with a pharmaceutically acceptable carrier and/or excipient, optionally with an adjuvant.


SEQUENCE LISTING (53 sequences)

SEQ ID NO: 1 - protein, 539 aa, Human metapneumovirus
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 2 - protein, 539 aa, Human metapneumovirus
MSWKVVIIFSLLITPQHGLKESYLEESCSTITEGYLSVLRTGWYTNVFTLEVGDVENLTCADGPSLIKTELELTKSALRELKTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSKKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNTGFIIVIILIAVLGSSMILVSIFIIIKKTKKQTGAPPELSGVTNNGFIPHS

SEQ ID NO: 3 - protein, 539 aa, Human metapneumovirus
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYTVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 4 - protein, 539 aa, Human metapneumovirus
MSWKVVIIFSLLITPQHSLKESYLEESCSTITEGYLSVLRTGWYTNVFTLEVGDVENLTCADGPSLIKTELDLTKSALRELKPVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTAAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNTGFIIVIILIAVLGSSMILVSVFIIIKKTRKPTGAPPELSGVTNNGFIPHS

SEQ ID NO: 5 - protein, 539 aa, Human metapneumovirus
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 6 - protein, 536 aa, Human metapneumovirus
MSWKVMIIISLLITPQHGLKESYLEESCSTITEGYLSVLRTGWYTNVFTLEVGDVENLTCPSLIKTELDLTKSALRELKTVSADQLAREEQIENPRQSRFVLGAIALGVATAAAVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNSAEKGNTGFIIVVILVAVLGLTMISVSIIIIIKKTRKPTGAPPELNGVTNGGFIPHS

SEQ ID NO: 7 - protein, 539 aa, Human metapneumovirus; identical to SEQ ID NO: 1

SEQ ID NO: 8 - protein, 539 aa, Human metapneumovirus
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNKKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAEKGNTGFIIVIILIAVLGLTMISVSIIIIIKKTRKPTGAPPELNGVTNGGFIPHS

SEQ ID NO: 9 - protein, 539 aa, Human metapneumovirus
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIRFPEDQFNVALDQVFESIENSQALVEQSNKILNSAEKGNTGFIIVIILVAVLGLTMISVSIIIIIKKTRKPTGAPPELNGVTNGGFIPHS

SEQ ID NO: 10 - protein, 539 aa, Human metapneumovirus; identical to SEQ ID NO: 8

SEQ ID NO: 11 - protein, 523 aa, Artificial sequence
  Synthetic
MSWKVVIIFSLLITPQHGLKESYLEESCSTITEGYLSVLRTGWYTNVFTLEVGDVENLTCADGPSLIKTELDLTKSALRELRTVSADQLAREEQIEQPRQSGCGAGATAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSAGYIPEAPRDGQAYVRKDGEWVLLSTFLGGLVPRGSHHHHHHSAWSHPQFEK

SEQ ID NO: 12 - protein, 523 aa, Artificial sequence
  Synthetic
MSWKVMIIISLLITPQHGLKESYLEESCSTITEGYLSVLRTGWYTNVFTLEVGDVENLTCTDGPSLIKTELDLTKSALRELKTVSADQLAREEQIEQPRQSGCGAGATAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTCAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNAGYIPEAPRDGQAYVRKDGEWVLLSTFLGGLVPRGSHHHHHHSAWSHPQFEK

SEQ ID NO: 13 - protein, 523 aa, Artificial sequence
  Synthetic
MSWKVVIIFSLLITPQHGLKESYLEESCSTITEGYLSVLRTGWYTNVFTLEVGDVENLTCADGPSLIKTELDLTKSALRELRTVSADQLAREEQIEQPRQSGCGAGVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSAGYIPEAPRDGQAYVRKDGEWVLLSTFLGGLVPRGSHHHHHHSAWSHPQFEK

SEQ ID NO: 14 - protein, 523 aa, Artificial sequence
  Synthetic
MSWKVMIIISLLITPQHGLKESYLEESCSTITEGYLSVLRTGWYTNVFTLEVGDVENLTCTDGPSLIKTELDLTKSALRELKTVSADQLAREEQIEQPRQSGCGAGVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTCAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNAGYIPEAPRDGQAYVRKDGEWVLLSTFLGGLVPRGSHHHHHHSAWSHPQFEK

SEQ ID NO: 15 - protein, 529 aa, Artificial sequence
  Synthetic
MSWKVVIIFSLLITPQHGLKESYLEESCSTITEGYLSVLRTGWYTNVFTLEVGDVENLTCADGPSLIKTELDLTKSALRELRTVSADQLAREEQIEQPRQSGCGAGATAGVAIAKTIRLESEVTAIKNALKKTNEAVSTLGNGVRVLAFAVRELKDFVSKNLTRALNKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAESAIGGYIPEAPRDGQAYVRKDGEWVLLSTFLGGLVPRGSHHHHHHSAWSHPQFEK

SEQ ID NO: 16 - protein, 523 aa, Artificial sequence
  Synthetic
MSWKVVIIFSLLITPQHGLKESYLEESCSTITEGYLSVLRTGWYTNVFMLEVGDVENLTCADGPSLLKTELDLTKSALRNLRTVSADQLAREEQIEQPRQSGCGAGATAGVAIAKTIRLESEVTAIKNALKKTNEAVSTLGNGVRVLATMVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSAGYIPEAPRDGQAYVRKDGEWVLLSTFLGGLVPRGSHHHHHHSAWSHPQFEK

SEQ ID NO: 17 - protein, 523 aa, Artificial sequence
  Synthetic
MSWKVVIIFSLLITPQHGLKESYLEESCSTITEGYLSVLRTGWYTNVFMLCVGDVENLTCADGPSLLKTELDLTKSALRELRTVSADQLAREEQIEQPRQSGCGAGATAGVAIAKTIRLESEVTAIKNALKKTNEAVSTLGNGVRVLATMVRELCDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSDVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRCCSAGYIPEAPRDGQAYVRKDGEWVLLSTFLGGLVPRGSHHHHHHSAWSHPQFEK

SEQ ID NO: 18 - protein, 529 aa, Artificial sequence
  Synthetic
MSWKVVIIFSLLITPQHGLKESYLEESCSTITEGYLSVLRTGWYTNVFMLEVGDVENLTCADGPSLIKTELDLTKSALRELRTVSADQLAREEQIEQPRQSGCGAGATAGVAIAKTIRLESEVTAWKNALKKTNEVVSTLGNGVRVLVTMVRELKDFVSKNLTRALNKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAESAIGGYIPEAPRDGQAYVRKDGEWVLLSTFLGGLVPRGSHHHHHHSAWSHPQFEK

SEQ ID NO: 19 - protein, 540 aa, Artificial sequence
  Synthetic
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVCTAAAVTAGVAIAKTIRLESEVTAIKNALKKTNEAVSTLGNGVRVLAFAVRELKDFVSKNLTRALNKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAESAIGGYIPEAPRDGQAYVRKDGEWVLLSTFLGGLVPRGSHHHHHHSAWSHPQFEK

SEQ ID NO: 20 - protein, 540 aa, artificial sequence
  Synthetic
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVCTAAAVTAGIAIAKTIRLESEVNAIKGALKQTNEAVSTLGNGVRVLAFAVRELKEFVSKNLTSALNRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTACGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNSAESAIGGYIPEAPRDGQAYVRKDGEWVLLSTFLGGLVPRGSHHHHHHSAWSHPQFEK

SEQ ID NO: 21 - protein, 538 aa, Artificial sequence
  Synthetic
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSKKRKRRVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGGGGSGYIPEAPRDGQAYVRKDGEWVLLSTFLGGIEGRHHHHHH

SEQ ID NO: 22 - protein, 550 aa, Artificial sequence
  Synthetic
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSKKRKRRVATAAAVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNSAEKGNTSGRENLYFQGGGGSGYIPEAPRDGQAYVRKDGEWVLLSTFLGGLVPRGSHHHHHHSAWSHPQFEK

SEQ ID NO: 23 - protein, 27 aa, Artificial sequence
  Synthetic
GYIPEAPRDGQAYVRKDGEWVLLSTFL

SEQ ID NO: 24 - protein, 27 aa, Artificial sequence
  Synthetic
GYIPEAPRNGTAYVRKDGEWVLLSTFL

SEQ ID NO: 25 - protein, 27 aa, Artificial sequence
  Synthetic
GYIPEAPRDGQAYVRKNGTWVLLSTFL

SEQ ID NO: 26 - protein, 27 aa, Artificial sequence
  Synthetic
GYIPEAPRDGQAYVRKDGNWTLLSTFL

SEQ ID NO: 27 - protein, 27 aa, Artificial sequence
  Synthetic
GYIPEAPRNGTAYVRKNGTWVLLSTFL

SEQ ID NO: 28 - protein, 27 aa, Artificial sequence
  Synthetic
GYIPEAPRNGTAYVRKDGNWTLLSTFL

SEQ ID NO: 29 - protein, 4 aa, Artificial sequence
  Synthetic
ILSA

SEQ ID NO: 30 - protein, 4 aa, Artificial sequence
  Synthetic
CCSA

SEQ ID NO: 31 - DNA, 1572 bp, Artificial sequence
  Synthetic
atgtcttggaaggtggtcatcatcttctccctgctgatcacccctcagcacggcctgaaagagtcctacctggaagagagctgctccaccatcaccgagggctacctgtctgtgctgagaaccggctggtacaccaacgtgttcaccctggaagtgggcgacgtggaaaacctgacctgtgctgatggccccagcctgatcaagaccgagctggacctgaccaagtctgccctgagagaactgaggaccgtgtctgccgatcagctggccagagaggaacagatcgagcagcctagacagtccggatgtggtgctggtgctacagctggcgtggccattgccaagaccatccggctggaatctgaagtgaccgccatcaagaacgccctgaaaaagaccaacgaggccgtgtctaccctcggcaatggcgttagagtgctggccacagccgtgcgcgagctgaaggatttcgtgtccaagaacctgaccagggccatcaacaagaacaagtgtgatatcgccgacctgaagatggccgtgtccttcagccagttcaaccggcggttcctgaatgtcgtgcggcagttctctgacaacgccggcatcacccctgccatcagcctggatctgatgaccgatgccgagctggctagagccgtgtccaacatgcctacctctgccggccagatcaagctgatgctggaaaacagagccatggtccgacggaaaggcttcggctttctgatcggcgtgtacggctcctccgtgatctacatggtgcagctgcctatcttcggcgtgatcgacaccccttgctggatcgtgaaggccgctcctagctgctctgagaagaagggcaactacgcctgcctgctgagagaggaccaaggctggtactgtcagaacgccggctccaccgtgtactaccccaacgagaaggactgcgagacaagaggcgaccacgtgttctgcgatacctgcgctggcatcaatgtggccgagcagtccaaagagtgcaacatcaacatctccaccaccaactatccctgcaaggtgtccaccggcaggcaccctatttccatggtggctctgtctccactgggcgccctggtggcttgttataagggcgtgtcctgctccatcggctccaacagagtgggcatcatcaagcagctgaacaagggctgcagctacatcaccaaccaggacgccgataccgtgaccatcgacaataccgtgtatcagctgtccaaggtggaaggcgagcagcacgtgatcaagggcagacctgtgtcctccagcttcgaccccgtgaagttccctgaggatcagttcaacgtggccctggaccaggtgttcgagtccatcgagaactctcaggctctggtggaccagtccaaccggattctgtctgccggctacatccccgaggctcctagagatggacaggcctacgtcagaaaggacggcgaatgggtgctgctgtctacctttctcggaggcctggtgcctagaggctctcaccaccatcatcaccactccgcttggtcccatccacagttcgagaagtga

SEQ ID NO: 32 - DNA, 1572 bp, Artificial sequence
  Synthetic
atgtcttggaaagttatgattattatttctttgttgattactccacaacatggtttgaaagaatcttatttggaagaatcttgttctactattactgaaggttatttgtctgttttgagaactggttggtatactaatgtttttactttggaagttggtgatgttgaaaatttgacttgtactgatggtccatctttgattaaaactgaattggatttgactaaatctgctttgagagaattgaaaactgtttctgctgatcaattggctagagaagaacaaattgaacaaccaagacaatctggttgtggtgctggtgctactgctggtattgctattgctaaaactattagattggaatctgaagttaatgctattaaaggtgctttgaaacaaactaatgaagctgtttctactttgggtaatggtgttagagttttggctactgctgttagagaattgaaagaatttgtttctaaaaatttgacttctgctattaatagaaataaatgtgatattgctgatttgaaaatggctgtttctttttctcaatttaatagaagatttttgaatgttgttagacaattttctgataatgctggtattactccagctatttctttggatttgatgactgatgctgaattggctagagctgtttcttatatgccaacttctgctggtcaaattaaattgatgttggaaaatagagctatggttagaagaaaaggttttggtattttgattggtgtttatggttcttctgttatttatatggttcaattgccaatttttggtgttattgatactccatgttggattattaaagctgctccatcttgttctgaaaaaaatggtaattatgcttgtttgttgagagaagatcaaggttggtattgtaaaaatgctggttctactgtttattatccaaatgaaaaagattgtgaaactagaggtgatcatgttttttgtgatacttgtgctggtattaatgttgctgaacaatctagagaatgtaatattaatatttctactactaattatccatgtaaagtttctactggtagacatccaatttctatggttgctttgtctccattgggtgctttggttgcttgttataaaggtgtttcttgttctattggttctaattgggttggtattattaaacaattgccaaaaggttgttcttatattactaatcaagatgctgatactgttactattgataatactgtttatcaattgtctaaagttgaaggtgaacaacatgttattaaaggtagaccagtttcttcttcttttgatccaattaaatttccagaagatcaatttaatgttgctttggatcaagtttttgaatctattgaaaattctcaagctttggttgatcaatctaataaaattttgaatgctggttatattccagaagctccaagagatggtcaagcttatgttagaaaagatggtgaatgggttttgttgtctacttttttgggtggtttggttccaagaggttctcatcatcatcatcatcattctgcttggtctcatccacaatttgaaaaatga

SEQ ID NO: 33 - DNA, 1572 bp, Artificial sequence
  Synthetic
atgtcttggaaagttgttattattttttctttgttgattactccacaacatggtttgaaagaatcttatttggaagaatcttgttctactattactgaaggttatttgtctgttttgagaactggttggtatactaatgtttttactttggaagttggtgatgttgaaaatttgacttgtgctgatggtccatctttgattaaaactgaattggatttgactaaatctgctttgagagaattgagaactgtttctgctgatcaattggctagagaagaacaaattgaacaaccaagacaatctggttgtggtgctggtgttactgctggtgttgctattgctaaaactattagattggaatctgaagttactgctattaaaaatgctttgaaaaaaactaatgaagctgtttctactttgggtaatggtgttagagttttggctactgctgttagagaattgaaagattttgtttctaaaaatttgactagagctattaataaaaataaatgtgatattgctgatttgaaaatggctgtttctttttctcaatttaatagaagatttttgaatgttgttagacaattttctgataatgctggtattactccagctatttctttggatttgatgactgatgctgaattggctagagctgtttctaatatgccaacttctgctggtcaaattaaattgatgttggaaaatagagctatggttagaagaaaaggttttggttttttgattggtgtttatggttcttctgttatttatatggttcaattgccaatttttggtgttattgatactccatgttggattgttaaagctgctccatcttgttctgaaaaaaaaggtaattatgcttgtttgttgagagaagatcaaggttggtattgtcaaaatgctggttctactgtttattatccaaatgaaaaagattgtgaaactagaggtgatcatgttttttgtgatacttgtgctggtattaatgttgctgaacaatctaaagaatgtaatattaatatttctactactaattatccatgtaaagtttctactggtagacatccaatttctatggttgctttgtctccattgggtgctttggttgcttgttataaaggtgtttcttgttctattggttctaatagagttggtattattaaacaattgaataaaggttgttcttatattactaatcaagatgctgatactgttactattgataatactgtttatcaattgtctaaagttgaaggtgaacaacatgttattaaaggtagaccagtttcttcttcttttgatccagttaaatttccagaagatcaatttaatgttgctttggatcaagtttttgaatctattgaaaattctcaagctttggttgatcaatctaatagaattttgtctgctggttatattccagaagctccaagagatggtcaagcttatgttagaaaagatggtgaatgggttttgttgtctacttttttgggtggtttggttccaagaggttctcatcatcatcatcatcattctgcttggtctcatccacaatttgaaaaatga

SEQ ID NO: 34 - DNA, 1572 bp, Artificial sequence
  Synthetic
atgtcttggaaagttatgattattatttctttgttgattactccacaacatggtttgaaagaatcttatttggaagaatcttgttctactattactgaaggttatttgtctgttttgagaactggttggtatactaatgtttttactttggaagttggtgatgttgaaaatttgacttgtactgatggtccatctttgattaaaactgaattggatttgactaaatctgctttgagagaattgaaaactgtttctgctgatcaattggctagagaagaacaaattgaacaaccaagacaatctggttgtggtgctggtgttactgctggtattgctattgctaaaactattagattggaatctgaagttaatgctattaaaggtgctttgaaacaaactaatgaagctgtttctactttgggtaatggtgttagagttttggctactgctgttagagaattgaaagaatttgtttctaaaaatttgacttctgctattaatagaaataaatgtgatattgctgatttgaaaatggctgtttctttttctcaatttaatagaagatttttgaatgttgttagacaattttctgataatgctggtattactccagctatttctttggatttgatgactgatgctgaattggctagagctgtttcttatatgccaacttctgctggtcaaattaaattgatgttggaaaatagagctatggttagaagaaaaggttttggtattttgattggtgtttatggttcttctgttatttatatggttcaattgccaatttttggtgttattgatactccatgttggattattaaagctgctccatcttgttctgaaaaaaatggtaattatgcttgtttgttgagagaagatcaaggttggtattgtaaaaatgctggttctactgtttattatccaaatgaaaaagattgtgaaactagaggtgatcatgttttttgtgatacttgtgctggtattaatgttgctgaacaatctagagaatgtaatattaatatttctactactaattatccatgtaaagtttctactggtagacatccaatttctatggttgctttgtctccattgggtgctttggttgcttgttataaaggtgtttcttgttctattggttctaattgggttggtattattaaacaattgccaaaaggttgttcttatattactaatcaagatgctgatactgttactattgataatactgtttatcaattgtctaaagttgaaggtgaacaacatgttattaaaggtagaccagtttcttcttcttttgatccaattaaatttccagaagatcaatttaatgttgctttggatcaagtttttgaatctattgaaaattctcaagctttggttgatcaatctaataaaattttgaatgctggttatattccagaagctccaagagatggtcaagcttatgttagaaaagatggtgaatgggttttgttgtctacttttttgggtggtttggttccaagaggttctcatcatcatcatcatcattctgcttggtctcatccacaatttgaaaaatga

SEQ ID NO: 35 - DNA, 1590 bp, Artificial sequence
  Synthetic
atgtcttggaaggtggtcatcatcttctccctgctgatcacccctcagcacggcctgaaagagtcctacctggaagagagctgctccaccatcaccgagggctacctgtctgtgctgagaaccggctggtacaccaacgtgttcaccctggaagtgggcgacgtggaaaacctgacctgtgctgatggccccagcctgatcaagaccgagctggacctgaccaagtctgccctgagagaactgaggaccgtgtctgccgatcagctggccagagaggaacagatcgagcagcctagacagtccggatgtggtgctggtgctacagctggcgtggccattgccaagaccatccggctggaatctgaagtgaccgccatcaagaacgccctgaaaaagaccaacgaggccgtgtctaccctcggcaatggcgttagagtgctggcctttgctgtgcgcgagctgaaggacttcgtgtccaagaacctgaccagggctctgaacaagaacaagtgtgatatcgccgacctgaagatggccgtgtcctttagccagttcaaccggcggttcctgaacgtcgtgcggcagttctctgataacgccggcatcacccctgccatcagcctggatctgatgaccgatgccgagctggctagagccgtgtccaacatgcctacctctgccggccagatcaagctgatgctggaaaacagagccatggtccgacggaaaggcttcggctttctgatcggcgtgtacggctcctccgtgatctacatggtgcagctgcctatcttcggcgtgatcgacaccccttgctggatcgtgaaggccgctcctagctgctctgagaagaagggcaactacgcctgcctgctgagagaggaccaaggctggtactgtcagaacgccggctccaccgtgtactaccccaacgagaaggactgcgagacaagaggcgaccacgtgttctgcgatacctgcgctggcatcaatgtggccgagcagtccaaagagtgcaacatcaacatctccaccaccaactatccctgcaaggtgtccaccggcaggcaccctatttccatggtggctctgtctccactgggcgccctggtggcttgttataagggcgtgtcctgctccatcggctccaacagagtgggcatcatcaagcagctgaacaagggctgcagctacatcaccaaccaggacgccgataccgtgaccatcgacaataccgtgtatcagctgtccaaggtggaaggcgagcagcacgtgatcaagggcagacctgtgtcctccagcttcgaccccgtgaagttccctgaggatcagttcaacgtggccctggaccaggtgttcgagtccatcgagaactctcaggctctggtggaccagtccaaccggatcctgtcctctgccgagtctgctatcggcggctatatccccgaggctcctagagatggccaggcctatgttcggaaggatggcgaatgggtgctgctgtctaccttcctcggaggcctggtgcctagaggctctcaccaccatcatcaccactccgcttggtcccatccacagttcgagaagtga

SEQ ID NO: 36 - DNA, 1572 bp, Artificial sequence
  Synthetic
atgtcttggaaggtggtcatcatcttctccctgctgatcacccctcagcacggcctgaaagagtcctacctggaagagagctgctccaccatcaccgagggctacctgtctgtgctgagaaccggctggtacaccaacgtgttcatgctggaagtgggcgacgtggaaaacctgacctgtgctgatggccccagcctgctgaaaacagagctggacctgaccaagagcgccctgagaaatctgaggaccgtgtctgccgatcagctggccagagaggaacagatcgagcagcctagacagtccggatgtggtgctggtgctacagctggcgtggccattgccaagaccatccggctggaatctgaagtgaccgccatcaagaatgccctgaaaaagaccaacgaggccgtgtctaccctcggcaatggcgttagagtgctggccacaatggtccgagagctgaaggacttcgtgtccaagaacctgaccagggccatcaacaagaacaagtgtgatatcgccgacctgaagatggccgtgtcctttagccagttcaaccggcggttcctgaacgtcgtgcggcagttctctgataacgccggcatcacccctgccatcagcctggatctgatgaccgatgccgagctggctagagccgtgtccaacatgcctacctctgccggccagatcaagctgatgctcgagaacagagctatggtccgacggaaaggcttcggcatcctgatcggcgtgtacggctcctccgtgatctacatggtgcagctgcctatcttcggcgtgatcgacaccccttgctggatcgtgaaggccgctcctagctgctctgagaagaagggcaactacgcctgcctgctgagagaggaccaaggctggtactgtcagaacgccggctccaccgtgtactaccccaacgagaaggactgcgagacaagaggcgaccacgtgttctgcgatacctgcgctggcatcaatgtggccgagcagtccaaagagtgcaacatcaacatctccaccaccaactatccctgcaaggtgtccaccggcaggcaccctatttccatggtggctctgtctccactgggcgccctggtggcttgttataagggcgtgtcctgctccatcggctccaacagagtgggcatcatcaagcagctgaacaagggctgcagctacatcaccaaccaggacgccgataccgtgaccatcgacaataccgtgtatcagctgtccaaggtggaaggcgagcagcacgtgatcaagggcagacctgtgtcctccagcttcgaccccgtgaagttccctgaggatcagttcaacgtggccctggaccaggtgttcgagtccatcgagaactctcaggctctggtggaccagtccaaccggattctgtctgccggctacatccccgaggctcctagagatggacaggcctacgtcagaaaggacggcgaatgggtgctgctgtctacctttctcggaggcctggtgcctagaggctctcaccaccatcatcaccactccgcttggtcccatcctcagttcgagaagtga

SEQ ID NO: 37 - DNA, 1572 bp, Artificial sequence
  Synthetic
atgtcttggaaggtggtcatcatcttctccctgctgatcacccctcagcacggcctgaaagagtcctacctggaagagagctgctccaccatcaccgagggctacctgtctgtgctgagaaccggctggtacaccaacgtgttcatgctgtgtgtgggcgacgtggaaaacctgacctgtgctgatggccccagcctgctgaaaacagagctggacctgaccaagagcgccctgagagaactgaggaccgtgtctgcagatcagctggccagagaggaacagatcgagcagcctagacagtccggatgtggtgctggtgctacagctggcgtggccattgccaagaccatccggctggaatctgaagtgaccgccatcaagaatgccctgaaaaagaccaacgaggccgtgtctaccctcggcaatggcgttagagtgctggccacaatggtccgagagctgtgcgacttcgtgtccaagaatctgacccgggccatcaacaagaacaagtgtgatatcgccgacctgaagatggccgtgtccttcagccagttcaaccggcggttcctgaatgtcgtgcggcagttctctgacaacgccggcatcacccctgccatcagcctggatctgatgaccgatgccgagctggctagagccgtgtccaacatgcctacctctgccggccagatcaagctgatgctcgagaacagagctatggtccgacggaaaggcttcggcttcctgatcggcgtgtacggctctgacgtgatctacatggtgcagctgcctatcttcggcgtgatcgacaccccttgctggatcgtgaaggccgctcctagctgctctgagaagaagggcaactacgcctgcctgctgagagaggaccaaggctggtactgtcagaacgccggctccaccgtgtactaccccaacgagaaggactgcgagacaagaggcgaccacgtgttctgcgatacctgcgctggcatcaatgtggccgagcagtccaaagagtgcaacatcaacatctccaccaccaactatccctgcaaggtgtccaccggcagacaccccatttccatggtggctctgtctccactgggtgccctggtggcttgttataagggcgtgtcctgctccatcggctccaacagagtgggcatcatcaagcagctgaacaagggctgcagctacatcaccaaccaggacgccgataccgtgaccatcgacaataccgtgtatcagctgtccaaggtggaaggcgagcagcacgtgatcaagggcagacctgtgtcctccagcttcgaccccgtgaagttccctgaggatcagttcaacgtggccctggaccaggtgttcgagtccatcgagaactctcaggctctggtggaccagtccaacagatgctgttccgccggctacatccccgaggctcctagagatggacaggcctacgtcagaaaggacggcgaatgggtgctgctgtctacctttctcggaggcctggtgcctagaggctctcaccaccatcatcaccactccgcttggtcccatccacagttcgagaagtga

SEQ ID NO: 38 - DNA, 1590 bp, Artificial sequence
  Synthetic
atgtcttggaaggtggtcatcatcttctccctgctgatcacccctcagcacggcctgaaagagtcctacctggaagagagctgctccaccatcaccgagggctacctgtctgtgctgagaaccggctggtacaccaacgtgttcatgctggaagtgggcgacgtggaaaacctgacctgtgctgatggccccagcctgatcaagaccgagctggacctgaccaagtctgccctgagagaactgaggaccgtgtctgccgatcagctggccagagaggaacagatcgagcagcctagacagtccggatgtggtgctggtgctacagctggcgtggccattgccaagaccatccggctggaatctgaagtgaccgcctggaagaacgccctgaaaaagaccaacgaggtggtgtctaccctcggcaacggcgtcagagtgctggtcacaatggtccgagagctgaaggacttcgtgtccaagaacctgaccagggctctgaacaagaacaagtgtgatatcgccgacctgaagatggccgtgtctttcagccagttcaaccggcggttcctgaacgtcgtgcggcagttctctgataacgccggcatcacccctgccatcagcctggatctgatgaccgatgccgagctggctagagccgtgtccaacatgcctacctctgccggccagatcaagctgatgctggaaaacagagccatggtccgacggaaaggcttcggctttctgatcggcgtgtacggctcctccgtgatctacatggtgcagctgcctatcttcggcgtgatcgacaccccttgctggatcgtgaaggccgctcctagctgctctgagaagaagggcaactacgcctgcctgctgagagaggaccaaggctggtactgtcagaacgccggctccaccgtgtactaccccaacgagaaggactgcgagacaagaggcgaccacgtgttctgcgatacctgcgctggcatcaatgtggccgagcagtccaaagagtgcaacatcaacatctccaccaccaactatccctgcaaggtgtccaccggcaggcaccctatttccatggtggctctgtctccactgggcgccctggtggcttgttataagggcgtgtcctgctccatcggctccaacagagtgggcatcatcaagcagctgaacaagggctgcagctacatcaccaaccaggacgccgataccgtgaccatcgacaataccgtgtatcagctgtccaaggtggaaggcgagcagcacgtgatcaagggcagacctgtgtcctccagcttcgaccccgtgaagttccctgaggatcagttcaacgtggccctggaccaggtgttcgagtccatcgagaactctcaggctctggtggaccagtccaaccggatcctgtcctctgccgagtctgctatcggcggctatatccccgaggctcctagagatggccaggcctatgttcggaaggatggcgaatgggtgctgctgtctaccttcctcggaggcctggtgcctagaggctctcaccaccatcatcaccactccgcttggtcccatccacagttcgagaagtga

SEQ ID NO: 39 - DNA, 1623 bp, Artificial sequence
  Synthetic
atgtcttggaaggtggtcatcatcttctccctgctgatcacccctcagcacggcctgaaagagtcctacctggaagagagctgctccaccatcaccgagggctacctgtctgtgctgagaaccggctggtacaccaacgtgttcaccctggaagtgggcgacgtggaaaacctgacctgtgctgatggccccagcctgatcaagaccgagctggacctgaccaagtctgccctgagagaactgaggaccgtgtctgccgatcagctggccagagaggaacagatcgagaaccctcggcagtccagattcgtgctgggagctattgctctgggcgtgtgtacagccgctgctgtgacagctggtgtcgctatcgccaagaccatccggctggaatctgaagtgaccgccatcaagaacgccctgaaaaagaccaacgaggccgtgtccacactcggcaatggcgttagagtgctggcctttgctgtgcgcgagctgaaggacttcgtgtccaagaacctgaccagggctctgaacaagaacaagtgtgatatcgccgacctgaagatggccgtgtctttcagccagttcaaccggcggttcctgaacgtcgtgcggcagttctctgataacgccggcatcacccctgccatcagcctggatctgatgaccgatgccgagctggctagagccgtgtctaacatgcctacctctgccggccagatcaagctgatgctggaaaacagagccatggtccgacggaaaggcttcggctttctgatcggcgtgtacggctcctccgtgatctacatggtgcagctgcctatcttcggcgtgatcgacaccccttgctggatcgtgaaggccgctcctagctgctctgagaagaagggcaactacgcctgcctgctgagagaggaccaaggctggtactgtcagaacgccggctccaccgtgtactaccccaacgagaaggactgcgagacaagaggcgaccacgtgttctgcgataccgcctgtggcatcaatgtggccgagcagtccaaagagtgcaacatcaacatctccaccaccaactatccctgcaaggtgtccaccggcaggcaccctatttccatggtggctctgtctccactgggcgccctggtggcttgttataagggcgtgtcctgctccatcggctccaacagagtgggcatcatcaagcagctgaacaagggctgcagctacatcaccaaccaggacgccgataccgtgaccatcgacaataccgtgtatcagctgtccaaggtggaaggcgagcagcacgtgatcaagggcagacctgtgtcctccagcttcgaccccgtgaagttccctgaggatcagttcaacgtggccctggaccaggtgttcgagtccatcgagaactctcaggctctggtggaccagtccaaccggatcctgtcctctgccgagtctgctatcggcggctatatccccgaggctcctagagatggccaggcctatgttcggaaggatggcgaatgggtgctgctgtctaccttcctcggaggcctggtgcctagaggctctcaccaccatcatcaccactccgcttggtcccatccacagttcgagaagtga

SEQ ID NO: 40 - DNA, 1617 bp, Artificial sequence
  Synthetic
atgtcctggaaggtcgtgatcatcttctccctgctgatcaccccccagcacggcctgaaagagtcctacctggaagagagctgctccaccatcaccgagggctacctgtctgtgctgcggaccggctggtacaccaacgtgttcaccctggaagtgggcgacgtggaaaacctgacctgcgccgatggccccagcctgatcaagaccgagctggacctgaccaagtccgccctgcgggaactgagaaccgtgtctgccgatcagctggccagagaggaacagatcgagaacccccggcagtccaagaaacggaagcggagagtggccaccgccgctgctgtgacagctggcgtggccattgccaagaccatccggctggaatccgaagtgaccgccatcaagaacgccctgaaaaagaccaacgaggccgtgtctaccctgggcaatggcgtgcgagtgctggctacagctgtgcgcgagctgaaggacttcgtgtccaagaacctgacccgggccatcaacaagaacaagtgtgatatcgccgacctgaagatggccgtgtcctttagccagttcaaccggcggttcctgaacgtcgtgcggcagttctctgacaacgccggcatcacccctgccatctccctggatctgatgaccgacgccgagctggctagagccgtgtccaacatgcctacctctgccggccagatcaagctgatgctggaaaaccgggccatggtgcgacggaagggcttcggctttctgatcggcgtgtacggctcctccgtgatctacatggtgcagctgcctatcttcggcgtgatcgacaccccctgctggatcgtgaaggccgctcctagctgctccgagaagaagggcaactacgcctgcctgctgagagaggaccagggctggtactgtcagaacgccggctccaccgtgtactaccccaacgagaaggactgcgagacacggggcgaccacgtgttctgtgataccgctgctggcatcaacgtggccgagcagtccaaagagtgcaacatcaacatctccaccaccaactacccctgcaaggtgtccaccggcaggcaccccatctctatggtggccctgtctcctctgggcgccctggtggcttgttacaagggcgtgtcctgctccatcggctccaacagagtgggcatcatcaagcagctgaacaagggctgcagctacatcaccaaccaggacgccgacaccgtgaccatcgacaataccgtgtatcagctgtccaaggtggaaggcgagcagcacgtgatcaagggcagacccgtgtcctccagcttcgaccccgtgaagttccccgaggatcagttcaatgtggccctggaccaggtgttcgagtccatcgagaactcccaggctctggtggaccagtccaaccggatcctgtcctctgccgagaagggaaacacctccggcagagagaacctgtattttcaaggcggcggaggctccggctacatccctgaggctcctagagatggccaggcctacgtgcggaaggatggcgaatgggtgctgctgtccaccttcctgggcggcatcgagggcagacaccaccatcatcaccactga

SEQ ID NO: 41 - protein, 276 aa, Human metapneumovirus
MGHHHHHHHHHHSSGHIDDDDKQESYLVDTYQGIPYTAAVQVDLVEKDLLPASLTIWFPLFQANTPPAVLLDQLKTLTITTLYAASQSGPILKVNASAQGAAMSVLPKKFEVNATVALDEYSKLEFDKLTVCEVKTVYLTTMKPYGMVSKFVSSAKPVGKKTHDLIALCDFMDLEKNTPVTIPAFIKSVSIKESESATVEAAISSEADQALTQAKIAPYAGLIMIMTMNNPKGIFKKLGAGTQVIVELGAYVQAESISKICKTWSHQGTRYVLKSR

SEQ ID NO: 42 - protein, 18 aa, Artificial sequence
  Synthetic
CCKQTNECCKNLERAVSA

SEQ ID NO: 43 - protein, 18 aa, Artificial sequence
  Synthetic
CCRELKECCKNLENAVSA

SEQ ID NO: 44 - protein, 18 aa, Artificial sequence
  Synthetic
CCRELKDCCKNLENAVSA

SEQ ID NO: 45 - protein, 18 aa, Artificial sequence
  Synthetic
CCRELKDCCKNLERAVSA

SEQ ID NO: 46 - protein, 18 aa, Artificial sequence
  Synthetic
CCRELKDCCKQLNKAVSA

SEQ ID NO: 47 - protein, 18 aa, Artificial sequence
  Synthetic
CCRELKECCKQLNKAVSA

SEQ ID NO: 48 - DNA, 1566 bp, Artificial sequence
  Synthetic
atgatcattatctccctgctgatcaccccccagcacggcctgaaagagtcctacctggaagagagctgctccaccatcaccgagggctacctgtctgtgctgcggaccggctggtacaccaacgtgttcaccctggaagtgggcgacgtggaaaacctgacctgcaccgatggccccagcctgatcaagaccgagctggacctgaccaagtccgccctgcgcgagctgaaaaccgtgtctgccgatcagctggccagagaggaacagatcgagaacccccggcagtccaagaaacggaagcggagagtggccaccgccgctgctgtgacagctggaatcgctatcgccaagaccatccggctggaatccgaagtgaacgccatcaagggcgctctgaagcagaccaacgaggccgtgtctaccctgggcaatggcgtgcgagtgctggctacagctgtgcgggaactgaaagaattcgtgtccaagaacctgaccagcgccatcaaccggaacaagtgtgatatcgccgacctgaagatggccgtgtccttcagccagttcaaccggcggttcctgaacgtcgtgcggcagttctctgacaacgccggcatcacccctgccatctccctggatctgatgaccgacgccgagctggctagagccgtgtcttacatgcctacctctgccggccagatcaagctgatgctggaaaaccgggccatggtgcgacggaagggcttcggcatcctgatcggcgtgtacggctcctccgtgatctacatggtgcagctgcctatcttcggcgtgatcgacaccccctgctggattatcaaggccgctcccagctgctccgagaagaacggcaactacgcctgcctgctgagagaggaccagggctggtactgcaagaacgccggctccaccgtgtactaccccaacgagaaggactgcgagacacggggcgaccacgtgttctgtgataccgctgctggcatcaacgtggccgagcagtccagagagtgcaacatcaacatctccaccaccaactacccctgcaaggtgtccaccggcaggcaccccatctctatggtggccctgtctcctctgggagccctggtggcttgttacaagggcgtgtcctgctccatcggctccaactgggtgggaatcatcaagcagctgcccaagggctgcagctacatcaccaaccaggacgccgacaccgtgaccatcgacaataccgtgtatcagctgtccaaggtggaaggcgagcagcacgtgatcaagggcagacccgtgtccagctccttcgaccccatcaagttccccgaggatcagttcaatgtggccctggaccaggtgttcgagtccatcgagaactcccaggctctggtggaccagtccaacaagatcctgaactccgccgagaagggcaacacctccggcagagagaacctgtattttcaaggcggcggaggctccggctacatccctgaggctcctagagatggccaggcctacgtgcggaaggatggcgaatgggtgctgctgtccaccttcctgtga

SEQ ID NO: 49 - protein, 539 aa, Human metapneumovirus
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNSAEKGNTGFIIVVILVAVLGLTMISVSIIIIIKKTRKPTGAPPELNGVTNGGFIPHS

SEQ ID NO: 50 - protein, 529 aa, Artificial sequence
  Synthetic
MSWKVMIIISLLITPQHGLKESYLEESCSTITEGYLSVLRTGWYTNVFTLEVGDVENLTCTDGPSLIKTELDLTKSALRELKTVSADQLAREEQIEQPRQSGCGAGATAGIAIAKTIRLESEVNAIKGALKQTNEAVSTLGNGVRVLAFAVRELKEFVSKNLTSALNRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTCAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNSAESAIGGYIPEAPRDGQAYVRKDGEWVLLSTFLGGLVPRGSHHHHHHSAWSHPQFEK

SEQ ID NO: 51 - protein, 523 aa, Artificial sequence
  Synthetic
MSWKVMIIISLLITPQHGLKESYLEESCSTITEGYLSVLRTGWYTNVFMLEVGDVENLTCTDGPSLLKTELDLTKSALRNLKTVSADQLAREEQIEQPRQSGCGAGATAGIAIAKTIRLESEVNAIKGALKQTNEAVSTLGNGVRVLATMVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTCAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNAGYIPEAPRDGQAYVRKDGEWVLLSTFLGGLVPRGSHHHHHHSAWSHPQFEK

SEQ ID NO: 52 - protein, 523 aa, Artificial sequence
  Synthetic
MSWKVMIIISLLITPQHGLKESYLEESCSTITEGYLSVLRTGWYTNVFMLCVGDVENLTCTDGPSLLKTELDLTKSALRELKTVSADQLAREEQIEQPRQSGCGAGATAGIAIAKTIRLESEVNAIKGALKQTNEAVSTLGNGVRVLATMVRELCEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSDVIYMVQLPIFGVIDTPCWIIKAAPSCSEKNGNYACLLREDQGWYCKNAGSTVYYPNEKDCETRGDHVFCDTCAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKCCNAGYIPEAPRDGQAYVRKDGEWVLLSTFLGGLVPRGSHHHHHHSAWSHPQFEK

SEQ ID NO: 53 - protein, 529 aa, Artificial sequence
  Synthetic
MSWKVMIIISLLITPQHGLKESYLEESCSTITEGYLSVLRTGWYTNVFMLEVGDVENLTCTDGPSLIKTELDLTKSALRELKTVSADQLAREEQIEQPRQSGCGAGATAGIAIAKTIRLESEVNAWKGALKQTNEVVSTLGNGVRVLVTMVRELKEFVSKNLTSALNRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTCAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNSAESAIGGYIPEAPRDGQAYVRKDGEWVLLSTFLGGLVPRGSHHHHHHSAWSHPQFEK